(19) United States
(12) Patent Application Publication
(10) Pub. No.: US 2026/0130848 A1
(43) Pub. Date: May 14, 2026

(54) METHODS AND COMPOSITIONS FOR CENTRAL NERVOUS SYSTEM (CNS) DRUG DELIVERY AND TREATMENT

(71) Applicant: University of Massachusetts, Westborough, MA (US)

(72) Inventors: Rachael Sirianni, Princeton, MA (US); Nils Henninger, Shrewsbury, MA (US)

(21) Appl. No.: 19/383,567

(22) Filed: Nov. 7, 2025

Related U.S. Application Data
(60) Provisional application No. 63/718,560, filed on Nov. 8, 2024.

(51) Int. Cl. A61K 9/00; A61K 9/08; A61K 45/00; A61K 47/02; A61P 25/00; A61P 25/28

(52) U.S. Cl. CPC A61K 9/0085; A61K 9/0019; A61K 9/08; A61K 45/00; A61K 47/02; A61P 25/00; A61P 25/28

(57) ABSTRACT

The disclosure herein provides, in example embodiments, methods and compositions for central nervous system (CNS) drug delivery and treatment comprising administering an infusion solution to a cerebrospinal fluid (CSF) of the subject, wherein the infusion solution comprises a hypertonic solution. The disclosure also provides compositions comprising a hypertonic solution for use in treatment and drug delivery in the CNS.

### RELATED APPLICATION

This application claims the benefit of U.S. Provisional Application No. 63/718,560, filed on Nov. 8, 2024. The entire teachings of the above application are incorporated herein by reference.

### GOVERNMENT SUPPORT

This invention was made with government support under HD099543, and NS 111292N, and NS 116657 from National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith: File name: 5439.1038-001SL.xml; created Nov. 7, 2025, 4, 377 Bytes in size.

### BACKGROUND

Diseases and injuries affecting the central nervous system (CNS), such as neurodegenerative diseases, leptomeningeal metastasis (LM), and traumatic brain injury (TBI) are challenging to treat. LM is a prevalent complication of multiple types of cancer involving the pia and arachnoid mater of the brain with the subarachnoid space in between. Because LM cannot be surgically resected and is often unresponsive to systemic chemotherapy, patients are generally treated with high dose chemoradiotherapy (CRT), which yields severe treatment-associated sequelae and very poor long-term outcomes. Recurrent LM is untreatable and drug delivery remains a primary obstacle to the treatment of LM. TBI is a major health burden and an important risk factor for several progressive neurodegenerative disorders, including Parkinson's disease (PD), amyotrophic lateral sclerosis (ALS), Alzheimer's disease (AD), and Alzheimer's disease-related dementias (ADRDs) such as frontotemporal dementia (FTD). Accordingly, there is a need for improved methods and compositions for treating diseases and injuries affecting the central nervous system (CNS), including, for example, LM, TBI, PD, ALS, AD, ADRDs, and FTD.

### SUMMARY

There is a critical need to develop improved methods for treating diseases and injuries affecting the central nervous system (CNS). The disclosure provides such methods and treatments.

In one aspect, the disclosure herein provides a method for enhancing delivery of a therapeutic agent to a parenchyma or a perivascular space of a subject in need thereof, said method comprising administering an infusion solution to a cerebrospinal fluid (CSF) of the subject, wherein the infusion solution comprises a hypertonic solution and the therapeutic agent.

In another aspect, the disclosure herein provides a method for treating a subject in need thereof, said method comprising administering an infusion solution to a cerebrospinal fluid (CSF) of the subject, wherein the infusion solution comprises a hypertonic solution.

In another aspect, the disclosure herein provides a method for enhancing cerebrospinal fluid distribution in a subject in need thereof, said method comprising administering an infusion solution to a cerebrospinal fluid (CSF) of the subject, wherein the infusion solution comprises a hypertonic solution.

In another aspect, the disclosure herein provides a method for reducing an abnormal protein deposit in a subject in need thereof, said method comprising administering an infusion solution to a cerebrospinal fluid (CSF) of the subject, wherein the infusion solution comprises a hypertonic solution.

In another aspect, the disclosure herein provides a method for stimulating a choroid plexus (CP) to produce cerebrospinal fluid (CSF) in a subject in need thereof, said method comprising administering an infusion solution to the CSF of the subject, wherein the infusion solution comprises a hypertonic solution.

In another aspect, the disclosure herein provides a method for stimulating production of a cerebrospinal fluid (CSF) in a subject in need thereof, said method comprising administering an infusion solution to the CSF of the subject, wherein the infusion solution comprises a hypertonic solution.

In another aspect, the disclosure herein provides a method for treating a disease or injury of the central nervous system (CNS) in subject in need thereof, said method comprising administering an infusion solution to a cerebrospinal fluid (CSF) of the subject, wherein the infusion solution comprises a hypertonic solution.

In some embodiments, the hypertonic solution has an osmolarity at least about 1.1 times (1.1×) an osmolarity of the CSF of the subject.

In some embodiments, the hypertonic solution has an osmolarity of up to about 8 times (8×) an osmolarity of the CSF of the subject.

In some embodiments, the hypertonic solution comprises one or more electrolytes selected from sodium (Na⁺), potassium (K⁺), Calcium (Ca²⁺), magnesium (Mg²⁺), chloride (Cl⁻), and biocarbonate (HCO₃⁻), wherein an osmolarity of each of the one or more electrolytes is higher than an osmolarity of said electrolytes in the CSF of the subject.

In some embodiments, the subject is a rat and the hypertonic solution has an osmolarity of:
a) up to about 2640 milliosmoles (mOsm),
b) about 363 mOsm to about 1320 mOsm, or
c) about 495 mOsm to about 660 mOsm.

In some embodiments, the subject is a human and the hypertonic solution has an osmolarity of:
a) up to about 2240 mOsm,
b) about 308 mOsm to about 1120 mOsm, or
c) about 420 mOsm to about 560 mOsm.

In some embodiments, the subject is a mouse and the hypertonic solution has an osmolarity of:
a) up to about 2320 mOsm,
b) about 319 mOsm to about 1160 mOsm, or
c) about 435 mOsm to about 580 mOsm.

In some embodiments, the hypertonic solution comprises:
a) about 100 to about 200 mOsm Na⁺,
b) about 2 to about 4 mOsm K⁺,
c) about 1 to about 2.5 mOsm Ca²⁺,
d) about 1 to about 3 mOsm Mg²⁺,
e) about 100 to about 200 mOsm Cl⁻,
f) about 18 to about 27 mOsm HCO₃⁻, or
g) any combination of the foregoing.

In some embodiments, the hypertonic solution comprises:
a) about 125 mOsm Na⁺,
b) about 3 mOsm K⁺,
c) about 2.3 mOsm Ca²⁺,
d) about 2.2 mOsm Mg²⁺,
e) about 134 mOsm Cl⁻,
f) about 23 mOsm HCO₃⁻, or
g) any combination of the foregoing.

In some embodiments, the hypertonic solution comprises:
a) about 137 to about 1600 mOsm Na⁺,
b) about 3 to about 25 mOsm K⁺,
c) about 1 to about 16 mOsm Ca²⁺,
d) about 1 to about 9 mOsm Mg²⁺,
e) about 124 to about 1072 mOsm Cl⁻,
f) about 25 to about 208 mOsm HCO₃⁻, or
g) any combination of the foregoing.

In some embodiments, the hypertonic solution comprises:
a) a total osmolarity of about 1.1 times (1.1×) to about 8 times (8×) a total osmolarity of the CSF of the subject,
b) an osmolarity of Na⁺ about 1.1 times (1.1×) to about 8 times (8×) an osmolarity of Na⁺ in the CSF of the subject,
c) an osmolarity of K⁺ about 1.1 times (1.1×) to about 8 times (8×) an osmolarity of K⁺ in the CSF of the subject,
d) an osmolarity of Ca²⁺ about 1.1 times (1.1×) to about 8 times (8×) an osmolarity of Ca²⁺ in the CSF of the subject,
e) an osmolarity of Mg²⁺ about 1.1 times (1.1×) to about 8 times (8×) an osmolarity of Mg²⁺ in the CSF of the subject,
f) an osmolarity of Cl⁻ about 1.1 times (1.1×) to about 8 times (8×) an osmolarity of Cl⁻ in the CSF of the subject,
g) an osmolarity of HCO₃⁻ about 1.1 times (1.1×) to about 8 times (8×) an osmolarity of HCO₃ in the CSF of the subject, or h) any combination of the foregoing.

In some embodiments, the hypertonic solution has an osmolarity of about 308 to about 2480.

In some embodiments, the hypertonic solution has a pH of about 6 to about 9.

In some embodiments, the hypertonic solution comprises a hypertonic artificial CSF (aCSF) or a hypertonic phosphate buffered solution (PBS).

In some embodiments, the infusion solution further comprises a therapeutic agent.

In some embodiments, the method further comprises the step of administering a therapeutic agent to the subject before, during, or after administering the infusion solution to the subject.

In some embodiments, the therapeutic agent comprises a nanoparticle, DNA, RNA, protein, peptide, lipid, liposome, viral vector, small molecule, large molecule, aptamer, chemotherapeutic, or a combination thereof.

In some embodiments, the DNA comprises a genomic DNA (gDNA), complementary DNA (cDNA), oligonucleotide, antisense oligonucleotide (ASO), or a combination thereof.

In some embodiments, the RNA comprises an antisense RNA (asRNA), short interfering RNA (siRNA), oligonucleotide, antisense oligonucleotide (ASO), or a combination thereof.

In some embodiments, the protein comprises an antibody.

In some embodiments, the antibody comprises an immunoglobulin G (IgG).

In some embodiments, the viral vector is an adeno-associated virus (AAV) vector.

In some embodiments, the nanoparticle comprises polystyrene, polyester, a nucleic acid, or poly(lactic-co-glycolic) acid (PLGA).

In some embodiments, the nanoparticle comprises a 20 nm polystyrene sphere, a 40 nm polystyrene sphere, a 100 nm polystyrene sphere, or a polyester sphere.

In some embodiments, the nanoparticle or a portion thereof is spherical, cylindrical, conical, torpedo-shaped, deformable, or a combination thereof.

In some embodiments, an aspect ratio of the nanoparticle is about 1:1 to 1:50.

In some embodiments, a dimension of the therapeutic agent is up to about 1 micron, for example, about 500 nm.

In some embodiments, a dimension of the therapeutic agent is about 100 to 400 nm.

In some embodiments, a dimension of the therapeutic agent is about 200 to 300 nm.

In some embodiments, a dimension of the therapeutic agent is up to about 100 nm.

In some embodiments, the therapeutic agent is charged.

In some embodiments, the therapeutic agent is lipophilic or hydrophilic.

In some embodiments, the infusion solution is administered intrathecally, for example:
at a lateral ventricle, third ventricle, fourth ventricle, cisterna magna, or lumbar subarachnoid space of the subject;
proximally to a choroid plexus of the subject;
or a combination of any of the foregoing.

In some embodiments, the infusion solution is administered at a rate of up to about 20 L over up to about 20-80 seconds.

In some embodiments, the infusion solution is administered at a rate of about 10 μL over about 28-33 seconds.

In some embodiments, the administering the infusion solution is performed with a 30 gauge needle or a 33 gauge needle.

In some embodiments, the infusion solution has a volume of up to about half of a volume of a CSF of the subject.

In some embodiments, the infusion solution has a volume of about one-fifth to about one half of a volume of a CSF of the subject.

In some embodiments, the subject has, is suspected to have, or is at risk for having a stroke, neurodegenerative disease, neuroinflammation, cancer affecting a central nervous system (CNS), disease or infection of the CNS, cerebrovascular disease, or a combination thereof.

In some embodiments, the subject has or is suspected to have a traumatic brain injury (TBI), traumatic spinal cord injury, or both.

In some embodiments, the TBI comprises a recurrent TBI (rTBI), single TBI (sTBI), or both.

In some embodiments, the infusion solution is administered within about 48 hours, within about 24 hours, within about 8 hours, or within about 4 hours of the traumatic brain injury (TBI) or traumatic spinal cord injury. In some embodiments, the infusion solution is administered within about 24 hours of the traumatic brain injury (TBI) or traumatic spinal cord injury. In some embodiments, the infusion solution is administered within about 8 hours of the traumatic brain injury (TBI) or traumatic spinal cord injury. In some embodiments, the infusion solution is administered within about 4 hours of the traumatic brain injury (TBI) or traumatic spinal cord injury.

In some embodiments, the neurodegenerative disease comprises or is associated with amyotrophic lateral sclerosis (ALS), frontotemporal dementia (FTD), multiple sclerosis (MS), Alzheimer's disease and related dementias (ADRD), Guillain-Barré syndrome, hexanucleotide repeat expansion (HRE) in a C9orf72 gene, C9orf72 haploinsufficiency, cytoplasmic accumulation of TAR DNA-binding protein 43 kDa (TDP-43), or a combination thereof.

In some embodiments, the cancer is primary, metastatic, associated with leptomeningeal metastases (LM), or a combination thereof.

In some embodiments, the cancer comprises brain cancer, a brain metastasis, or both. In some embodiments, the cancer comprises brain cancer. In some embodiments, the cancer comprises a brain metastasis.

In some embodiments, the cancer comprises medulloblastoma (MB).

In some embodiments, the disease of the CNS is a lysosomal storage disorder.

In some embodiments, the subject has, is suspected to have, or is at risk for having an abnormal deposit comprising a neurotoxin, an inflammatory cytokine, or both.

In some embodiments, the neurotoxin is a small molecule or a protein.

In some embodiments, the abnormal deposit comprises amyloid-β, Tau protein, α-synuclein, or a combination thereof.

In some embodiments, the subject is a pediatric subject.

In some embodiments, the subject is an adult subject.

In another aspect, the disclosure herein provides an infusion solution for use in enhancing delivery of a therapeutic agent to a parenchyma or a perivascular space of a subject in need thereof, comprising a hypertonic solution and a therapeutic agent.

In another aspect, the disclosure herein provides an infusion solution for use in treating a subject in need thereof, comprising a hypertonic solution.

In another aspect, the disclosure herein provides an infusion solution for use in enhancing cerebrospinal fluid flow in a subject in need thereof, comprising a hypertonic solution.

In another aspect, the disclosure herein provides an infusion solution for use in reducing an abnormal protein deposit in a subject in need thereof, comprising a hypertonic solution.

In another aspect, the disclosure herein provides an infusion solution for use in stimulating production of cerebrospinal fluid (CSF) in a subject in need thereof, comprising a hypertonic solution.

In some embodiments, the infusion solution further comprises a therapeutic agent.

In another aspect, the disclosure herein provides a composition for use in manufacture of a medicament for enhancing delivery of a therapeutic agent to a parenchyma or a perivascular space of a subject in need thereof, comprising an infusion solution, wherein the infusion solution comprises a hypertonic solution and a therapeutic agent.

In another aspect, the disclosure herein provides a composition for use in manufacture of a medicament for treating a subject in need thereof, comprising an infusion solution, wherein the infusion solution comprises a hypertonic solution and a therapeutic agent.

In another aspect, the disclosure herein provides a composition for use in manufacture of a medicament for enhancing cerebrospinal fluid flow in a subject in need thereof, comprising an infusion solution, wherein the infusion solution comprises a hypertonic solution and a therapeutic agent.

In another aspect, the disclosure herein provides a composition for use in manufacture of a medicament for reducing an abnormal protein deposit in a subject in need thereof, comprising an infusion solution, wherein the infusion solution comprises a hypertonic solution and a therapeutic agent.

In another aspect, the disclosure herein provides a composition for use in manufacture of a medicament for stimulating production of cerebrospinal fluid (CSF) in a subject in need thereof, comprising an infusion solution, wherein the infusion solution comprises a hypertonic solution and a therapeutic agent.

In some embodiments, the composition further comprises at least two therapeutic agents.

In some embodiments, the hypertonic solution comprises:
a) a total osmolarity of about 1.1 times (1.1×) to about 8 times (8×) a total osmolarity of the CSF of the subject,
b) an osmolarity of Na⁺ about 1.1 times (1.1×) to about 8 times (8×) an osmolarity of Na⁺ in the CSF of the subject,
c) an osmolarity of K⁺ about 1.1 times (1.1×) to about 8 times (8×) an osmolarity of K⁺ in the CSF of the subject,
d) an osmolarity of Ca²⁺ about 1.1 times (1.1×) to about 8 times (8×) an osmolarity of Ca²⁺ in the CSF of the subject,
e) an osmolarity of Mg²⁺ about 1.1 times (1.1×) to about 8 times (8×) an osmolarity of Mg²⁺ in the CSF of the subject,
f) an osmolarity of Cl⁻ about 1.1 times (1.1×) to about 8 times (8×) an osmolarity of Cl⁻ in the CSF of the subject,
g) an osmolarity of HCO₃ about 1.1 times (1.1×) to about 8 times (8×) an osmolarity of HCO₃ in the CSF of the subject, or
h) any combination of the foregoing.

In another aspect, the disclosure herein provides a kit comprising an infusion solution or a composition and a composition comprising an additional therapeutic agent and/or a device for administration of the solution and/or composition.

In another aspect, the disclosure herein provides a kit comprising a lyophilized form of an infusion solution or a composition and a composition comprising an additional therapeutic agent and/or a device for administration of the solution and/or composition.

In another aspect, the disclosure herein provides a kit comprising a hypertonic solution, wherein the hypertonic solution comprises:
a) a total osmolarity of about 1.1 times (1.1×) to about 8 times (8×) a total osmolarity of the CSF of the subject,
b) an osmolarity of Na⁺ about 1.1 times (1.1×) to about 8 times (8×) an osmolarity of Na⁺ in the CSF of the subject,
c) an osmolarity of K⁺ about 1.1 times (1.1×) to about 8 times (8×) an osmolarity of K⁺ in the CSF of the subject,
d) an osmolarity of Ca²⁺ about 1.1 times (1.1×) to about 8 times (8×) an osmolarity of Ca²⁺ in the CSF of the subject,
e) an osmolarity of Mg²⁺ about 1.1 times (1.1×) to about 8 times (8×) an osmolarity of Mg²⁺ in the CSF of the subject,
f) an osmolarity of Cl⁻ about 1.1 times (1.1×) to about 8 times (8×) an osmolarity of Cl⁻ in the CSF of the subject,
g) an osmolarity of HCO₃ about 1.1 times (1.1×) to about 8 times (8×) an osmolarity of HCO₃ in the CSF of the subject, or
h) any combination of the foregoing.

In another aspect, the disclosure herein provides a kit comprising a lyophilized hypertonic solution, wherein the hypertonic solution comprises:
a) a total osmolarity of about 1.1 times (1.1×) to about 8 times (8×) a total osmolarity of the CSF of the subject,
b) an osmolarity of Na⁺ about 1.1 times (1.1×) to about 8 times (8×) an osmolarity of Na⁺ in the CSF of the subject,
c) an osmolarity of K⁺ about 1.1 times (1.1×) to about 8 times (8×) an osmolarity of K⁺ in the CSF of the subject,
d) an osmolarity of Ca²⁺ about 1.1 times (1.1×) to about 8 times (8×) an osmolarity of Ca²⁺ in the CSF of the subject,
e) an osmolarity of Mg²⁺ about 1.1 times (1.1×) to about 8 times (8×) an osmolarity of Mg²⁺ in the CSF of the subject,
f) an osmolarity of Cl⁻ about 1.1 times (1.1×) to about 8 times (8×) an osmolarity of Cl⁻ in the CSF of the subject,
g) an osmolarity of HCO₃ about 1.1 times (1.1×) to about 8 times (8×) an osmolarity of HCO₃ in the CSF of the subject, or
   any combination of the foregoing.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D. Cerebrospinal fluid distribution. FIG. 1A is a representative pseudocolored positron emission tomography (PET) image of sagittal slice showing ¹⁸F distribution in the brain and upper spinal cord following co-infusion of 20 μL isotonic solution with ¹⁸F. FIG. 1B is an example illustration of how data in subsequent figures are presented according to the anterior-posterior orientation of model animal brains. The x-axis corresponds to positions spanning from the cerebellum/brainstem (lower x-values) to the olfactory bulb (higher x-values); x=0 is the center of infusion in the lateral ventricle. FIGS. 1C-1D show results from administration of isotonic 1× phosphate-buffered saline (PBS), or 5× hypertonic PBS (5×osmolarity) into a lateral ventricle of Sprague Dawley rats by intracerebroventricular (ICV) injection. A separate control group had isotonic 1×PBS infused into the parenchyma by convection-enhanced delivery (CED). All infusions contained F-18 fluoride ion (¹⁸F, radiofluorine), a small, water-soluble molecule used to track fluid movement. Isotonic 1×PBS/ICV, and isotonic 1×PBS/CED are negative controls. FIG. 1C is a series of graphs quantifying anterior-posterior ¹⁸F signal following infusion with a Control (1×PBS/CED), Isotonic (1×PBS/ICV), or Hypertonic (5×PBS/ICV) solution. The anterior-posterior position of the olfactory bulb is marked with an asterisk (*). FIG. 1D is a series of graphs that displays the data from FIG. 1C by timepoint (end of infusion, 1 hour, 3 hours).

FIGS. 2A-2G show results from an experiment designed to assess delivery and efficacy of β-cyclodextrin-poly (β-amino ester) network (CDN₅) nanoparticles (NPs) loaded with the histone deacetylase inhibitor (HDACi) panobinostat (pCDN₅) following intrathecal administration in healthy mice (FIG. 2B) or in mice bearing patient-derived medulloblastoma (MB) exhibiting leptomeningeal metastasis (LM) (FIGS. 2C-2G). FIG. 2A is an illustration of the structure of a CDN₅ nanoparticle comprising acrylated (blue) β-cyclodextrin (red) that can be ⁶⁴Cu-radiolabeled. ⁶⁴Cu-CDN₅ was utilized in an experiment for which results are shown in FIG. 2G. FIG. 2B is a graph quantifying distribution of β-cyclodextrin-solubilized panobinostat (pCD) or pCDN₅ in the spinal cord of C57 mice following intrathecal (IT) administration via the cisterna magna. FIG. 2C is a graph quantifying activation of a therapeutic target for HDACi activity, forkhead box protein O1 (FOXO1) in the cerebellum of mice treated with drug-empty CDN₅ controls (bCDN₅) or pCDN₅. AU, arbitrary units. FIG. 2D is a survival curve showing that pCDN₅ prolonged survival in mice with MB compared to bCDN₅. FIG. 2E is a bar graph quantifying the incidence of LM in mice with MB treated with bCDN₅ or pCDN₅. FIG. 2F is a pair of fluorescent microscopy images showing that fluorescently labeled polystyrene NPs achieved inconsistent access to LM; the two images show the same location in two different mice to which NPs were administered. FIG. 2G is a distribution graph and schematic showing PET signal of ⁶⁴Cu-radiolabeled bCDN₅ NPs in the spine following lumbar or cisterna magna injection. IT-L, intrathecal, lumbar administration site; IT-CM, intrathecal, cisterna magna administration site.

FIG. 3 is an image of a mouse brain (left) indicating the regions of interest in which nanoparticle distribution was examined with whole-field microscopy. Insets (right) that show distribution of gold NPs with different geometries: spherical and 100 nm in diameter (S100), spherical and 10 nm (S10) in diameter, or rod-shaped and 15 nm by 40 nm in size (R15×40).

FIGS. 4A-4B. In vitro cell uptake data. 1, 2, and 3 refer to three examples of stretched (non-spherical) polystyrene nanoparticles.

FIGS. 5A-5C show results from infusion of isotonic (1×) and hypertonic (up to 8×) aCSF containing 100 nm fluorescently (red) labeled polystyrene NPs into the cisterna magna of C57 mice. FIGS. 5A and 5B utilized a 30 G infusion needle. FIG. 5C utilized a 33 G infusion needle. FIG. 5A is a pair of fluorescence images of sagittal slices demonstrating 100 nm NP distribution 1 hour after co-infusion with isotonic or 2×hypertonic aCSF. FIG. 5B is a series of fluorescence images of transverse (axial) slices demonstrating NP distribution 1 hour after co-infusion with isotonic (1×) or hypertonic (2× or 8×) aCSF. FIG. 5C is a series of fluorescence images of coronal slices demonstrating NP distribution 1 hour after co-infusion with isotonic or 2× hypertonic aCSF.

FIGS. 6A-6I show results from infusion of 2× hypertonic aCSF and nucleic acid nanoparticles (NANPs) into the cisterna magna of C57 mice. FIGS. 6A-6D show results from experiments using base NANPs. FIG. 6A is a series of graphs showing the fluorescent signal (“radiant efficiency”) measured by whole-mouse body imaging following infusion of base NANPs in 1× aCSF (M2) or 2×aCSF (M5 (CSF flush)). The graphs quantify signal (775 nm excitation, 800 nm emission) from about 50 nm fluorescently labeled nucleic acid NPs in head and the lower spinal cord (SC) within whole-body fluorescence images. Isotonic and 2×hypertonic artificial CSF (aCSF) containing the NPs was infused into the cisterna magna of C57 mice, yielding enhanced movement of NPs from the brain region of interest (ROI) to the lower spinal cord ROI. One-Way analysis of variance (ANOVA) followed by Tukey post hoc comparison. MDA-MB-231 cells. FIG. 6B contains example stereoscopic images of the ventral surface of the mouse brain showing differences in NANP distribution following infusion of base NANPs in buffer or 2×aCSF. FIG. 6C contains example stereoscopic images of the spinal cord showing differences in NANP distribution following infusion of base NANPs in 1× or 2× aCSF.

FIG. 6D is a whole-body fluorescence image showing NANP distribution in both the brain and spinal cord following infusion of base NANPs in 1× aCSF. FIGS. 6E-6F show results from experiments using PEGylated NANPs, i.e., NANPs whose surface chemistry has been modified with poly(ethylene glycol) (PEG). FIG. 6E contains an example stereoscopic image of the ventral surface of the mouse brain following infusion of PEGylated NANPs in 1× aCSF. FIG. 6F is a whole-body fluorescence image showing signal distribution in the brain and a lack of NANP distribution in the spinal cord following infusion of PEGylated NANPs. FIGS. 6G-6I show results from experiments using lipidated NANPs, i.e., NANPs whose surface chemistry has been modified with lipid moieties. FIG. 6G is a series of graphs showing the fluorescent signal (“radiant efficiency”) measured by whole-mouse body imaging following infusion of lipidated NANPs in 1× aCSF or 2× aCSF. FIG. 6H contains example stereoscopic images of the ventral surface of the mouse brain showing differences in NANP distribution following infusion of lipidated NANPs in 1× or 2× aCSF. FIG. 6I is a whole-body fluorescence image showing signal distribution in the brain and lower spinal cord following infusion of lipidated NANPs.

FIG. 7 is a series of graphs showing the fold-change in relative fluorescence following infusion of the model small molecule rhodamine B in isotonic aCSF and hypertonic 2×aCSF into the cisterna magna of C57 mice.

FIG. 8 is a series of fluorescence images showing the distribution of the small, water-soluble molecule hydrazide or 20-100 nM polystyrene nanoparticles (PSNPs) in brains and spinal cords of mice bearing MDA-MB-231 tumors after co-infusion with isotonic (1×) or hypertonic (2×) aCSF.

FIG. 9 is a series of fluorescence images showing the distribution of polyester nanoparticles (NPs) in spinal cords including bone (bone in; includes tissue, bone, and subarachnoid space) or excluding bone (bone out; tissue only) in mice bearing MDA-MB-231 tumors after co-infusion with isotonic (1×) or hypertonic (2×) aCSF.

FIGS. 10A-10E are a series of graphs quantifying the localization of hydrazide 694 (FIG. 10A), rhodamine (FIG. 10B), and 20 nm (FIG. 10C), 40 nm (FIG. 10D), and 100 nM (FIG. 10E) FLUOSPHERES® carboxylate-modified microspheres in the brain, upper spinal cord, or lower spinal cord of healthy mice after co-infusion with isotonic (1×) or hypertonic (2×) aCSF.

FIGS. 11-12 show distribution of FLUOSPHERES® in the brain, USC, and LSC of healthy mice after co-infusion with isotonic (1×) or hypertonic (2×) aCSF.

FIG. 11 is a series of graphs quantifying the localization of 20 nm, 40 nm, and 100 nM FLUOSPHERES® (FS).

FIG. 12 is a series of graphs quantifying the distribution shown in FIG. 11. Asterisk denotes 40 nm thoracic p=0.03.

FIG. 13 is a series of fluorescence images showing the distribution of 40 nm and 100 nm FLUOSPHERES® (FS) on the lateral surfaces of the mouse brain after co-infusion with isotonic (1×) or hypertonic (2×) aCSF. Arrows indicate regions where meningeal vessels have entered the parenchyma of the brain. Nanoparticles are observed to localize with perivascular spaces.

FIG. 14 is a series of fluorescence images showing the distribution of hydrazide and 100 nm FLUOSPHERES® (FS) in the brain after co-infusion with isotonic (1×) or hypertonic (2×) aCSF. Circled regions of interest indicate cerebrospinal fluid flow enhancement (CFE) of delivery to the hypothalamus.

FIG. 15 is a series of graphs quantifying signal from whole brain, upper spinal cord (uSC), lower spinal cord (lSC), tail, and paw regions of interest following infusion with 1× aCSF at an acidic or physiologic pH.

FIGS. 16A-16C are a series of line plot profiles of signal (gray value, y-axis) along the ventral surface of the brain (anterior-posterior distribution from olfactory bulb to cerebellum, x-axis) under acidic and physiologic pH conditions following infusion of acidic or physiologic pH 1× aCSF in the lateral ventricle. Each plot corresponds approximately to regions indicated on the fluorescence image at bottom.

FIG. 17 is a graph quantifying signal in regions of interest (dorsal upper spinal cord (duSC), ventral upper spinal cord (vuSC), posterior fossa (PF), and anterior fossa (AF)) under acidic and physiologic pH conditions after co-infusion of 100 nm polystyrene nanoparticles with 1× aCSF into the cisterna magna of healthy mice.

FIGS. 18A-18E. FIG. 18A is a schematic that illustrates a traumatic brain injury (sTBI; single injury)model and repetitive TBI (rTBI; multiple injuries)-induced FTD models. The location of the impact center is shown relative to the brain section (right; asterisk) sampled for histological analysis in this figure (dashed line). FIG. 18B is a schematic timeline for assessing acute outcome after sTBI and chronic outcome in the rTBI-FTD neurodegeneration model (once daily TBI for 5 consecutive days). FIG. 18C is a pair of graphs showing temporal evolution of traumatic axonal injury (left) as assessed by beta amyloid precursor protein (bAPP) staining in the corpus callosum at acute to subacute time points (*P<0.05 versus 48 hour sham; two-way analysis of variance (ANOVA)). Also shown (right) is the composite neurological severity score (#P<0.05 versus pre-trauma; *P<0.05 versus sham; two-way ANOVA). Methods were modified from reference (34). FIG. 18D shows representative micrographs demonstrating cortical neuronal TDP-43 mislocalization (short arrow; scale bar=70 m)) in C9BAC mice after rTBI. NeuN, neuronal nuclei; TDP-43, transactive response DNA binding protein 43 kDa; C9BAC, bacterial artificial chromosome transgenic C9orf72; C9orf72, chromosome 9 open reading frame 72. FIG. 18E shows representative micrographs demonstrating cortical reduction of NeuN-stained neurons (scale bar=120 m) in C9BAC mice after rTBI. NeuN, neuronal nuclei; TDP-43, transactive response DNA binding protein 43 kDa; C9BAC, bacterial artificial chromosome transgenic C9orf72; C9orf72, chromosome 9 open reading frame 72.

FIGS. 19A-19E. FIG. 19A is a diagram that depicts the extent of neuronal loss mediated by the combined risk of rTBI and C9orf72 mutation. Risk was absent (“-”) in non-transgenic sham mice or present (“+”) in C9BAC mice with or without rTBI. Colors indicate % reduction from 0% (white) to 40% (red). Lightning bolt denotes the site of impact delivery. Analyses were done using linear mixed effect models that included group and ROI as fixed covariates, as well as the group×ROI interactions. ***P<0.001 for between hemisphere comparisons; #P<0.05, ##P<0.01, ###P<0.001 for between group comparisons. Squares indicate the approximate region of interest used for quantitative analyses shown in FIGS. 19C-F (no pattern, contralateral; hatched, ipsilateral). FIG. 19B is a diagram illustrating a therapeutic model. Following (rTBI), infusion of a hypertonic aCSF solution generates rapid production of CSF by the choroid plexus, which transiently enhances fluid exchange and clearance of waste from the brain. FIGS. 19C-19E are graphs showing significant loss of NeuN stained neurons (FIG. 19C), increase in Iba-1 stained microglia (FIG. 19D), and TDP-43 mislocalization (FIG. 19E) at 12 months after rTBI in C9BAC mice; this is not seen in non-transgenic (Ntg) and sham controls. Data are mean s.e.m. n=9-12 per group. Iba-1, ionized calcium binding adaptor molecule 1; s.e.m., standard error of the mean.

FIG. 20. Mice were subjected to closed head injury (CHI), then either not injected (“sham”) or received a volume flush (“1×”, 10 μL of isotonic aCSF) or a hypertonic flush (“2×”, 10 μL of 2× hypertonic aCSF). Cytokine levels (G-CSF, CXCL1) were measured in plasma (pg/mL) following treatments.

FIGS. 21A-21C. CHI mice received polystyrene nanoparticle infusions (20, 40, and 100 nm nanoparticle diameters) via intrathecal, cisterna magna administration site (IT-CM) with CFE (CFE; 2×aCSF) or without CFE as a control (Ctr; 1×aCSF). In FIGS. 21A-21B, infusions were delivered at 8 hours after CHI. FIG. 21A. Stereoscopic imaging of the ventral surface of whole brains. Insets denote appearance of vascular trees. FIG. 21B. Brains were sliced to 2 mm coronal sections and imaged with a stereoscope. FIG. 21C. Quantifying total fluorescence in each slice. Nanoparticles were administered IT-CM at 4, 8, or 24 hours after injury; tissue position is shown on the x-axis: 1 (slice 1) is olfactory bulb and tissue position 8 (slice 8) is brainstem/cerebellum.

FIG. 22. CHI mice received polystyrene nanoparticle infusions (data aggregated for 20, 40, and 100 nm nanoparticle diameters) via intrathecal, cisterna magna administration site administration (IT-CM) at 8 hours after CHI, with CFE (CFE) or without CFE as a control. The “Sum of all slices” quantifies the fluorescent signal in the injured and uninjured hemispheres. AU, arbitrary units.

FIG. 23: CHI mice were administered 10 μL of standard (1×aCSF control), low dose CFE (2×aCSF), or high dose CFE (4×aCFE) infusion containing nanoparticles. Top row shows brain tissue was stained with a nuclear stain (DAPI) and for the presence of aquaporin-4 (green). Nanoparticles appeared red. Bottom row shows inverted detection of nanoparticle signal. Dashed lines outline the paths of blood vessels.

FIG. 24. Images show fluorescent nanoparticle (FNP) signal on the ventral surface (underside) of the brain (top panels), with dashed boxes indicating the location of olfactory bulb, lateral vessels, and brainstem images. Animals were treated under the same conditions described for FIG. 23. Arrows indicate the Circle of Willis, which is a network of arteries on the ventral aspect of the brain and serves as an anatomical reference point.

FIG. 25. Images show fluorescent nanoparticle (FNP) signal along the spinal cord (top panels), with dashed boxes indicating the location of cervical and thoraco-lumbar images. Animals were treated under the same conditions described for FIG. 23. Arrows indicate exiting nerve groups.

FIG. 26 shows quantification of experiments represented in FIGS. 24-25.

FIG. 27. Images showing distribution of fluorescent nanoparticles (NPs) under CFE conditions in brains from NSG mice bearing orthotopic medulloblastoma (MB; HD-MB03 cells) exhibiting leptomeningeal metastasis (LM). CFE=CSF flow enhancement (2× aCSF, 10 μL infusion into the CM). Scale bars, 5 mm.

FIGS. 28A-28B. Sagittal view images of the brain from NSG mice bearing orthotopic MB (HD-MB03 cells) exhibiting LM showing 40 nm nucleic acid nanoparticle distribution from the injection site to sites of tumor metastasis under standard infusion (FIG. 28A) and CFE (FIG. 28B) conditions. Scale bars, 5 mm.

FIG. 29. Data quantifying spatial distribution of water-soluble tracer across the brain (left to right on coronal sections) as a result of pH manipulation (pH=4.8 or 7.0) and tonicity manipulation (aCSF=1× or 2×).

FIGS. 30A-30C. Still images from dynamic imaging of non-PEGylated and PEGylated nanoparticles. FIG. 30A. Non-PEGylated (standard) nanoparticles exhibit smooth, unidirectional flow. FIG. 30B. PEGylated nanoparticles show oscillatory movement with entrainment of nanoparticles to each other. FIG. 30C. A reduced concentration of PEGylated nanoparticles shows reduced aggregation and directional flow.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

Headings used in this application are for convenience only and do not affect the interpretation of this application.

### Definitions

As used herein, the term “osmolarity” describes the total concentration of solutes (e.g., sodium, potassium) within a fluid (e.g., CSF or aCSF).

As used herein, the term “isotonic” refers to a condition wherein the total concentration of solutes (e.g., sodium, potassium) within a fluid (e.g., CSF or aCSF) is the same as or approximates an expected (e.g., normal, healthy) physiological osmolarity.

As used herein, the term “hypertonic” refers to a condition wherein the total concentration of solutes (e.g., sodium, potassium) within a fluid (e.g., CSF or aCSF) is higher than an expected (e.g., normal, healthy) physiological osmolarity. In some embodiments, hypertonic solutions are denoted as, e.g., “2×,” meaning the total concentration of solutes (e.g., sodium, potassium) within a fluid (e.g., CSF or aCSF) is about two times the total concentration of solutes (e.g., sodium, potassium) within a fluid (e.g., CSF or aCSF) present in an isotonic condition. In some embodiments, “hypertonic” may involve increasing the concentration of all solutes (e.g., sodium, potassium) within a fluid (e.g., CSF or aCSF) equivalently. In some embodiments, “hypertonic” may involve increasing the concentration of one or more individual solutes (e.g., sodium, potassium) in fluid (e.g., CSF or aCSF) while maintaining the concentration of the other solutes at a physiological level.

As used herein, the term “traumatic brain injury” (TBI) refers to an injury caused by an outside force, e.g., a forceful impact or blast to the head.

As used herein, the term “single TBI” (sTBI) refers to a singular TBI.

As used herein, the terms “recurrent TBI” and “repetitive TBI” (rTBI) refer to multiple TBIs affecting the same individual subject, wherein each TBI occurs at a discrete time.

### General

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., +10%, +5% or +1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours. As used herein, the symbol “±” denotes a range, that is, where a given value is “NIX” a range from N−X to N+X, wherein the range includes the values of N−X and N+X.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

When introducing elements disclosed herein, the articles “a,” “an,” “the,” and “said” are intended to mean that there are one or more of the elements. Further, the one or more elements may be the same or different. Thus, for example, unless the context clearly indicates otherwise, “an agent” includes a single agent, and two or more agents. Further the two or more agents can be the same or different as, for example, in embodiments wherein a first agent comprises a polynucleotide of a first sequence and a second agent comprises a polynucleotide of a second sequence.

As used herein, “increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

As used herein, “increases” also means increases by at least 1-fold, for example, 1-, 2-, 3—, 4-, 5-, 6-, 7-, 8-, 9-, 10-, 15-, 20-, 30-, 40-, 50-, 60-, 70-, 80-, 90-, 100-, 200-, 500-, or 1000-fold or more, for example, as compared to the level of a as compared to the level of a reference standard.

As used herein, “decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99, or 100%, for example, as compared to the level of reference.

As used herein, “decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

As used herein, the term “reference” means a standard or control condition (e.g., untreated with a test agent or combination of test agents). Alternatively, “reference” may refer to a resource, such as an annotated genome, transcriptome, or the like, that is used to assemble, analyze, and/or interpret data.

As used herein, the term “eliminate” means to decrease to a level that is undetectable.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “isolated” refers to a material that is free to varying degrees from components which normally accompany it as found in its native state. “Isolate” denotes a degree of separation from original source or surroundings.

### Central Nervous System (CNS)

As used herein, the term “distribution” can refer to distribution from cerebrospinal fluid (CSF) into parenchyma and/or perivascular space and/or movement from a brain region of interest to the spinal cord and/or changes in the spatial pattern of distribution within a given tissue region. In some embodiments, “distribution” encompasses multiple aspects of how fluid redistributes and multiple potential impacts on nanoparticles.

As used herein, the term “clearance” can refer to movement from a central nervous system (CNS) region of interest into the periphery of the CNS and/or clearance from brain to spinal cord.

As used herein, the term “flow” can refer to production of CSF in various locations within the ventricular system, movement through different parts of the ventricular system, movement from the ventricular system into subarachnoid space, exchange of fluid between tissue compartments (e.g., exchange of interstitial and perivascular fluids), flow from the subarachnoid space into perivascular spaces and along or within neural sheaths, movement from the subarachnoid space into arachnoid granulations (thereby leading to clearance to the periphery), flow from the perivascular spaces into cervical lymphatics, direct reabsorption of CSF or interstitial fluid across capillary walls, and/or transport across the nasal epithelium.

As used herein, the term “parenchyma” refers to the functional tissue of the brain, e.g., brain tissue without blood vessels. The parenchyma comprises, for example, mainly neuronal cells and glial cells. As used herein, the term “perivascular space” refers to fluid-filled space surrounding blood vessels within the central nervous system, e.g., the brain and the spinal cord.

### Subjects

The terms “subject” and “patient” are used interchangeably herein. The term “patient” refers to a human, while the term “subject” may refer to a human or a non-human animal.

As used herein, a “subject” is a vertebrate, including any member of the class mammalia.

As used herein, a “mammal” refers to any mammal including but not limited to human, mouse, rat, sheep, monkey, goat, rabbit, hamster, horse, cow or pig.

A “non-human mammal”, as used herein, refers to any mammal that is not a human.

In some embodiments, the subject is a human.

In some embodiments, the subject has one X chromosome and one Y chromosome. In some embodiments, the subject has two X chromosomes. In some embodiments, the subject has two X chromosomes and one Y chromosome. In some embodiments, the subject has one X chromosome and two Y chromosomes. In some embodiments, the subject is a human male. In some embodiments the subject is human female.

In some embodiments, the subject is at least about 1 month of age, for example, at least about 2, 3, 4, 5, 6, 7, 8, 9, 10, 11, 12, 15, 18 or 21 months of age, or at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years of age. In some embodiments, the subject is about: 1-100, 1-80, 1-60, 1-30, 1-24, 1-20, 1-18, 1-12, 1-10, 1-8, 1-6, 2-100, 2-80, 2-60, 2-30, 2-24, 2-20, 2-18, 2-12, 2-10, 2-8, 2-6, 3-100, 3-80, 3-60, 3-30, 3-24, 3-20, 3-18, 3-12, 3-10, 3-8, 3-6, 4-100, 4-80, 4-60, 4-30, 4-24, 4-20, 4-18, 4-12, 4-10, 4-8, 4-6, 5-100, 5-80, 5-60, 5-30, 5-24, 5-20, 5-18, 5-12, 5-10, 5-8, 6-100, 6-80, 6-60, 6-30, 6-24, 6-20, 6-18, 6-12, 6-10, 8-100, 8-80, 8-60, 8-30, 8-24, 8-20, 8-18, 8-12, 10-100, 10-80, 10-60, 10-30, 10-24, 10-20, 10-18, 12-100, 12-80, 12-38, 12-60, 12-50, 12-40, 12-30, 12-24, 12-20, 12-18, 18-100, 18-80, 18-60, 18-50, 18-40, 18-30, 18-24, 20-100, 20-80, 20-60, 20-50, 20-40, 20-30, 20-25, 30-100, 30-80, 30-60, 30-55, 30-50, 30-45, 30-40, 40-100, 40-80, 40-60, 40-55 or 40-50 years of age. In some embodiments, the subject is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or 100 years of age. In some embodiments, the subject is about 12-38 years of age. In other embodiments, the subject is a fetus. In some embodiments, the subject is a neonatal subject.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older, 75 years of age or older or 90 years of age or older. In some embodiments, the subject is 50 years of age or older. In some embodiments, the subject is a child (i.e., a pediatric subject). In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject is about 2-11, 4-17, 12-18, 18-50, 18-90 or 50-90 years of age.

### Conditions

### Neuroinflammation

In some embodiments of the disclosed invention, a subject has, is suspected to have, or is at risk for having a stroke, neurodegenerative disease, neuroinflammation, cancer affecting a central nervous system (CNS), disease or infection of the CNS, cerebrovascular disease, or a combination thereof.

As used herein, the term “neurodegeneration” refers to a reduction in one or more features, structures, or characteristics of a neuron or neuronal tissue. In some embodiments, neurodegeneration is observed as a pathological reduction in an organism. Those skilled in the art will appreciate that neurodegeneration is associated with certain diseases, disorders and conditions, including those that affect humans. In some embodiments, neurodegeneration may be transient (e.g., as sometimes occurs in association with certain infections and/or chemical or mechanical disruptions); in some embodiments, neurodegeneration may be chronic and/or progressive (e.g., as is often associated with certain diseases, disorders or conditions such as, but not limited to, Parkinson's disease, amyotrophic lateral sclerosis, multiple sclerosis, Huntington's disease, or Alzheimer's disease). In some embodiments, neurodegeneration refers to loss of synapses. In some embodiments, neurodegeneration refers to a reduction in neural tissue relating to a traumatic injury (e.g. exposure to an external force which disrupts the integrity of the neural tissue). In some embodiments, neurodegeneration refers to a reduction in peripheral neural tissue. In some embodiments, neurodegeneration refers to a reduction in central nervous tissue.

Neurodegeneration and neurodegenerative disorders include progressive structural and/or functional loss of nerve cells or neurons in the peripheral nervous system (PNS) and/or central nervous system (CNS). Many degenerative diseases or conditions are known to manifest and/or include axonal and/or synaptic degradation (e.g., Wallerian degeneration) and each of these diseases is suitable for treatment using the compositions and methods disclosed herein. Examples of neurodegenerative diseases that can be treated using the compositions and methods disclosed herein include, but are not limited to, the classes of disease: central nervous system (CNS) disorders, peripheral nervous system (PNS) disorders, trauma-related disorders (including trauma to the head, the spine, and/or the PNS), genetic disorders, metabolic and/or endocrine related disorders (e.g., peripheral neuropathy in diabetes), toxin-related disorders (e.g., peripheral neuropathy induced by toxins (including chemotherapeutic agents)), inflammatory disease, exposure to excess vitamin, vitamin deficiency, and cardiovascular-related disorders (e.g., stroke). Examples of these classes include, but are not limited to, the following diseases and/or causes of disease: Huntington's disease, Alzheimer's disease, Parkinson's disease, Amyotrophic lateral sclerosis (ALS)), axonal abnormalities (e.g., Wallerian degeneration), age-related neurodegeneration (including, for example, dementia), dementia pugilistica (or so called ‘punch drunk syndrome”), shaken baby syndrome, spinal cord injuries (including injuries attributable to stretching, bruising, applying pressure, severing, and laceration), peripheral neuropathy disease or trauma, Friedreich's ataxia, Charcot-Marie-Tooth syndrome, diabetic neuropathy, diabetes mellitus, chronic renal failure, porphyria, amyloidosis, liver failure, hypothyroidism, exposure to certain drugs/toxins (including, for example, vincristine, phenyloin, nitrofurantoin, isoniazid, ethyl alcohol, and/or chemotherapeutic agents, organic metals, heavy metals, fluoroquinolone drugs), excess intake of vitamin B6 (pyridoxine), Guillain-Barre syndrome, systemic lupus erythematosis, leprosy, Sjögren's syndrome, Lyme Disease, sarcoidosis, polyglutamine (so called polyQ) diseases, Kennedy disease, Spinocerebellar ataxia Types 1, 2, 3, 6, 7, and/or 17, non-polyglutamine diseases, vitamin (e.g., vitamin B12 (cyanocobalamin), vitamin A, vitamin E, vitamin B1 (thiamin)) deficiency, exposure to physical trauma (e.g., exposure to compression, pinching, cutting, projectile injuries (e.g., gunshot wound), shingles, malignant disease, HIV, radiation, and chemotherapy.

Examples of neurodegenerative diseases and families or categories of neurodegenerative diseases include, but are not limited to: Parkinson's disease, chronic traumatic encephalopathy (CTE), and lysosomal storage diseases. Further examples of neurodegenerative diseases and families or categories of neurodegenerative diseases include, but are not limited to: Alzheimer's disease, Down syndrome, Lewy body dementia, vascular dementia, frontotemporal dementia/amyotrophic lateral sclerosis, and mixed type dementia. Further examples of neurodegenerative diseases and families or categories of neurodegenerative diseases include, but are not limited to: normal pressure hydrocephalus, Huntington's disease, corticobasal degeneration, traumatic encephalopathy syndrome (e.g., clinical presentation of suspected CTE), prion diseases (such as Creutzfeldt-Jakob disease), progressive supranuclear palsy, ARTAG (aging-related tau astrogliopathy), and LATE (limbic-predominant age-related TDP-43 encephalopathy). Examples of unique forms of vascular disease that are the cause or are related to dementia include, but are not limited to: CADASIL (cerebral autosomal dominant arteriopathy with subcortical infarcts and leukoencephalopathy) and CAA (cerebral amyloid angiopathy). Leukoencephalopathies are a broad group of diseases that affect the brain's white matter.

### Cancer

In some embodiments of the disclosed invention, the subject has, is suspected to have, or is at risk for having a cancer affecting a central nervous system (CNS). The cancer can be a primary cancer (e.g., at a primary tumor site), a metastatic cancer (e.g., comprising one or more metastases of a tumor), or both. In some embodiments, the cancer is associated with leptomeningeal metastases (LM). In some embodiments, the cancer comprises brain cancer, e.g., medulloblastoma (MB). In some embodiments, the cancer comprises a solid tumor.

The examples described herein disclose studies utilizing models of human cancer, including, for example, breast cancer and medulloblastoma. MDA-MB-231, a model of human breast cancer, is a cell line derived from a brain metastasis of a breast adenocarcinoma (see, for example, Knier et al., Preclinical Models of Brain Metastases in Breast Cancer. Biomedicines. 2022 Mar. 13; 10(3):667. doi: 10.3390/biomedicines10030667. PMID: 35327469; PMCID: PMC8945440). MDA-MB-231 cells can be obtained or modified from, for example, the eponymous cell line available from the American Type Culture Collection (Manassas, VA, USA; Catalog No. HTB-26). HD-MB03, a model of human medulloblastoma, is a cell line derived from a metastasized group 3 medulloblastoma in the cerebellum (see, for example, Milde et al., HD-MB03 is a novel Group 3 medulloblastoma model demonstrating sensitivity to histone deacetylase inhibitor treatment. J Neurooncol. 2012 December; 110(3):335-48. doi: 10.1007/s11060-012-0978-1. Epub 2012 Oct. 6. PMID: 23054560). HD-MB03 cells can be obtained or modified from, for example, the eponymous cell line available from Leibniz Institute DSMZ-German Collection of Microorganisms and Cell Cultures GmbH (Braunschweig, Germany; Catalog No. ACC 740). Med-411FH, also a model of human medulloblastoma, is a cell line that can be obtained or modified from, for example, the eponymous cell line available from the Brain Tumor Resource Lab at Seattle Children's (Seattle, WA, USA).

As will be readily understood by those of skill in the art, the methods and compositions disclosed herein can be applied or administered to a subject that has, is suspected to have, or is at risk for having a cancer not limited to the model cancers utilized in the disclosed examples. For example, the subject can have, be suspected to have, or be at risk for having cancer cells present in the brain, spinal cord, or both.

### Injury

In some embodiments, a subject has or is suspected to have a traumatic brain injury (TBI), a traumatic spinal cord injury, or both. In some embodiments, a traumatic brain injury is a closed head injury (e.g., as described in Kahriman et al. 2021 (38)), a concussion injury (e.g., as described in Bouley et al. 2019 (32)), a repetitive traumatic brain injury (e.g., as described in Kahriman et al. 2022 (112) Kahriman et al. 2021 (33), Dogan et al. 2023 (85), or Dogan et al. 2025 (113)), an injury comprising axonal injury (e.g., as described in Henninger et al. 2016 (34)), or a combination of the foregoing.

In some embodiments, a traumatic brain injury or a traumatic spinal cord injury comprises one or more injuries, e.g., two, three, four, five, or more injuries. In some embodiments, a repetitive traumatic brain injury or a repetitive traumatic spinal cord injury comprises two or more injuries, e.g., three, four, five, or more injuries. In some embodiments, a traumatic brain injury or a traumatic spinal cord injury is induced in a model organism by means of falling weight, e.g., about 25 grams to about 50 grams of falling weight.

### Compositions

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C1-C4)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual.

In some embodiments, an agent disclosed herein (e.g., hypertonic aCSF) is in a form of a pharmaceutical composition, or a pharmaceutically acceptable salt thereof. A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a pharmaceutical composition disclosed herein is formulated as a solution, e.g., an infusion solution. In some embodiments, an infusion solution is hypertonic. In some embodiments, a hypertonic infusion solution comprises 2× aCSF. In some embodiments, a hypertonic infusion solution induces CFE. In some embodiments, a hypertonic infusion solution is a colloid. In some embodiments, a hypertonic infusion solution comprises 2× aCSF and one or more functional agents, e.g., nanoparticles, therapeutic agents, and the like.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods disclosed herein further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO (N-(Hydroxyethyl)piperazine-N′-2-hydroxypropanesulfonic acid) and HEPES (4-(2-hydroxyethyl)-1-piperazineethanesulfonic acid).

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., hypertonic aCSF) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

In some embodiments, a composition comprises a solubilizing agent, e.g., a colloid, e.g., a cyclodextrin. In some embodiments, a composition comprises a therapeutic agent, an excipient, a diluent, a carrier, an adjuvant, or a combination thereof, having a dimension up to about 1 μm, up to about 500 nm, up to about 400 nm, up to about 300 nm, up to about 200 nm, up to about 100 nm, up to about 10 nm, up to about 5 nm, or up to about 1 nm. In some embodiments, a composition comprises a therapeutic agent, an excipient, a diluent, a carrier, an adjuvant, or a combination thereof, having a dimension of about 1 μm, of about 500 nm, of about 400 nm, of about 300 nm, of about 200 nm, of about 100 nm, of about 10 nm, of about 5 nm, or of about 1 nm. In some embodiments, a composition comprises a therapeutic agent, an excipient, a diluent, a carrier, an adjuvant, or a combination thereof, having a dimension up to about 5 nm. In some embodiments, a composition comprises a therapeutic agent, an excipient, a diluent, a carrier, an adjuvant, or a combination thereof, having a dimension of about 5 nm.

In some embodiments, a composition comprises an injection solution. In some embodiments, an injection solution comprises artificial cerebrospinal fluid (aCSF) or phosphate buffered solution (PBS). In some embodiments, an injection solution comprises a hypertonic solution. In some embodiments, a hypertonic solution comprises a hypertonic artificial CSF (aCSF) or a hypertonic phosphate buffered solution (PBS).

In some embodiments, an injection solution, a hypertonic solution, a hypertonic aCSF, a hypertonic PBS, or a combination thereof has a total osmolarity that is higher than a) the total osmolarity of the CSF of a subject, orb) an expected, normal, and/or healthy total osmolarity of the CSF of a subject or the species of the subject.

As used herein, the term “osmolarity” means the number of osmoles (Osm) of solute particles (e.g., sodium (Na⁺), potassium (K⁺)) per liter (L) of solution (e.g., CSF or aCSF). As used herein, “osmolarity” may describe the total osmolarity of a fluid or the osmolarity of one or more solute particles (e.g., sodium, potassium) within a fluid (e.g., CSF or aCSF). As used herein, the term “total osmolarity” describes the total concentration of all solute particles or of all of one or more specified types of solute particles (e.g., sodium, potassium) within a fluid (e.g., CSF or aCSF).

“Osmolarity” and “tonicity” and related terms may be used interchangeably herein. “Osmolarity” is commonly used in the art to refer to a concentration of all solute particles in a solution, while “tonicity” is commonly used in the art to refer to a concentration of only solute particles that cannot freely diffuse (e.g., that require active transport) across a barrier (e.g., a cell membrane). As used herein, tonicity refers to a concentration of solute particles in a solution wherein one or more of the solute particles cannot freely diffuse across the blood-brain barrier (BBB). For example, as used herein, the term “isotonic” refers to a condition wherein the concentration of solute particles (e.g., sodium, potassium) within a fluid (e.g., CSF or aCSF) is the same as or approximates an expected (e.g., normal, healthy) physiological concentration of solute. As used herein, the term “hypertonic” refers to a condition wherein the concentration of solute particles (e.g., sodium, potassium) within a fluid (e.g., CSF or aCSF) is higher than an expected (e.g., normal, healthy) physiological concentration of solute. In some embodiments, hypertonic solutions are denoted as, e.g., “2×,” meaning the total concentration of solutes (e.g., sodium, potassium) within a fluid (e.g., CSF or aCSF) is about two times the total concentration of solutes (e.g., sodium, potassium) within a fluid (e.g., CSF or aCSF) present in an isotonic condition.

For the purposes of describing the composition of injection solutions disclosed herein, concentrations of solute are expressed in terms of osmolarity (e.g., milliosmoles per liter (mOsm/L). For example, an injection solution disclosed herein may be described as a) being hyperosmolar and/or comprising hyperosmolar concentrations of one or more solute particles, and b) hypertonic. A hypertonic injection solution, upon administration to the CSF, is expected to provoke a physiological response to the hypertonicity to restore homeostatic osmolarity conditions to the CSF.

In some embodiments, an injection solution, a hypertonic solution, a hypertonic aCSF, a hypertonic PBS, or a combination thereof has an osmolarity of a component (e.g., sodium (Na⁺); e.g., one or more components) that is higher than a) the osmolarity of said component in the CSF of a subject, or b) an expected, normal, and/or healthy osmolarity of said component in the CSF of a subject or the species of the subject.

In some embodiments, an injection solution, a hypertonic solution, a hypertonic aCSF, a hypertonic PBS, or a combination thereof has an osmolarity of one or more components selected from sodium (Na⁺), potassium (K⁺), Calcium (Ca²⁺), magnesium (Mg²⁺), chloride (Cl⁻), bicarbonate (HCO₃⁻), and phosphate (PO₄³⁻), that is higher than a) the osmolarity of said component in the CSF of a subject, or b) an expected, normal, and/or healthy osmolarity of said component in the CSF of a subject or the species of the subject.

In some embodiments, an injection solution, a hypertonic solution, a hypertonic aCSF, a hypertonic PBS, or a combination thereof has a total osmolarity that is about 1.1 times (e.g., 1.1×, 110%, or 10% higher than) to about 8 times (e.g., 8×, 800%, or 700% higher than) a) the total osmolarity of the CSF of a subject, or b) an expected, normal, and/or healthy total osmolarity of the CSF of a subject or the species of the subject.

In some embodiments, an injection solution, a hypertonic solution, a hypertonic aCSF, a hypertonic PBS, or a combination thereof has a total osmolarity that is about 1.1 times (e.g., 1.1×, 110%, or 10% higher than) to about 4 times (e.g., 4×, 400%, or 300% higher than) a) the total osmolarity of the CSF of a subject, or b) an expected, normal, and/or healthy total osmolarity of the CSF of a subject or the species of the subject.

In some embodiments, an injection solution, a hypertonic solution, a hypertonic aCSF, a hypertonic PBS, or a combination thereof has a total osmolarity that is about 1.5 times (e.g., 1.5×, 150%, or 50% higher than) to about 2 times (e.g., 2×, 200%, or 100% higher than) a) the total osmolarity of the CSF of a subject, or b) an expected, normal, and/or healthy total osmolarity of the CSF of a subject or the species of the subject.

In some embodiments, an injection solution, a hypertonic solution, a hypertonic aCSF, or a combination thereof for administration to a mouse comprises:
a) about 100 to about 200 mOsm Na⁺,
b) about 2 to about 4 mOsm K⁺,
c) about 1 to about 2.5 mOsm Ca²⁺,
d) about 1.5 to about 3 mOsm Mg²⁺,
e) about 100 to about 200 mOsm Cl⁻,
f) about 18 to about 27 mOsm HCO₃⁻, or
g) any combination of the foregoing.

In some embodiments, an injection solution, a hypertonic solution, a hypertonic aCSF, or a combination thereof for administration to a mouse comprises:
a) about 125 mOsm Na⁺,
b) about 3 mOsm K⁺,
c) about 2.3 mOsm Ca²⁺,
d) about 2.2 mOsm Mg²⁺,
e) about 134 mOsm Cl⁻,
f) about 23 mOsm HCO₃⁻, or
g) any combination of the foregoing.

In some embodiments, an injection solution, a hypertonic solution, a hypertonic aCSF, or a combination thereof for administration to a mouse has a pH of about 7.3.

### Administration

In some embodiments, an agent disclosed herein (e.g., hypertonic aCSF) is delivered using controlled or sustained-release delivery systems (e.g., capsules, biodegradable matrices, intrathecal pumps). Example delayed-release delivery systems for drug delivery that would be suitable for administration of a composition described herein are described in U.S. Pat. Nos. U.S. Pat. No. 5,990,092 (issued to Walsh); U.S. Pat. No. 5,039,660 (issued to Leonard); U.S. Pat. No. 4,452,775 (issued to Kent); and U.S. Pat. No. 3,854,480 (issued to Zaffaroni), the entire teachings of which are incorporated herein by reference. Example biodegradable matrices are discussed in Li et al. (Li, C., Guo, C., Fitzpatrick, V. et al. Design of biodegradable, implantable devices towards clinical translation. Nat Rev Mater 5, 61-81 (2020). https://doi.org/10.1038/s41578-019-0150-z).

In some embodiments, a method disclosed herein comprises administering to the subject two or more agents, for example, 2, 3, 4, or 5 or more agents. In some embodiments, the two or more agents are administered together. In other embodiments, the two or more agents are administered separately, e.g., sequentially. In some embodiments, two or more agents are administered in the same composition, e.g., in an infusion solution. In some embodiments, two or more agents are administered in different compositions.

In some embodiments, a method disclosed herein comprises administering an infusion solution to a subject. In some embodiments, the infusion solution is administered directly to the central nervous system (CNS) of a subject. In some embodiments, the infusion solution is administered to a subject by an intracerebroventricular (ICV, i.e., through the parenchyma into a lateral ventricle), intraparenchymal (IP), or intrathecal (IT; e.g., IT-lumbar (IT-L, i.e., through the lumbar cistern) or IT-cisternal (IT-C)) route. Examples of administering an infusion solution directly to the CNS of a subject are described by Vuillemenot et al. (Vuillemenot B R, Korte S, Wright T L, Adams E L, Boyd R B, Butt M T. Safety Evaluation of CNS Administered Biologics-Study Design, Data Interpretation, and Translation to the Clinic. Toxicol Sci. 2016 July; 152(1):3-9), the entire teachings of which are incorporated herein by reference.

In some embodiments, direct administration to the CNS comprises a bolus injection or infusion. As used herein, the term “bolus” refers to a large volume administered within a timeframe. The term “slow bolus” generally refers to a relatively large volume administered at a relatively slow rate. Examples of slow bolus infusion volumes and times across species are shown below.

| | | |
|---|---|---|
| Mouse | Sheep | Human |

| | | | | | |
|---|---|---|---|---|---|
| CSF Volume | 40 | μL | 20 mL | 125 | mL |
| Maximum volume | 20 | μL | 10 mL | 25 | mL |
| Typical volume | 10 | μL | | 1-12 | mL |

| | | |
|---|---|---|
| Typical time | 20-80 | sec |

Tolerable volumes of an infusion solution for direct administration to the CNS are understood to range from about 20% to about 50% of the CSF volume of a subject (see table above). As used herein, “tolerable” means that a subject is likely to receive a treatment or intervention (e.g., administration of an infusion solution) without experiencing permanent side effects.

In some embodiments, a bolus volume is up to about 20% of the CSF volume of a subject. In some embodiments, a bolus volume is up to about 50% of the CSF volume of a subject.

In some embodiments, a catheter is implanted (e.g., into the lumbar space) to facilitate direct administration to the CNS. In some embodiments, a dosing port is implanted (e.g., into the lumbar space) to facilitate direct administration to the CNS. In some embodiments, an intrathecal pump or implanted intrathecal drug delivery system (IDDS) is implanted into the lumbar space to facilitate direct administration to the CNS.

Tolerable and/or safe rates of infusion (e.g., of an infusion solution) directly into the CNS (e.g., by an intrathecal route) are understood to vary across species. In some embodiments, a rate of infusion of about ≤0.12 mL/min is considered tolerable to rats and about 2 mL/min is considered tolerable to non-human primates.

In some embodiments, posture of a subject (e.g., supine or sitting upright) may be adjusted to facilitate administration. Without being bound by any theory, a safe, tolerable infusion rate may be, in part, impacted by the intracranial pressure associated with a certain posture of a subject.

In some embodiments, an infusion solution can be administered to a subject in need thereof within a window of time after the subject has sustained a traumatic brain injury and/or a traumatic spinal cord injury. In some embodiments, an infusion solution is administered within about 48 hours, within about 24 hours, within about 8 hours, or within about 4 hours of a traumatic brain injury and/or a traumatic spinal cord injury. In some embodiments, an infusion solution is administered within about 8 hours of a traumatic brain injury and/or a traumatic spinal cord injury. In some embodiments, an infusion solution is administered within about 4 hours of a traumatic brain injury and/or a traumatic spinal cord injury. In some embodiments, an infusion solution is administered within about 48 hours, within about 24 hours, within about 8 hours, or within about 4 hours of a recurrent traumatic brain injury and/or a recurrent traumatic spinal cord injury.

In some embodiments, administration of an infusion solution disclosed herein (e.g., comprising 2× or 4× aCSF) can enhance delivery of a colloid, a constituent thereof, or both to a location in the central nervous system, e.g., the parenchyma, perivascular space, olfactory bulb, lateral vessels of the brain, the brainstem, the cervical region of the spinal cord, the thoraco-lumbar region of the spinal cord, exiting nerve groups of the spinal cord, and the like.

### Functional Agents

As used herein, the term “therapeutic agent” refers to a biocompatible agent for the treatment and/or prevention of a disease. Examples of therapeutic agents include, but are not limited to: a modified or unmodified DNA, RNA, protein, peptide, lipid, nanoparticle, or liposome; a nanoparticle, viral vector, small molecule, large molecule, aptamer, or chemotherapeutic.

As used herein, an “infusion solution” refers to an artificial cerebrospinal fluid (aCSF) that, in some embodiments, is hypertonic. In some embodiments, an infusion solution further comprises a functional agent. In some embodiments, a functional agent comprises a nanoparticle. In some embodiments, a functional agent comprises a therapeutic agent. In some embodiments wherein the infusion solution comprises a therapeutic agent, the infusion solution further comprises an excipient, diluent, carrier, or adjuvant. In some embodiments, the infusion solution comprises a cyclodextrin. In some embodiments, an infusion solution is administered in combination with an additional therapy. In some embodiments, the additional therapy is co-administered with the infusion solution. In some embodiments, the infusion solution and the additional therapy are administered separately. In some embodiments, the infusion solution and the additional therapy are administered simultaneously. In some embodiments, the infusion solution and the additional therapy are administered sequentially.

In some embodiments, an infusion solution comprises a functional agent comprising a nanoparticle. In some embodiments, a nanoparticle comprises a fluorescent nanoparticle (FNP). In some embodiments, a nanoparticle comprises polystyrene, a β-cyclodextrin-poly (β-amino ester) network (CDN₅), gold, polyester, a nucleic acid, poly(lactic-co-glycolic) acid (PLGA), or any combination of the foregoing. In some embodiments, a nanoparticle further comprises a label (e.g., a fluorescent label, a radiolabel, or both). In some embodiments, a nanoparticle comprises FLUOSPHERES® carboxylate-modified fluorescent microspheres (Invitrogen by Thermo Fisher Scientific, Waltham, MA, USA), wherein the microspheres are coated with a hydrophilic polymer containing carboxylic acids. In general, the terms “polystyrene nanoparticles” and “FLUOSPHERES®” are used interchangeably herein. In some embodiments, a nanoparticle comprises carboxylic acids, wherein the carboxylic acids can be used for covalent attachment of ligands and the like. In some embodiments, a nanoparticle has a negative charge. Without being bound by theory, a negatively charged nanosparticle may be advantageous as part of a hypertonic infusion solution (e.g., a colloid) because negatively charged suspended particles of colloids repel each other, thereby promoting distribution of the suspended particles (e.g., functional agents) through the perivascular spaces and parenchyma of the brain and/or spinal cord.

In some embodiments, a method disclosed herein further comprises administering to a subject at least one additional (e.g., second) agent (e.g., a chemotherapy), local treatment (e.g., surgery and/or radiotherapy), or any combination thereof. In some embodiments, a method further comprises administering to a subject an additional (e.g., second) agent. In some embodiments, a method further comprises administering to a subject a local treatment. In some embodiments, a method further comprises administering to a subject a chemotherapy. In some embodiments, a method further comprises administering to a subject a radiation therapy.

### Antibodies and Related Agents

As used herein, an “antibody” is any immunoglobulin polypeptide, or fragment thereof, having immunogen binding ability.

In some embodiments, an “antibody” refers to a whole antibody, an intact antibody, or an antigen-binding fragment of an antibody. As used herein, the term “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. The antibody can be of any species, such as a murine antibody, a human antibody or a humanized antibody. In some embodiments, the term “antibody” refers to a full-length antibody comprising two heavy (H) chains and two light (L) chains inter-connected by disulfide bonds or multimers thereof (for example, IgM). Each heavy chain comprises a heavy chain variable region (V_{H}) and a heavy chain constant region. Each light chain comprises a light chain variable region (V_{L}) and a light chain constant region.

The term “antigen-binding fragment” refers to a portion of an immunoglobulin molecule (e.g., an antibody) that retains the antigen binding properties of the parental full-length antibody. Non-limiting examples of antigen-binding fragments include a V_{H} region, a V_{L} region, an Fab fragment, an F(ab′)₂ fragment, an Fd fragment, an Fv fragment, and a domain antibody (dAb) consisting of one V_{H} domain or one V_{L} domain, etc. V_{H} and V_{L} domains may be linked together via a synthetic linker to form various types of single-chain antibody designs in which the V_{H}/V_{L} domains pair intramolecularly, or intermolecularly in those cases when the V_{H} and V_{L}domains are expressed by separate chains, to form a monovalent antigen binding site, such as single chain Fv (scFv) or diabody.

The term “bispecific antibody” refers to an antibody capable of binding two distinct antigens simultaneously via two distinct antigen binding sites.

As used herein, a “fragment” is a portion of a polypeptide or nucleic acid molecule. This portion contains in example embodiments, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, “antibody-therapeutic conjugate” or “ATC” refers to an antibody covalently bonded to a therapeutic agent. As used herein, “antibody-drug conjugate” or “ADC” refers to an antibody covalently bonded to a therapeutic drug, such as a small molecule (e.g. SN-38).

As used herein, an “agonist” is an agent that causes an increase in the expression or activity of a target gene or protein, respectively. An agonist can bind to and activate its cognate receptor in some fashion, which directly or indirectly brings about this physiological effect on the target gene or protein.

As used herein, an “inhibitor” is an agent that causes a decrease in the expression or activity of a target gene or protein, respectively. An “antagonist” can be an inhibitor but is more specifically an agent that binds to a receptor, which in turn decreases or eliminates binding by other molecules.

### Genes, Transcripts, Proteins, and -Omics

In general, as used herein, italicized symbols indicate genes or transcripts and non-italicized symbols indicate proteins. In general, as used herein, all-capital symbols indicate human origin; and symbols with only the first letter capitalized indicate mouse or other animal origin. As used herein, gene, transcript, and protein symbols may utilize lowercase and uppercase letters interchangeably where species information is provided by context, or where the referent gene, transcript, or protein is analogous across multiple species. As used herein, gene and transcript symbols may be italicized or non-italicized, with information on the type of nucleic acid provided by context.

As used herein, the term “functionally express” refers to the ability of a cell to express an RNA and/or protein at a level, or within a range, that is known in the art to correlate with an identity and/or function of a cell. In some embodiments, functional expression is of a transcript and/or a protein. In some embodiments, functional expression, or the lack thereof, is detected by quantitative polymerase chain reaction (qPCR), RNA sequencing (RNA-seq), flow cytometry, mass cytometry, immunofluorescence (IF), immunohistochemistry (IHC), immunocytochemistry (ICC), in situ hybridization (ISH), fluorescent in situ hybridization (FISH), or other methods or techniques capable of detecting an RNA, a protein, or a portion thereof. In some embodiments, detection of RNA is an acceptable surrogate for protein detection where protein detection has been validated and/or a reasonable expectation of successful translation exists.

As used herein, the term “differentially expressed” refers to a gene, RNA, or protein that is expressed at different levels in two or more groups. A group may be, for example, a cell, a population of cells, or a tissue, and/or a pool of cells or tissues from more than one organism. As used herein, a group reflects an experimental condition, for example, a particular genotype or treatment with a drug. As used herein, the term “differentially expressed gene” refers a gene that is differentially expressed or to the transcriptional product (e.g., mRNA) of a gene that is differentially expressed.

### Model Organisms

As used herein, the terms “mouse strain” and “mouse line” refer to mice that are bred specifically to maintain a certain genotype, which may be wild-type or contain one or more mutations (e.g., substitution, insertion, deletion, early stop codon), chromosomal rearrangements (e.g., translocation, duplication, inversion), transgenes (i.e., a sequence from a different species of organism), knocked-in (i.e., inserted) sequences, knocked-out (i.e., deleted) sequences, floxed sequences (i.e., a sequence flanked by loxP sites), and the like.

Multiple mouse strains may be used to create the strains cited and used herein, or functional equivalents thereto.

An example mouse line used in examples disclosed herein is a NOD scid gamma (NSG) mouse line. NSG mice exhibit a severe combined immune deficient (scid) phenotype attributed to a mutation in the DNA repair complex protein gene, “protein kinase, DNA activated, catalytic polypeptide” (Prkdc) and a null mutation on the “interleukin 2 receptor, gamma chain” gene (Il2rg). NSG mice can be obtained or modified from, for example, NOD.Cg-Prkdc^{scid} I12reg^{tmlWjl}SzJ mice (NSG®; The Jackson Laboratory, Bar Harbor, ME, USA; Research Resource Identifier (RRID):IMSR_JAX: 005557). NSG mice have a non-obese diabetic (NOD) genetic background. NOD mice are discussed in Makino et al. (Breeding of a non-obese, diabetic strain of mice. Jikken Dobutsu. 1980 January; 29(1):1-13. doi: 10.1538/expanim1978.29.1_1. PMID: 6995140). The severe immunodeficiency of NSG mice permits engraftment of xenogeneic cells or tissues, e.g., ID-MB03 cells.

As will be readily understood by those of skill in the art, the methods and compositions disclosed herein can be applied or administered to subjects not limited to the model organisms utilized in the disclosed examples (e.g., to other model organisms or animal or human subjects).

Other definitions appear in context throughout this disclosure.

### Abbreviations and Terminology

C9BAC: bacterial artificial chromosome transgenic C9orf72
C9orf72: chromosome 9 open reading frame 72
CFE: cerebrospinal fluid (CSF) flow enhancement
CSF flush: an alternative term for CFE
F-18: fluoride ion (¹⁸F, radiofluorine), a small, water-soluble molecule used to track fluid movement
FLUOSPHERES® (FS): commercially available polystyrene nanoparticles
Iba-1: ionized calcium binding adaptor molecule 1
MDA-MB-231: breast cancer cell line (example of metastasis in tumor types other than medulloblastoma)
NANPs: nucleic acid nanoparticles, often referred to as DNA origami, can also comprise other nucleic acids
NeuN: neuronal nuclei
PEG: poly(ethylene) glycol
PSNP: polystyrene nanoparticles
TDP-43: transactive response DNA binding protein 43 kDa

### Example Embodiments

### Example Therapeutic Methods and Compositions

In one aspect, the disclosure herein provides a method for enhancing delivery of a therapeutic agent to a parenchyma or perivascular space of a subject in need thereof, said method comprising administering an infusion solution to a cerebrospinal fluid (CSF) of the subject, wherein the infusion solution comprises a hypertonic solution and the therapeutic agent. In some embodiments, administering an infusion solution to a cerebrospinal fluid (CSF) of the subject leads to CSF flow enhancement (CFE; i.e., a CSF flush), thereby enhancing delivery of a therapeutic agent to the parenchyma and/or perivascular space.

In another aspect, the disclosure herein provides a method for treating a subject in need thereof, said method comprising administering an infusion solution to a cerebrospinal fluid (CSF) of the subject, wherein the infusion solution comprises a hypertonic solution.

In another aspect, the disclosure herein provides a method for enhancing cerebrospinal fluid distribution in a subject in need thereof, said method comprising administering an infusion solution to a cerebrospinal fluid (CSF) of the subject, wherein the infusion solution comprises a hypertonic solution.

In another aspect, the disclosure herein provides a method for reducing an abnormal protein deposit in a subject in need thereof, said method comprising administering an infusion solution to a cerebrospinal fluid (CSF) of the subject, wherein the infusion solution comprises a hypertonic solution.

In another aspect, the disclosure herein provides a method for stimulating a choroid plexus (CP) to produce cerebrospinal fluid (CSF) in a subject in need thereof, said method comprising administering an infusion solution to the CSF of the subject, wherein the infusion solution comprises a hypertonic solution.

In another aspect, the disclosure herein provides a method for stimulating production of a cerebrospinal fluid (CSF) in a subject in need thereof, said method comprising administering an infusion solution to the CSF of the subject, wherein the infusion solution comprises a hypertonic solution.

In another aspect, the disclosure herein provides a method for treating a disease or injury of the central nervous system (CNS) in subject in need thereof, said method comprising administering an infusion solution to a cerebrospinal fluid (CSF) of the subject, wherein the infusion solution comprises a hypertonic solution.

In some embodiments, the hypertonic solution has an osmolarity at least about 1.1 times (1.1×) an osmolarity of the CSF of the subject.

In some embodiments, the hypertonic solution has an osmolarity of up to about 8 times (8×) an osmolarity of the CSF of the subject.

In some embodiments, the hypertonic solution comprises one or more electrolytes selected from sodium (Na⁺), potassium (K⁺), Calcium (Ca²⁺), magnesium (Mg²⁺), chloride (Cl⁻), and biocarbonate (HCO₃), wherein an osmolarity of each of the one or more electrolytes is higher than an osmolarity of said electrolytes in the CSF of the subject.

In some embodiments, the subject is a rat and the hypertonic solution has an osmolarity of: up to about 2640 mOsm, about 363 mOsm to about 1320 mOsm, or about 495 mOsm to about 660 mOsm.

In some embodiments, the subject is a human and the hypertonic solution has an smolarity of: up to about 2240 mOsm, about 308 mOsm to about 1120 mOsm, or about 420 mOsm to about 560 mOsm.

In some embodiments, the subject is a mouse and the hypertonic solution has an osmolarity of: up to about 2320 mOsm, about 319 mOsm to about 1160 mOsm, or about 435 mOsm to about 580 mOsm.

In some embodiments, the hypertonic solution comprises: about 100 to about 200 mOsm Na⁺, about 2 to about 4 mOsm K⁺, about 1 to about 2.5 mOsm Ca²⁺, about 1 to about 3 mOsm Mg²⁺, about 100 to about 200 mOsm Cl⁻, about 18 to about 27 mOsm HCO₃⁻, or any combination of the foregoing.

In some embodiments, the hypertonic solution comprises: about 125 mOsm Na⁺, about 3 mOsm K⁺, about 2.3 mOsm Ca²⁺, about 2.2 mOsm Mg²⁺, about 134 mOsm Cl⁻, about 23 mOsm HCO₃⁻, or any combination of the foregoing.

In some embodiments, the hypertonic solution comprises: about 137 to about 1600 mOsm Na⁺, about 3 to about 25 mOsm K⁺, about 1 to about 16 mOsm Ca²⁺, about 1 to about 9 mOsm Mg²⁺, about 124 to about 1072 mOsm Cl⁻, about 25 to about 208 mOsm HCO₃⁻, or any combination of the foregoing.

In some embodiments, the hypertonic solution comprises: a total osmolarity of about 1.1 times (1.1×) to about 8 times (8×) a total osmolarity of the CSF of the subject, an osmolarity of Na⁺ about 1.1 times (1.1×) to about 8 times (8×) an osmolarity of Na⁺ in the CSF of the subject, an osmolarity of K⁺ about 1.1 times (1.1×) to about 8 times (8×) an osmolarity of K⁺ in the CSF of the subject, an osmolarity of Ca²⁺ about 1.1 times (1.1×) to about 8 times (8×) an osmolarity of Ca²⁺ in the CSF of the subject, an osmolarity of Mg²⁺ about 1.1 times (1.1×) to about 8 times (8×) an osmolarity of Mg²⁺ in the CSF of the subject, an osmolarity of Cl⁻ about 1.1 times (1.1×) to about 8 times (8×) an osmolarity of Cl⁻ in the CSF of the subject, an osmolarity of HCO₃⁻ about 1.1 times (1.1×) to about 8 times (8×) an osmolarity of HCO₃⁻ in the CSF of the subject, or any combination of the foregoing.

In some embodiments, a hypertonic solution disclosed herein comprises about 1.1 times (1.1×) to about 8.8 times (8.8×) the total osmolarity or the individual osmolarity of any solute compared to a physiological CSF of a subject, an artificial CSF (aCSF), or another isotonic solution, such as PBS. For example, a total osmolarity or an osmolarity of a solute (e.g., an electrolyte, e.g., sodium (Na⁺), potassium (K⁺), Calcium (Ca²⁺), magnesium (Mg²⁺), chloride (Cl⁻), and biocarbonate (HCO₃)), may be about the osmolarity set forth below (values shown in mOsm).

| | | Total | | | | | | |
|---|---|---|---|---|---|---|---|---|
| | Hypertonicity | Osmolarity | Na⁺ | K⁺ | Ca²⁺ | Mg²⁺ | Cl⁻ | HCO₃⁻ |
| Human | 1.1x | 308 | 154 | 3.146 | 1.254 | 1.21 | 124.3 | 25.63 |
| CSF | 1.5x | 420 | 210 | 4.29 | 1.71 | 1.65 | 169.5 | 34.95 |
| | 2x | 560 | 280 | 5.72 | 2.28 | 2.2 | 226 | 46.6 |
| | 4x | 1120 | 560 | 11.44 | 4.56 | 4.4 | 452 | 93.2 |
| | 8x | 2240 | 1120 | 22.88 | 9.12 | 8.8 | 904 | 186.4 |
| Mouse | 1.1x | 319 | 220 | 3.41 | 1.32 | 0.99 | 138.6 | |
| CSF | 1.5x | 435 | 300 | 4.65 | 1.8 | 1.35 | 189 | |
| | 2x | 580 | 400 | 6.2 | 2.4 | 1.8 | 252 | |
| | 4x | 1160 | 800 | 12.4 | 4.8 | 3.6 | 504 | |
| | 8x | 2320 | 1600 | 24.8 | 9.6 | 7.2 | 1008 | |
| Mouse | 1.1x | 341 | 137.5 | 3.3 | 2.2 | 1.1 | 147.4 | 28.6 |
| aCSF | 1.5x | 465 | 187.5 | 4.5 | 3 | 1.5 | 201 | 39 |
| | 2x | 620 | 250 | 6 | 4 | 2 | 268 | 52 |
| | 4x | 1240 | 500 | 12 | 8 | 4 | 536 | 104 |
| | 8x | 2480 | 1000 | 24 | 16 | 8 | 1072 | 208 |
| PBS | 1.1x | 341 | 137.5 | 3.3 | 2.2 | 1.1 | 147.4 | |
| | 1.5x | 465 | 187.5 | 4.5 | 3 | 1.5 | 201 | |
| | 2x | 620 | 250 | 6 | 4 | 2 | 268 | |
| | 4x | 1240 | 500 | 12 | 8 | 4 | 536 | |
| | 8x | 2480 | 1000 | 24 | 16 | 8 | 1072 | |

In some embodiments, the hypertonic solution has an osmolarity of about 308 to about 2480.

In some embodiments, the hypertonic solution has a pH of about 6 to about 9.

In some embodiments, the hypertonic solution comprises a hypertonic artificial CSF (aCSF) or a hypertonic phosphate buffered solution (PBS).

In some embodiments, the infusion solution further comprises a therapeutic agent.

In some embodiments, the method further comprises the step of administering a therapeutic agent to the subject before, during, or after administering the infusion solution to the subject.

In some embodiments, the therapeutic agent (e.g., the therapeutic agent in the hypertonic solution and/or the therapeutic being administered to the subject before, during, or after administration of the infusion solution) comprises a nanoparticle, DNA, RNA, protein, peptide, lipid, liposome, viral vector, small molecule, large molecule, aptamer, chemotherapeutic, or a combination thereof. In some embodiments, the chemotherapeutic comprises methotrexate, thiotepa, vincristine, cisplatin, cytarabine, cyclophosphamide, or a combination of the foregoing.

In some embodiments, nucleic acid nanoparticles (NANPs) are about 30-50 nm. In some embodiments, the therapeutic agent comprises an analgesic.

In some embodiments, the DNA comprises a genomic DNA (gDNA), complementary DNA (cDNA), oligonucleotide, antisense oligonucleotide (ASO), or a combination thereof.

In some embodiments, the RNA comprises an antisense RNA (asRNA), short interfering RNA (siRNA), oligonucleotide, antisense oligonucleotide (ASO), or a combination thereof.

In some embodiments, the protein comprises an antibody.

In some embodiments, the antibody comprises an immunoglobulin G (IgG).

In some embodiments, the viral vector is an adeno-associated virus (AAV) vector.

In some embodiments, the nanoparticle comprises polystyrene, polyester, a nucleic acid, or poly(lactic-co-glycolic) acid (PLGA).

In some embodiments, the nanoparticle comprises a 20 nm polystyrene sphere, a 40 nm polystyrene sphere, a 100 nm polystyrene sphere, or a polyester sphere.

In some embodiments, the nanoparticle or a portion thereof is spherical, cylindrical, conical, torpedo-shaped, deformable, or a combination thereof.

In some embodiments, an aspect ratio of the nanoparticle is about 1:1 to 1:50.

In some embodiments, a dimension of the therapeutic agent is up to about 1 micron, for example, about 500 nm.

In some embodiments, a dimension of the therapeutic agent is about 100 to 400 nm.

In some embodiments, a dimension of the therapeutic agent is about 200 to 300 nm.

In some embodiments, a dimension of the therapeutic agent is up to about 100 nm.

In some embodiments, the therapeutic agent is charged.

In some embodiments, the therapeutic agent is lipophilic or hydrophilic.

In some embodiments, the infusion solution is administered intrathecally, for example:
at a lateral ventricle, third ventricle, fourth ventricle, cisterna magna, or lumbar subarachnoid space of the subject;
   proximally to a choroid plexus of the subject;
or a combination of any of the foregoing.

In some embodiments, the infusion solution is administered to the subject (e.g., a murine subject) at a rate of up to about 20 μL over up to about 20-80 seconds.

In some embodiments, the infusion solution is administered (e.g., a murine subject) at a rate of about 10 μL over about 28-33 seconds.

In some embodiments, the administering the infusion solution is performed with a 30 gauge needle or a 33 gauge needle.

In some embodiments, the infusion solution has a volume of up to about half of a volume of a CSF of the subject (e.g., a human subject).

In some embodiments, the infusion solution has a volume of about one-fifth to about one half of a volume of a CSF of the subject (e.g., a human subject).

In some embodiments, the subject has, is suspected to have, or is at risk for having a stroke, neurodegenerative disease, neuroinflammation, cancer affecting a central nervous system (CNS), disease or infection of the CNS, cerebrovascular disease, or a combination thereof.

In some embodiments, the subject has or is suspected to have a traumatic brain injury (TBI), traumatic spinal cord injury, or both.

In some embodiments, the TBI comprises a recurrent TBI (rTBI), single TBI (sTBI), or both.

In some embodiments, the neurodegenerative disease comprises or is associated with amyotrophic lateral sclerosis (ALS), frontotemporal dementia (FTD), multiple sclerosis (MS), Alzheimer's disease and related dementias (ADRD), Guillain-Barre syndrome, hexanucleotide repeat expansion (HRE) in a C9orf72 gene, C9orf72 haploinsufficiency, cytoplasmic accumulation of TAR DNA-binding protein 43 kDa (TDP-43), or a combination thereof.

In some embodiments, the cancer is primary, metastatic, associated with leptomeningeal metastases (LM), or a combination thereof.

In some embodiments, the cancer comprises brain cancer.

In some embodiments, the cancer comprises medulloblastoma (MB).

In some embodiments, the disease of the CNS is a lysosomal storage disorder.

In some embodiments, the subject has, is suspected to have, or is at risk for having an abnormal deposit comprising a neurotoxin, an inflammatory cytokine, or both.

In some embodiments, the neurotoxin is a small molecule or a protein.

In some embodiments, the abnormal deposit comprises amyloid-β, Tau protein, α-synuclein, or a combination thereof.

In some embodiments, the subject is a pediatric subject.

In some embodiments, the subject is an adult subject.

In another aspect, the disclosure herein provides an infusion solution for use in enhancing delivery of a therapeutic agent to a parenchyma or perivascular space of a subject in need thereof, comprising a hypertonic solution and a therapeutic agent.

In another aspect, the disclosure herein provides an infusion solution for use in treating a subject in need thereof, comprising a hypertonic solution.

In another aspect, the disclosure herein provides an infusion solution for use in enhancing cerebrospinal fluid flow in a subject in need thereof, comprising a hypertonic solution.

In another aspect, the disclosure herein provides an infusion solution for use in reducing an abnormal protein deposit in a subject in need thereof, comprising a hypertonic solution.

In another aspect, the disclosure herein provides an infusion solution for use in stimulating production of cerebrospinal fluid (CSF) in a subject in need thereof, comprising a hypertonic solution.

In some embodiments, the infusion solution further comprises a therapeutic agent.

In another aspect, the disclosure herein provides a composition for use in manufacture of a medicament for enhancing delivery of a therapeutic agent to a parenchyma or perivascular space of a subject in need thereof, comprising an infusion solution, wherein the infusion solution comprises a hypertonic solution and a therapeutic agent.

In another aspect, the disclosure herein provides a composition for use in manufacture of a medicament for treating a subject in need thereof, comprising an infusion solution, wherein the infusion solution comprises a hypertonic solution and a therapeutic agent.

In another aspect, the disclosure herein provides a composition for use in manufacture of a medicament for enhancing cerebrospinal fluid flow in a subject in need thereof, comprising an infusion solution, wherein the infusion solution comprises a hypertonic solution and a therapeutic agent.

In another aspect, the disclosure herein provides a composition for use in manufacture of a medicament for reducing an abnormal protein deposit in a subject in need thereof, comprising an infusion solution, wherein the infusion solution comprises a hypertonic solution and a therapeutic agent.

In another aspect, the disclosure herein provides a composition for use in manufacture of a medicament for stimulating production of cerebrospinal fluid (CSF) in a subject in need thereof, comprising an infusion solution, wherein the infusion solution comprises a hypertonic solution and a therapeutic agent.

In some embodiments, the composition further comprises at least two therapeutic agents.

In some embodiments, the hypertonic solution comprises: a total osmolarity of about 1.1 times (1.1×) to about 8 times (8×) a total osmolarity of the CSF of the subject, an osmolarity of Na⁺ about 1.1 times (1.1×) to about 8 times (8×) an osmolarity of Na⁺ in the CSF of the subject, an osmolarity of K⁺ about 1.1 times (1.1×) to about 8 times (8×) an osmolarity of K⁺ in the CSF of the subject, an osmolarity of Ca²⁺ about 1.1 times (1.1×) to about 8 times (8×) an osmolarity of Ca²⁺ in the CSF of the subject, an osmolarity of Mg²⁺ about 1.1 times (1.1×) to about 8 times (8×) an osmolarity of Mg²⁺ in the CSF of the subject, an osmolarity of Cl⁻ about 1.1 times (1.1×) to about 8 times (8×) an osmolarity of Cl⁻ in the CSF of the subject, an osmolarity of HCO₃⁻ about 1.1 times (1.1×) to about 8 times (8×) an osmolarity of HCO₃⁻ in the CSF of the subject, or any combination of the foregoing.

In another aspect, the disclosure herein provides a kit comprising an infusion solution or a composition and a composition comprising an additional therapeutic agent and/or a device for administration of the solution and/or composition.

In another aspect, the disclosure herein provides a kit comprising a lyophilized form of an infusion solution or a composition and a composition comprising an additional therapeutic agent and/or a device for administration of the solution and/or composition.

In another aspect, the disclosure herein provides a kit comprising a hypertonic solution, wherein the hypertonic solution comprises: a total osmolarity of about 1.1 times (1.1×) to about 8 times (8×) a total osmolarity of the CSF of the subject, an osmolarity of Na⁺ about 1.1 times (1.1×) to about 8 times (8×) an osmolarity of Na⁺ in the CSF of the subject, an osmolarity of K⁺ about 1.1 times (1.1×) to about 8 times (8×) an osmolarity of K⁺ in the CSF of the subject, an osmolarity of Ca²⁺ about 1.1 times (1.1×) to about 8 times (8×) an osmolarity of Ca² in the CSF of the subject, an osmolarity of Mg²⁺ about 1.1 times (1.1×) to about 8 times (8×) an osmolarity of Mg²⁺ in the CSF of the subject, an osmolarity of Cl⁻ about 1.1 times (1.1×) to about 8 times (8×) an osmolarity of Cl in the CSF of the subject, an osmolarity of HCO₃⁻ about 1.1 times (1.1×) to about 8 times (8×) an osmolarity of HCO₃⁻ in the CSF of the subject, or any combination of the foregoing.

In another aspect, the disclosure herein provides a kit comprising a lyophilized hypertonic solution, wherein the hypertonic solution comprises: a total osmolarity of about 1.1 times (1.1×) to about 8 times (8×) a total osmolarity of the CSF of the subject, an osmolarity of Na⁺ about 1.1 times (1.1×) to about 8 times (8×) an osmolarity of Na⁺ in the CSF of the subject, an osmolarity of K⁺ about 1.1 times (1.1×) to about 8 times (8×) an osmolarity of K⁺ in the CSF of the subject, an osmolarity of Ca²⁺ about 1.1 times (1.1×) to about 8 times (8×) an osmolarity of Ca²⁺ in the CSF of the subject, an osmolarity of Mg²⁺ about 1.1 times (1.1×) to about 8 times (8×) an osmolarity of Mg²⁺ in the CSF of the subject, an osmolarity of Cl⁻ about 1.1 times (1.1×) to about 8 times (8×) an osmolarity of Cl in the CSF of the subject, an osmolarity of HCO₃ about 1.1 times (1.1×) to about 8 times (8×) an osmolarity of HCO₃⁻ in the CSF of the subject, or any combination of the foregoing.

### Example Models of Behavioral Consequences of Traumatic Brain Injury.

Using behavioral testing as well as structural and functional MRI, mild acute brain injury (e.g., injury that causes no overt brain damage as detected by standard structural MRI and histological assessment) and how it affects brain function and behavior was studied. These studies were among the first in the fields of strokes and TBI conducted in small animals and demonstrated that even so-called “mild” injuries can have a profound impact on the brain's functional integrity. There is evidence that mild, concussive closed head TBI triggers cortical spreading depolarizations (CSD), and that CSDs are associated with worse histopathology and functional deficit recovery after mild TBI. See, for example, references (32) and (36).

### Examples of Acute Brain Injury

Cerebral small vessel disease (CSVD) is highly prevalent among the elderly and represents a vascular contribution to cognitive impairment and dementia (VCID). The specific contribution CSVD to functional outcome after acute brain injury (TBI and ischemic stroke) is incompletely understood. Understanding this issue is important considering the high prevalence of CSVD among patients with vascular risk factors and cognitive impairment. Clinical investigations have highlighted that worse CSVD is associated with greater functional deficits, reduced functional deficit recovery, and an overall increased risk for functional dependence. These results highlight that CSVD may serve as a marker of the brain's intrinsic capacity to withstand acute injury. Accordingly, CSVD may serve as an important therapeutic target to improve outcome after acute brain injury including TBI and stroke. See, for example, reference (41).

### Examples of Central Nervous System Drug Delivery

Systemically circulating molecules face a multitude of barriers to effective CNS delivery, including rapid clearance and inadequate transport across the blood brain barrier (BBB). Encapsulation of active agents within polymeric nanoparticles provides an opportunity to enhance drug tolerability and delivery to the CNS. We have developed approaches to enhancing drug transport across the BBB in glioblastoma (48, 49, 93, 96) and amyotrophic lateral sclerosis (47). More recently, we have focused on the intrathecal route (including lumbar, ventricular, or cisterna magna infusion) for achieving CNS delivery of drugs and drug loaded nanoparticles. Our group has made significant contributions to this field, including the first-ever comprehensive description of nanoparticle fate following intrathecal injection in mice (4). We recently published a major review on this topic, summarizing current knowledge of the intrathecal route for drug delivery and imaging (5).

### Examples of Imaging Nanoparticle Fate

One of the major challenges to the development of targeted nanomedicine is the difficulty of tracking the movement of polymeric carriers within the body. New approaches for imaging nanoparticle delivery to and clearance from the brain parenchyma (45) have been developed. The fundamentally distinct fates of carrier vs. payload in the brain, CNS regional distribution, and targeting efficacy in treatment of glioblastoma have been studied (49). These foundational observations highlighted major methodological challenges in the field and contributed to development of a new approach for barcoding polymeric nanoparticles with bright, multi-spectral quantum dots (QD), which enabled evaluation of targeted drug delivery utilizing within-subject controls (93). Barrier breakdown influence on nanoparticle deposition in intracranial glioma (50) and following surgical resection have been studied. Collectively, these studies reinforce the significance of imaging nanoparticle movement toward design of novel therapeutics targeting the nervous system.

Additional PET and MALDI techniques are currently in development for multimodal imaging of nanoparticle fate in leptomeningeal metastasis and to deepen understanding of the role of surgical tissue injury in mediating CNS deposition of systemically circulating nanoparticles.

### Examples of Therapeutic Development

Nanoparticle imaging and tracking led to nanomedicine scale-up in a porcine model (46). Therapeutic development in nonhuman primate models (53) and translation of new intrathecal drug delivery approaches in humans (52) have been studied. Preclinical dose determination work enabled new clinical trials at Children's Memorial Hospital in Houston, TX (94,95).

### Examples of Tissue Engineering in the Central Nervous System

Several approaches for engineering 3D, brain mimetic microenvironments have been developed. These materials include a hyaluronic acid-gelatin composite that is uniquely stable in aqueous environments (96), as well as “smart polymer” scaffolds that form physical gels at body temperature but dissolve upon cooling to room temperature, which enables capture and release of encapsulated cells (97,98) and electrospun polyester scaffolds that mimic the unique structure of the subarachnoid space. These materials have been shown to enable study of glioblastoma invasion in response to environment stimuli and serial passage and facile recollection of cells (97). Conditions under which cancer stem cells can be enriched from patient derived glioblastoma have been demonstrated; these cells are shown to be self-renewing, multipotent, and radiation resistant in conditions that mimic the perivascular niche (98). These efforts have recently been extended to study metastatic medulloblastoma (86).

### Examples of Nanoparticle Engineering

In addition to the novel fabrication work described in the sections above, a variety of nanoparticles with size, loading, and release characteristics optimized for specific biological applications have been engineered. A foundational methods article provides comprehensive description of formation of polyester nanoparticles by emulsion-solvent evaporation (99). New approaches that manipulate the encapsulation within and release of drugs from polymeric nanoparticles have been developed, including novel fabrication strategies and manipulation of emulsion conditions for multi-layer systems (100), particle size fractionation for protein delivery (101), and novel self-assembly approaches (102).

### Exemplification

Manipulation of CSF production and egress for treatment of conditions affecting the CNS (e.g., for drug delivery) will be transformative to the ability to safely and selectively treat the CNS with IT-administered nanomedicine. Manipulation of CSF production and egress for the purpose of drug delivery represents major conceptual innovation, enabling researchers and healthcare practitioners to safely manipulate an endogenous physiological system for improved treatment efficacy. The studies disclosed herein were and will be both conceptually and methodologically innovative and will improve foundational understanding of how to best treat CNS conditions as well as promote progress toward new therapies to be tested that hold substantial potential to improve human health.

The tables below summarize various CSFs, solutions, and nanoparticles, including those referenced and/or used in the Examples.

TABLE 1
Composition* of physiological CSF.

| | Human | Mouse | Rat |
|---|---|---|---|
| Approximate Total (mOsm) | 280 | 290 | 330 |
| Na⁺ (mOsm) | 140-145 | 100-200 | |
| K⁺ (mOsm) | 2.86 | 3.1 | |
| Ca²⁺ (mOsm) | 1.14 | 1.2 | |
| Mg²⁺ (mOsm) | 1.1 | 0.9 | |
| Cl⁻ (mOsm) | 113 | 126 | |
| HCO₃⁻(mOsm) | 23.3 | N/A | |
| pH | 7.3 | N/A | |

*The exact composition of CSF is known to vary by a number of experimental and physiological factors, including variables such as sampling location (e.g., ventricles vs lumbar subarachnoid space), sex (male vs female), age, disease, etc. The values listed are typical levels but do not encompass all experimental and physiological conditions.

TABLE 2
Composition of 1x aCSF as reported/used in the field and
compositions of 1x aCSF and 1x PBS used in Examples 1-7.

| | Mouse 1x aCSF | Mouse 1x* aCSF | |
|---|---|---|---|
| | (in field) | (in Examples) | 1x PBS |
| Total (mOsm) | 300-360 | 310 | 330 |
| Na (mOsm) | 120-170 | 125 | 157 |
| K (mOsm) | 2-4 | 3 | 4.5 |
| Ca (mOsm) | 1-2 | 2 | 1 |
| Mg (mOsm) | 0-3 | 1 | 0.5 |
| Cl (mOsm) | 100-200 | 134 | 140 |
| HCO3 (mOsm) | 10-30 | 26 | N/A |
| pH | 6-9 | variable | 7.4 |

*Concentrations of aCSF constituents indicated in Table 2 are scaled up as numerically indicated in 2x aCSF (e.g., 620 mOsm total), 4x aCSF, 6x aCSF, and 8x aCSF.

TABLE 3
Composition of infusion solutions used in Examples 1-7 and 11.

| FIGS. | Control/Isotonic | Hypertonic | Tracer | Nanoparticles | Other |
|---|---|---|---|---|---|
| 1 | 1x PBS | 5x PBS | ¹⁸F | N/A | |
| 3 | | | | Gold, spherical (10 | |
| | | | | nm, 100 nm) or rod | |
| | | | | (15 × 50 nm) | |
| 4 | | | | Polystyrene, stretched | |
| | | | | or spherical | |
| 5 | 1x aCSF | 2x, 8x | Fl | 100 nm polystyrene | |
| | | aCSF | | | |
| 6 | 1x aCSF | 2x aCSF | Fl | 35-40 nm nucleic | |
| | | | | acid | |
| 7 | 1x aCSF | 2x aCSF | Fl | N/A | Rhodamine B |
| 8 | 1x aCSF | 2x aCSF | Fl | 20, 40, and 100 nm | Hydrazide |
| | | | | polystyrene | |
| 9 | 1x aCSF | 2x aCSF | Fl | 50 nm polyester | |
| 10 | 1x aCSF | 2x aCSF | Fl | 20, 40, and 100 nm | Rhodamine B, |
| | | | | polystyrene | Hydrazide |
| 11 | 1x aCSF | 2x aCSF | Fl | 20, 40, and 100 nm | |
| | | | | polystyrene | |
| 13 | 1x aCSF | 2x aCSF | Fl | 40 and 100 nm | |
| | | | | polystyrene | |
| 14 | 1x aCSF | 2x aCSF | Fl | 100 nm polystyrene | Hydrazide |
| 20 | 1x aCSF | 2x aCSF | | N/A | |
| 21, 22 | 1x aCSF | 2x aCSF | Fl | 20, 40, and 100 nm | |
| | | | | polystyrene | |
| 23-25 | 1x aCSF | 2x, 4x | Fl | 100 nm polystyrene | |
| | | aCSF | | | |
| 26 | 1x aCSF | 2x, 4x, 6x, | Fl | 100 nm polystyrene | |
| | | 8x aCSF | | | |
| 27 | 1x aCSF | 2x aCSF | Fl | 100 nm polystyrene | |
| 28 | 1x aCSF | 2x aCSF | F1 | 40 nm nucleic acid | |
| 29 | 1x aCSF | 2x aCSF | Fl | N/A | ALEXA FLUOR ® |
| | | | | | Hydrazide |
| 30 | 1x aCSF | N/A | Fl | Non-PEGylated and | |
| | | | | PEGylated | |
| | | | | polystyrene | |

Fl. = fluorescent tag, added to all nanoparticle systems

TABLE 4
Properties of Small Molecules and Nanoparticles.

| Substance | Properties |
|---|---|
| CDN₅ | beta-cyclodextrin-poly(beta-amino ester) |
| | nanoparticles; deformable, sometimes unstable |
| | and biodegradable spheres |
| Gold NPs | Rigid, nondegradable spheres |
| Hydrazide A594 | Small, water soluble |
| Rhodamine | Small, water soluble, mildly lipophilic |
| Polystyrene NPs | Rigid, nondegradable spheres |
| (PSNP 20 nm) | |
| Polystyrene NPs | Rigid, nondegradable spheres |
| (PSNP 40 nm) | |
| Polystyrene NPs | Rigid, nondegradable spheres |
| (PSNP 100 nm) | |
| Polyester NPs | Deformable, sometimes unstable and |
| | biodegradable spheres |
| Nucleic acid NPs | Biodegradable with varying surface properties |
| (NANPs) | |

### Example 1

### Cerebrospinal Fluid (CSF) Flow Enhancement (CFE)

### Materials and Methods

A 26 G guide cannula was inserted into the right lateral ventricle of healthy Sprague Dawley rats under isoflurane anesthesia. F-18 Fluoride ion (¹⁸F, radiofluorine) was diluted in buffer to achieve an isotonic (1× phosphate-buffered saline (PBS), 330 mOsm) or hypertonic (5× PBS, 1650 mOsm) composition. Infusions were conducted at a rate of 1 μL/min to deliver a total volume of 10 μL. The cannula was left in place for 5 minutes to reduce backflow. Data were collected on a Siemens MicroPET Focus 300 during and after the infusion and reconstructed into 15 minute frames. Co-infusion of fluorescent dye confirmed injection location. Negative controls included infusion of ¹⁸F into the parenchyma and infusion of F-18 fluorodeoxyglucose (¹⁸F-FDG) into the ventricles. Both negative controls yielded no movement of tracer (data not shown).

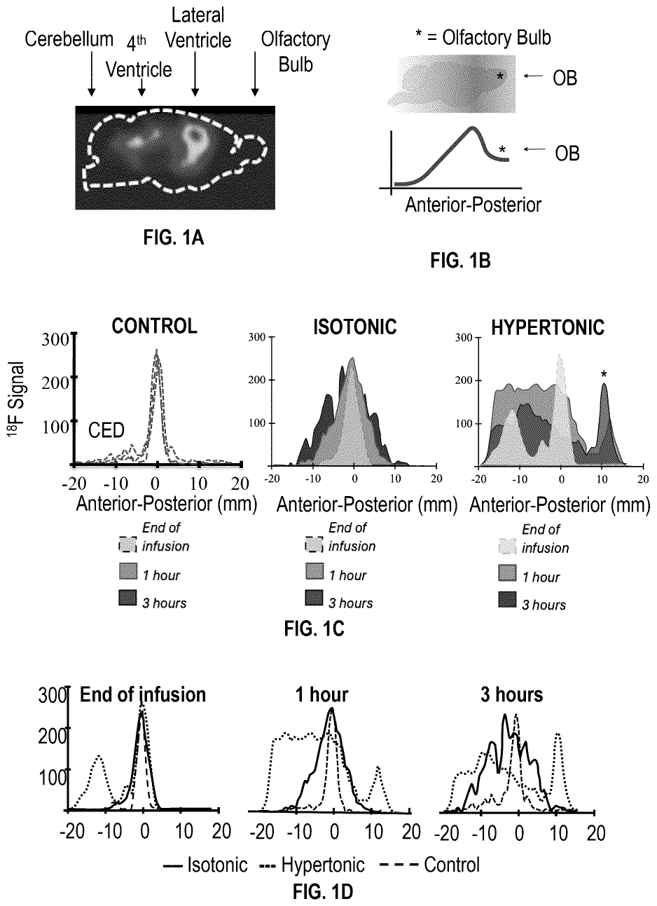

Tracer distribution was quantified along the anterior-posterior axis of the brain (FIG. 1B). The fluoride ion is small (18 Da) and water soluble, so it directly tracks CSF (FIG. 1A). These data demonstrate significant enhancements in CSF movement through the parenchyma and distinct spatial patterns of accumulation or clearance in hypertonic conditions.

### Results

Ventricles were better defined and tracer distribution was more widespread in the hypertonic condition, suggestive of increased CSF flow (i.e., CFE) through the ventricles and through the parenchyma for the hypertonic condition. At 1 hour post-infusion, more widespread distribution for the hypertonic condition was observed. The entire brain was visible. Olfactory bulb was observable, which was never seen in other conditions. These data are suggestive of increased CSF flow throughout the brain parenchyma and redirection of normal CSF flow for the hypertonic condition. At 3 hours post-infusion, more widespread distribution of tracer and more prominent olfactory bulb signal in the hypertonic condition was observed, suggestive of increased fluid clearance from the brain in the hypertonic condition.

Together, these data demonstrated extensive movement of fluid throughout the brain and movement into the spinal cord and olfactory bulb in the hypertonic condition (FIGS. 1C, 1D). Infusion of hypertonic solution into the lateral ventricle dramatically enhanced fluid distribution in the rat CNS.

### Example 2

### Nanoparticle-Enhanced Drug Delivery to the CNS

### Materials and Methods

### Polymer Synthesis and Nanoparticle Formulation.

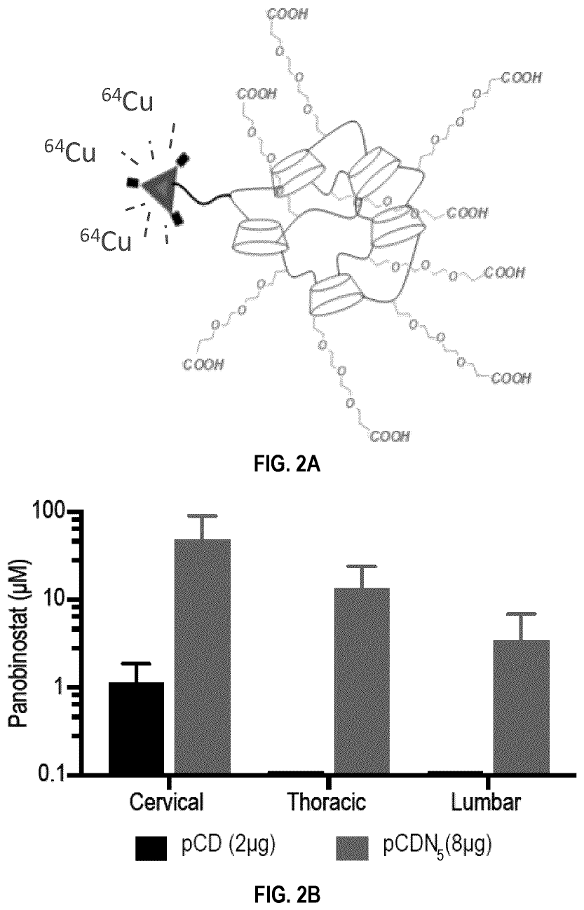

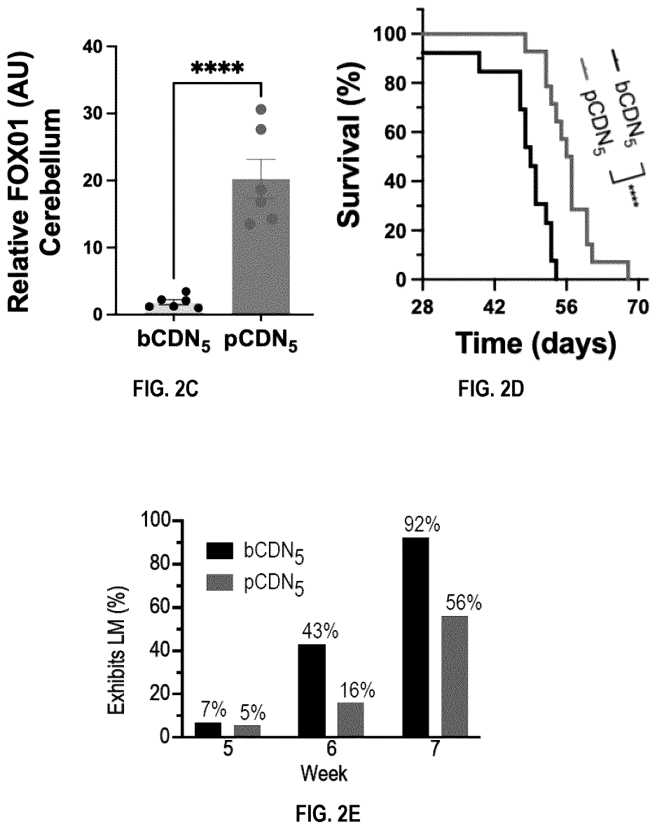

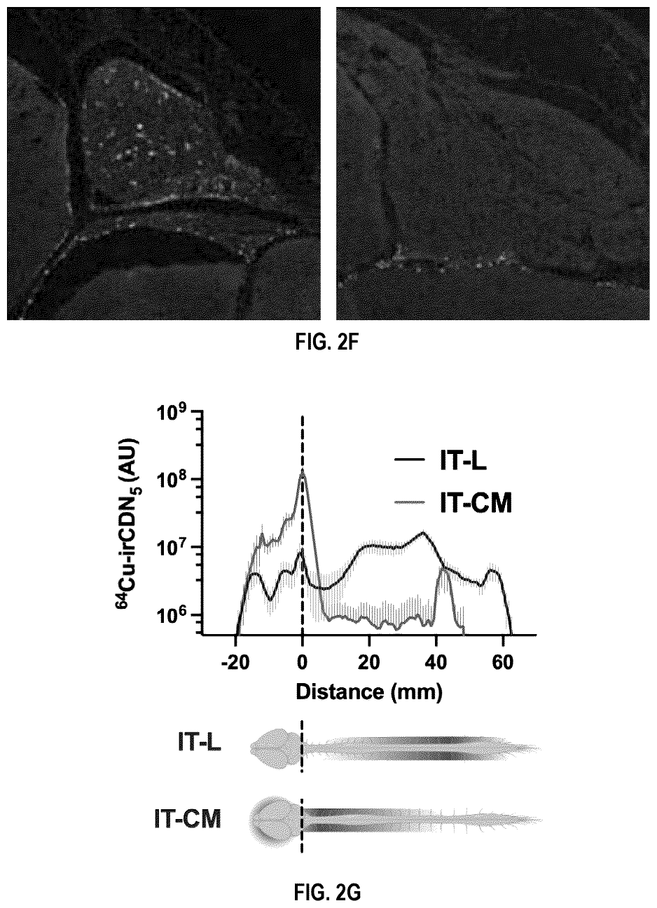

CDN-5 polymer (FIG. 2A) was synthesized by adapting on previously described methods (102), the contents of which are incorporated herein in their entirety. Acrylated beta-cyclodextrin (acr-CD) was produced by dissolving beta-cyclodextrin (CD, 2 mmol) in N-Methyl-2-pyrrolidone (NMP) and reacting with acrolyl chloride (24.7 mmol). This solution was first mixed under ice-cold conditions for 1 hour, after which it was heated to 21° C. and allowed to react for 48 hours. The resultant acr-CD was precipitated in distilled water, collected, washed by filtration, and lyophilized for 48 hours. To generate CDN-5 polymer, acr-CD (0.03 mmol) was mixed with 1,6-hexanediol diacrylated (0.65 mmol) and bi-functional poly(ethylene glycol) (COOH-PEG-amine, 0.95 mmol). These solutions were heated to 65° C., allowed to react while stirring at 600 revolutions per minute (rpm) for 12 hours, and dried under vacuum. The resultant crude was dispersed into a large volume of distilled water (50 mL), passed through a 0.45 μm bottle-top filter to remove large aggregates, and thoroughly washed via ultrafiltration (AMICON® Ultra-15 centrifugal filers, 10 kilodalton (kDa) molecular weight cutoff (MWCO), 15 mL capacity) for two 20 minute spins at 5000 relative centrifugal force (RCF). CD and CDN formulations loaded with Panobinostat (CAS No. 404950-80-7; PubChem CID 6918837) were generated by doping hydrophobic payload into an aqueous dispersion of polymer. Nanoparticle payloads for this work comprised either Panobinostat (20 mg, pCDN₅) or MIGLYOL® 812N, a triglyceride ester of saturated caprylic and capric fatty acids used as a drug carrier (0.25 mg, bCDN₅), in 50 μL of dimethyl sulfoxide (DMSO). Aqueous dispersions of payload with polymer were maintained on a rocker at room temperature for 18 hours, during which time hydrophobic payloads enter the polymer network to facilitate self-assembly. This is a formulation approach that optimized in previous work (102). The resultant nanoparticles were concentrated via ultrafiltration (AMICON® Ultra-15 filters, 3 kDa MWCO, 0.5 mL capacity) for four 20 minute spins at 5000 RCF, lyophilized, and stored at −80° C. until use.

### Radiochemistry

NODA-GA grafted CDN polymers were obtained via a modified CDN synthetic protocol, employing amino-NODA-GA chelator (0.5 wt % of amino-PEG₃-COOH) in the Michael addition reaction. NODA-GA grafted polymers were formed into nanoparticles through the addition of hydrophobic payload, as described above, added to sodium acetate buffer (pH=6.5), and treated with radioactive ⁶⁴CuCl₂ for chelation. This mixture was incubated for 1 hour at 30° C., after which radiochemical purity was assessed via thin layer chromatography (TLC) analysis of the reaction mixture with scintillation counting. The reaction mixture was further concentrated via ultrafiltration to enrich the radiochemical purity as needed for in vivo experiments. Colloidal stability and ⁶⁴Cu retention studies of nanoparticles were carried out in PBS or artificial cerebrospinal fluid (aCSF) with or without 10% fetal bovine serum (FBS) (diluted in water).

### Tumor Inductions

Med-411FH PDX tumor cells engineered to express luciferase were stereotaxically implanted into the cerebellum of 8-10 week old NOD scid gamma (NSG) mice. Mice were anesthetized with 2% Isoflurane (2 L/min, MWI Veterinary Supply #NDC 66794-013-25). An incision was carefully made in the skin over the posterior skull and a hole in the skull was created with a 16 gauge needle 2 mm posterior to lambda at the midline. 1×10⁵ cells suspended in 5 μL NEUROCULT® media (STEMCELL Technologies, Vancouver, Canada; #5750) were injected using a blunt-end Hamilton syringe (The Hamilton Company, Reno, NV, USA; #88000) angled at 350 and targeting 3.5 mm deep into the cerebellum. Tumor-bearing mice were imaged 1×/week using an In Vivo Imaging System (IVIS) Spectrum system (Perkin-Elmer, Waltham, MA, USA) under anesthesia after intraperitoneal luciferin injection (150 mg/kg). All mice were monitored for humane endpoint morbidity or toxicity (loss of balance and coordination, >20% weight loss, inactivity), whereupon they were euthanized.

### Substance Administration.

Formulations were resuspended in 1× aCSF and administered to anesthetized mice via direct infusion into the cisterna magna (10 μL total volume, administered over 30-60 seconds). Following infusion, the needle was immediately withdrawn, and subjects were placed in a clean cage and observed until ambulatory.

### Pharmacodynamic Measurements

Cerebellum and spinal cord tumors from acutely CDN-treated Med-411FH tumor-bearing mice were dissected and enzymatically dissociated into a single cell suspension. For Quantitative Reverse Transcriptase PCR (qRT-PCR), total RNA was extracted from cerebellum or spinal cord tumor cells using the RNEASY® Plus Micro Kit for RNA purification (Qiagen, Germantown, MD, USA; #74034). cDNA was generated using iScript Reverse Transcription Supermix (BIO-RAD, Hercules, CA, USA; #1708840). RT-PCR was performed using iQ SYBR® Green Supermix (BIO-RAD #1708880) with a CFX96 Real Time PCR System (BIO-RAD). The following primers were used: FOXO1 Forward: 5′-TGATAACTGGAGTACATTTCGCC-3′ (SEQ ID NO: 1), FOXO1 Reverse: 5′-CGGTCATAATGGGTGAGAGTCT-3′ (SEQ ID NO: 2), BETA-ACTIN Forward: 5′-TGACGTGGACATCCGCAAAG-3′ (SEQ ID NO: 3), BETA-ACTIN Reverse: 5′-CTGGAAGGTGGACAGCGAGG-3′ (SEQ ID NO: 4).

### Efficacy

Med-411FH tumor-bearing mice were imaged by IVIS 9 days after cell transplantation and allocated into treatment groups to balance tumor sizes at initiation of treatments. Intrathecal cisterna magna administration (IT-CM) treatments began on day 12 after cell implantation, and comprised 6 g pCDN₅ (nanoparticle repeat-dose maximum tolerated dose (MTD)) or equivalent CDN₅ mass. Subsequent IT-CM treatments were performed 2×/week through to humane endpoint. Endpoint was defined as greater than 20% weight loss or the appearance of neurological symptoms.

### Imaging

Positron Emission Tomography (PET)/Computed Tomography (CT) imaging studies were performed with a Siemens Inveon PET/CT multimodal system for small animals (Siemens Medical Solutions, Knoxville, TN). Scans were performed in Inveon Aquisition Workplace software with the following workflow: 1) PET emission acquisition, 2) CT acquisition, and 3) PET reconstruction. CT acquisition data were collected with the following parameters: x-ray maximum voltage of 80 kV, maximum anode current of 500 μA, exposure time of 260 msec for each of 120 rotation steps over a total rotation of 220° C. PET/CT reconstructions were conducted with a 2D Ordered Subset Expectation Maximization (OSEM2D) and a Feldkamp cone-beam algorithm, using a Shepp-Logan filter. PET and CT image fusion and image analysis were performed using ASIPro, Inveon Research Workplace (Siemens Preclinical Solutions) and AMIRA (version 3.1). PET/CT images are displayed as overlaid maximum intensity projections (MIPs).

### Drug Distribution

Drug distribution was assessed by matrix-assisted laser desorption ionization mass spectrometry imaging (MALDI-MSI). Mice received an infusion of 8 μg pCDN5 or 2 μg pCD, representing the single-dose maximum tolerated dose (MTD) for each formulation. Two hours after treatment, subjects were euthanized and the brains and spinal cords were extracted. Tissues were snap-frozen in liquid nitrogen, mounted onto a specimen chuck, and sectioned coronally at 10 μm thickness (Microm HM550, Thermo Scientific, Waltham, MA, USA). The tissue sections were thaw-mounted onto indium tin oxide (ITO) slides. A tissue microarray (TMA) mold was used to prepare a collagen mimetic model for panobinostat quantitation by MALDI-MSI. Panobinostat concentrations ranging from 0.1-50 μM were spiked into rat tail collagen and pipetted into 1.5 mm core diameter channels of the 40% gelatin TMA mold and frozen at −80° C. The collagen mimetic mold was sectioned, and thaw mounted on top of control mouse brain homogenate, adjacent to the mouse brain tissue sections for analysis. Twelve serial sections moving medial to lateral were taken every 150 m and thaw-mounted to Bruker Big Slides (Bruker Scientific LLC, USA Part No. 8259387), with serial sections preserved for hematoxylin and eosin (H&E) staining. The ITO slide mounted with the tissue and mimetic mold sections were placed in a vacuum desiccator before matrix deposition. 2,5-dihydroxybenzoic acid (160 mg/mL) matrix was dissolved in 70:30 methanol: 0.1% trifluoroacetic acid (TFA) with 1% DMSO and applied using a TM sprayer (HTX Technologies, Chapel Hill, NC, USA) at a flow rate (0.18 mL/min), spray nozzle velocity (1200 mm/min), nitrogen gas pressure (10 psi), spray nozzle temperature (75° C.), and track spacing (2 mm) for two passes. Optical microscopy images of the H&E-stained serial tissue sections were acquired using a 10× objective (Zeiss Observer Z.1, Oberkochen, Germany). A multiple reaction monitoring (MRM) approach was used for quantitative imaging of panobinostat by monitoring the transition of the precursor ion fragment to product ion (350.18-158.09) using a dual source, timsTOF fleX mass spectrometer (Bruker Scientific LLC, Billerica, MA, USA) in positive ion mode and with acquisition between m/z 100-650. Tandem MS parameters were set for a collision energy of 23 eV with an isolation width of 3 m/z. MALDI MS images were acquired with a laser repetition set to 5,000 Hz with 2,000 laser shots per 100 μm pixel. SCiLS Lab software (version 2024a premium, Bruker Scientific LLC, Billerica, MA, USA) was used for data visualization without data normalization. The average ion intensity for each spiked TMA sectioned core area was plotted against corresponding panobinostat concentration from 0.1-50 μM for calibration of the MALDI MS signal, resulting in a limit of detection (LOD) of 0.2 μM (S/N ratio of >3), and a limit of quantification (LOQ) of 0.7 μM (S/N ratio of >10).

### Results

Panobinostat loaded nanoparticles (pCDN₅) or Panobinostat solubilized in commercially purchased beta-cyclodextrin (pCD) were administered via cisterna magna infusion into healthy mice (8 or 2 μg Panobinostat dose, respectively). Drug levels were evaluated by MALDI-MSI and quantified for spinal cord regions of interest, including cervical, thoracic, and lumbar spinal cord (FIG. 2B).

Drug-empty nanoparticles (bCDN₅) or drug loaded nanoparticles (pCDN₅, 8 μg panobinostat) were administered to NSG mice bearing late-stage, patient-derived medulloblastoma exhibiting leptomeningeal metastasis. Cerebellum was extracted 6 hours after compound administration, utilizing qRT-PCR to evaluate levels of FOXO1, a pharmacodynamic marker of Panobinostat activity (FIG. 2C).

Drug-empty nanoparticles (bCDN₅) or drug loaded nanoparticles (pCDN₅, 8 μg panobinostat) were administered to NSG mice bearing late-stage, patient-derived medulloblastoma exhibiting leptomeningeal metastasis. In this survival study, endpoint criteria were >20% weight loss or appearance of neurological symptoms (FIG. 2D).

The prevalence of detectable metastatic disease in the spine was assessed by whole-body bioluminescent imaging at 5, 6, and 7 weeks after treatment (FIG. 2E).

Fluorescent polystyrene nanoparticles were administered to NSG mice bearing late-stage, patient-derived medulloblastoma exhibiting leptomeningeal metastasis. Sample images show metastatic lesions in the supracerebellar cistern (FIG. 2F). In one example, nanoparticles have infiltrated the lesion, while in the other they have not.

Healthy C57 mice received ⁶⁴Cu-radiolabeled bCDN₅ nanoparticles intrathecally, via the cisterna magna or lumbar routes. PET imaging enabled quantification of nanoparticle concentration along the spinal cord for these two routes of administration (FIG. 2G).

The results comprehensively establish that nanoparticle (NP) encapsulation improved small molecule bioavailability, tissue distribution, and treatment efficacy in orthotopic, patient-derived MB exhibiting LM (FIGS. 2B-2G). Panobinostat distribution in the spinal cord was dramatically improved by nanoparticle (NP) encapsulation (pCDN) compared to cyclodextrin-solubilized drug controls (pCD) (FIG. 2B). Panobinostat and controls were administered via intrathecal (IT) route and distribution was evaluated by matrix-assisted laser desorption ionization mass spectrometry imaging (MALDI-MSI). Compared to drug empty controls (bCDN), pCDNs activated their known pharmacodynamic target of FOXO1 (FIG. 2C), prolonged survival (FIG. 2D), and reduced incidence of leptomeningeal metastases (LM) (FIG. 2E). Movement of NPs from CSF into metastases can be further optimized as NPs achieved inconsistent access to LM (FIG. 2F) and spine distributions can be further optimized (FIG. 2G).

### Example 3

### Nanoparticle Geometry

### Methods

Gold nanoparticles (spherical and 100 nm (S100) or 10 nm (S10) in diameter, or rod-shaped and 15 nm by 40 nm in size (R15×40)) were administered to healthy C57 mice intrathecally via the cisterna magna. Whole-brain light field images were acquired with a stereoscope.

Green fluorescent protein (GFP)-expressing MDA-MB-231 breast cancer cells were cultured under standard conditions in vitro. Fluorescent polystyrene nanoparticles (100 nm, red) were embedded in a thin film of poly(vinyl alcohol), soaked in toluene, and stretched with a mechanical device to yield non-spherical nanoparticles with an average aspect ratio of 1:1.37+/−0.44 (length:width ratio, measured). Stretched nanoparticles or non-stretched nanoparticles (control) were resuspended in cell culture media applied to cells at a concentration of 50-100 μg/mL. After 2 hours, media was removed and cells were fixed for microscopy. Cells were stained with 4′,6-diamidino-2-phenylindole (DAPI), enabling identification of nuclear (DAPI, blue) and cytoplasmic (GFP, green) cell compartments.

### Results

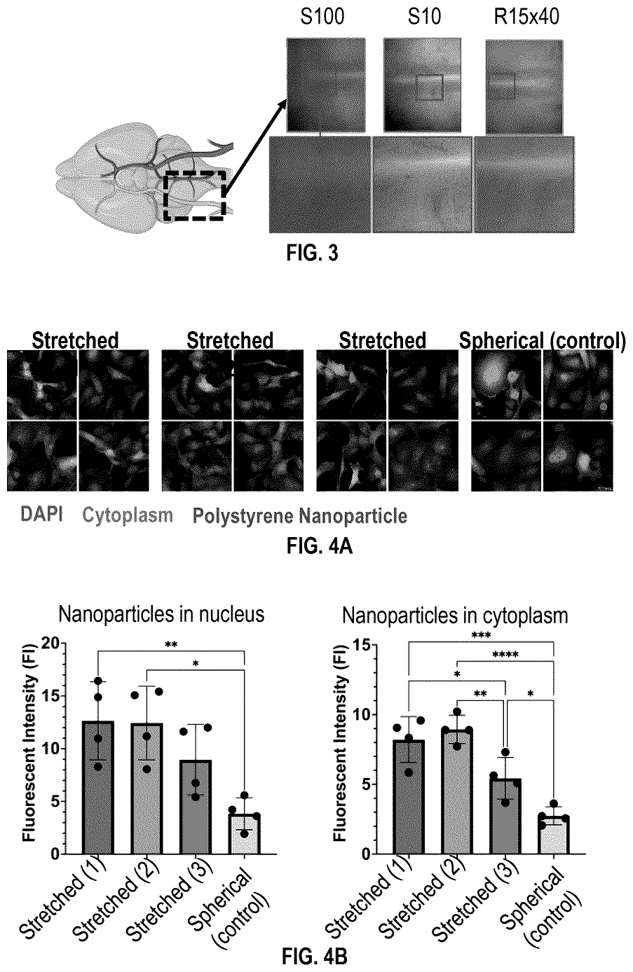

Engineering NP geometry was an example of an approach to improve distribution. Gold nanoparticle delivery to perivascular and perineuronal tissue regions on the ventral surface of the brain depended on nanoparticle geometry (FIG. 3).

Polystyrene nanoparticle (red) delivery to cell nuclei (blue) vs cytoplasm (green) depended on nanoparticle geometry in vitro (FIG. 4A). Three similar samples of stretched nanoparticles (S1, S2, S3) were compared to spherical nanoparticles (control) following 2 hours of exposure (FIG. 4B). Stretched nanoparticles outperformed spherical nanoparticles for achieving higher delivery to the cell cytoplasm as well as nucleus, suggesting that changes to shape and size can influence the ability of nanoparticles to enter cells and reach specific intracellular compartments. This may be significant for the development of nanoparticle systems that maximally benefit from co-administration with hypertonic fluids.

### Example 4

### Hypertonic aCSF and 100 nm Polystyrene NP Distribution

### Methods

10 μL isotonic (1×) and hypertonic (2×) artificial CSF (aCSF) containing fluorescently labeled, 100 nm polystyrene nanoparticles were infused into the cisterna magna of healthy C57 mice using either a 30 G or 33 G infusion needle. Two hours after substance administration, mice were euthanized and the brains were extracted, sliced into thick sections, and imaged with a fluorescent stereoscope.

### Results

Delivery of nanoparticles to deep brain regions was evident only in the hypertonic condition. Compared to the isotonic condition, nanoparticle signal was both more intense and more widespread in the hypertonic group. These 100 nm nanoparticles serve as model colloids, emphasizing the applicability of this approach in delivering therapies and flushing large substances (such as exosomes, adeno-associated viral vectors (AAV), nucleic acids, other types of nanoparticles, e.g., lipid, polymer, synthetic, biological derived, etc.). CSF flow was manipulated by infusion of mildly concentrated aCSF, which stimulated the choroid plexus to transiently generate additional CSF that propels NPs (100 nm, red) into the brain parenchyma.

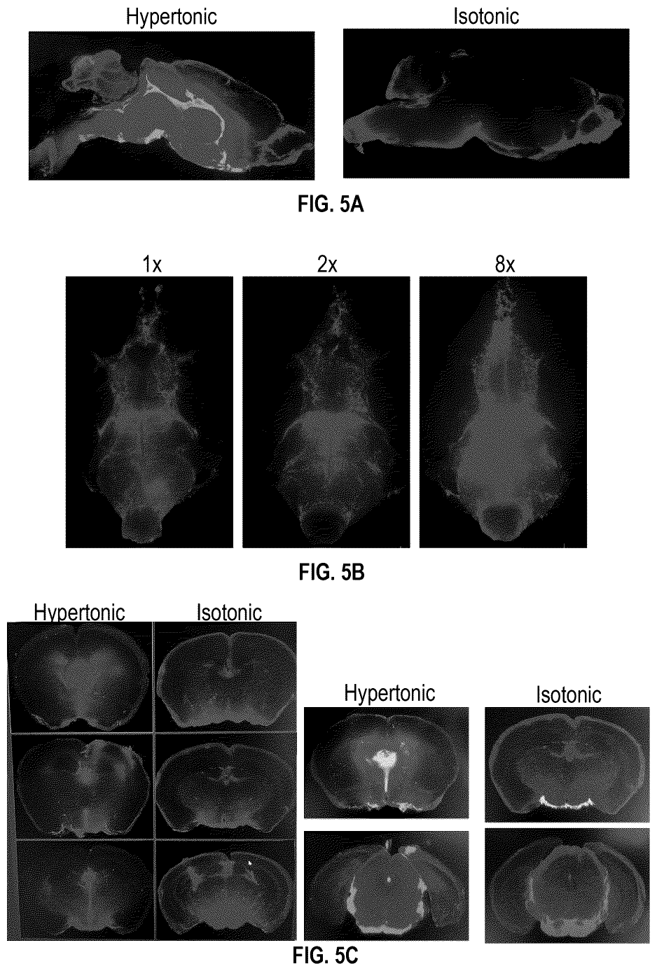

Infusion of hypertonic solution into the cisterna magna propelled large nanoparticles through the brain parenchyma in mice (FIG. 5A, 33 gauge needle used (33 G); FIG. 5B, 30G;

FIG. 5C, 33G). In the isotonic condition, these nanoparticles did not enter the parenchyma and were restricted to the subarachnoid space (SAS). In the hypertonic condition, these nanoparticles filled the ventricular system and distribute throughout the parenchyma. Nanoparticles exited through multiple routes, including cervical lymphatics, a major clearance route for CSF.

Tolerability of this approach (buffer type, volume, tonicity) was extensively optimized (data not shown). Collectively, these data demonstrate that the approach for CFE resulted in dramatic redistribution of CNS fluids, driving clearance of fluid through the olfactory bulb, spinal cord, and cervical lymphatic system. These data also provide proof of concept that even large substances are readily carried by this fluid movement to wash through the parenchyma and eventually exit the CNS.

### Example 5

### Nucleic Acid Nanoparticles (NANPs)

### Materials and Methods

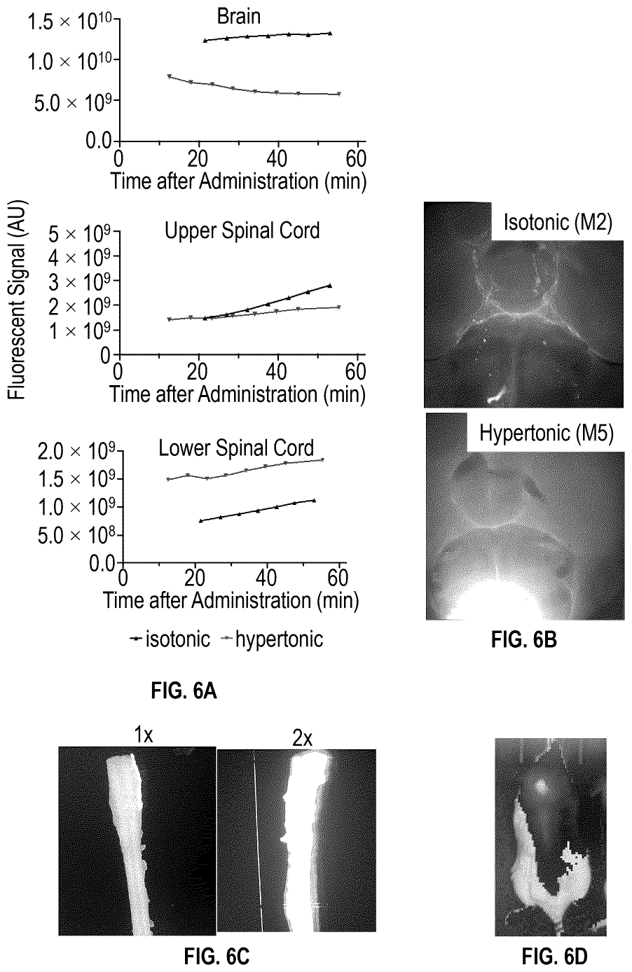

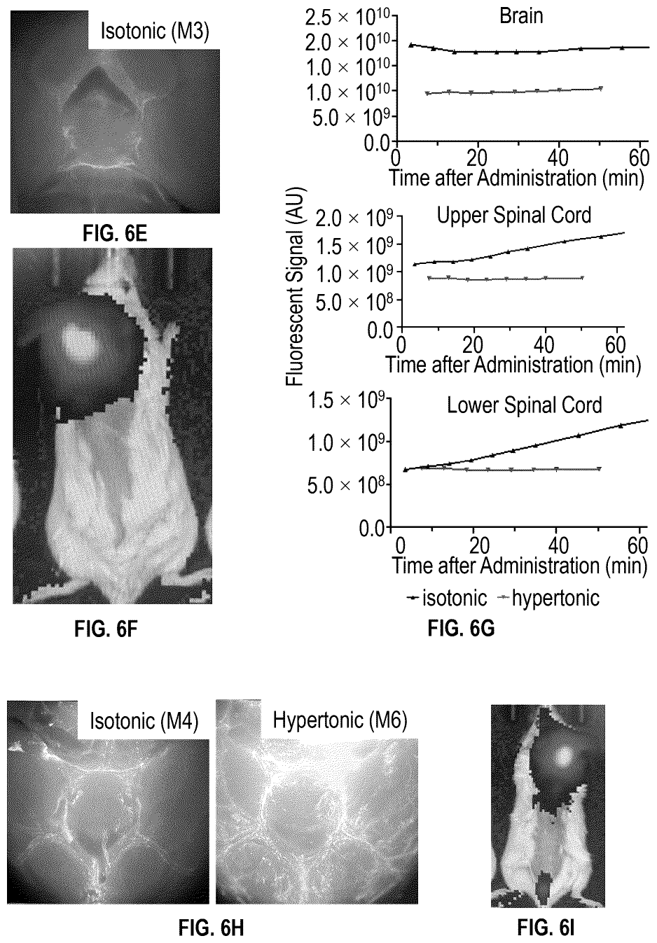

Nanoparticles composed of nucleic acids were infused as model nanoparticles (111). Three NANP formulations were used, each exhibiting distinct surface properties. NANP-A was the base nanoparticle (35-40 nm) (FIGS. 6A-6D). NANP-B was PEGylated (40-50 nm) (FIGS. 6E-6F). NANP-C was lipidated (35-40 nm) (FIGS. 6G-6I).

10 μL isotonic (1×) and hypertonic (2×) artificial CSF (aCSF) containing fluorescently labeled, NANPs were infused into the cisterna magna of healthy C57 mice. Following substance administration, mice were immediately placed in a whole-body fluorescent imaging system and maintained under isoflurane anesthesia for approximately 2 hours. Mice were imaged in regular intervals, and regions of interest (ROIs; head, lower spinal cord) were overlayed on images to enable quantification of nanoparticle distribution to different tissues of the CNS (FIGS. 6A, 6D; 6G, 6I). Fluorescence measurements are reported as arbitrary units (AU). At the termination of the experiment, brain and spinal cord were extracted from each mouse (FIGS. 6B, 6C; 6E; 6H). In FIGS. 6B, 6E, and 6H the ventral surface of the brain was imaged with a fluorescent stereoscope. In FIG. 6C, the intact spinal cord was imaged with a fluorescent stereoscope.

### Results

Co-infusion of hypertonic solution into the cisterna magna propelled large (50 nm) nanoparticles (red) from the head to the lower spinal cord ROIs (FIG. 6A). Greater nanoparticle delivery (as evidenced by a brighter fluorescent signal) was achieved on the ventral surface of the mouse brain (FIGS. 6B, 6H) and in the spinal cord (FIG. 6C) under hypertonic conditions compared to isotonic control. A whole-body fluorescent image shows nanoparticle distribution across both brain and spinal cord ROIs (FIGS. 6D, 6I). Nanoparticle delivery was achieved on the ventral surface of the brain in isotonic conditions (FIG. 6E). A whole-body fluorescent image shows nanoparticle distribution in the brain ROI and a lack of visually detectable nanoparticles in the spinal cord ROI (FIG. 6F). Co-infusion of hypertonic solution into the cisterna magna did not propel lipidated nanoparticles (red) from the head to the lower spinal cord ROIs (FIG. 6G).

### Example 6

### Distribution of Small Molecules and Nanoparticles in Hypertonic aCSF

### Materials and Methods

10 μL isotonic (1×) and hypertonic (2×) artificial CSF (aCSF) was co-infused with various substances into the cisterna magna of healthy C57 mice. Following substance administration, mice were immediately placed in a whole-body fluorescent imaging system and maintained under isoflurane anesthesia for approximately 2 hours. Mice were imaged in regular intervals, and regions of interest (ROIs; head, lower spinal cord) were overlayed on images to enable quantification of nanoparticle distribution to different tissues of the CNS. Fluorescence measurements are reported as arbitrary units (AU). At the termination of the experiment, brain and spinal cord were extracted from each mouse for imaging with a fluorescent stereoscope. In some instances, tissues were left intact. In other instances, tissues were sliced into thick (1-2 mm) coronal or sagittal sections.

The model small molecule rhodamine B was co-infused with either isotonic or hypertonic aCSF. The brains and spinal cords were extracted approximately 2 hours after substance administration and imaged with a fluorescent stereoscope.

The model small molecule hydrazide (ALEXA FLUOR® 488 Hydrazide fluorescent tracer; Invitrogen by Thermo Fisher Scientific, Catalog No. A10436) and model 20 nm, 40 nm, or 100 nm polystyrene nanoparticles were co-infused with either isotonic or hypertonic aCSF. The brains and spinal cords were extracted approximately 2 hours after substance administration and imaged with a fluorescent stereoscope.

GFP-expressing XX MDA-MB-231 cells were infused into the cisterna magna of NOD scid gamma (NSG) mice to induce leptomeningeal metastasis. Once tumors were at an advanced stage, model polyester nanoparticles (PLA-PEG, blue) were administered intrathecally via the cisterna magna, utilizing isotonic (1× aCSF) media, and tissue samples were collected 2 hours later. Whole neuroaxis (denoted “bone in”) as well as tissue extracted from the neuroaxis (denoted “bone out”) were imaged with a fluorescent stereoscope.

### Results

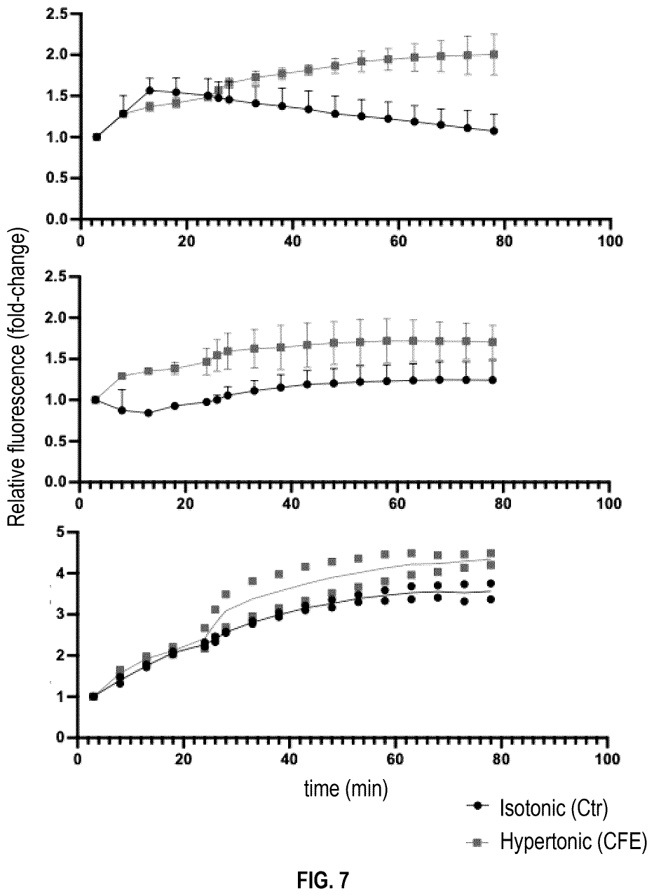

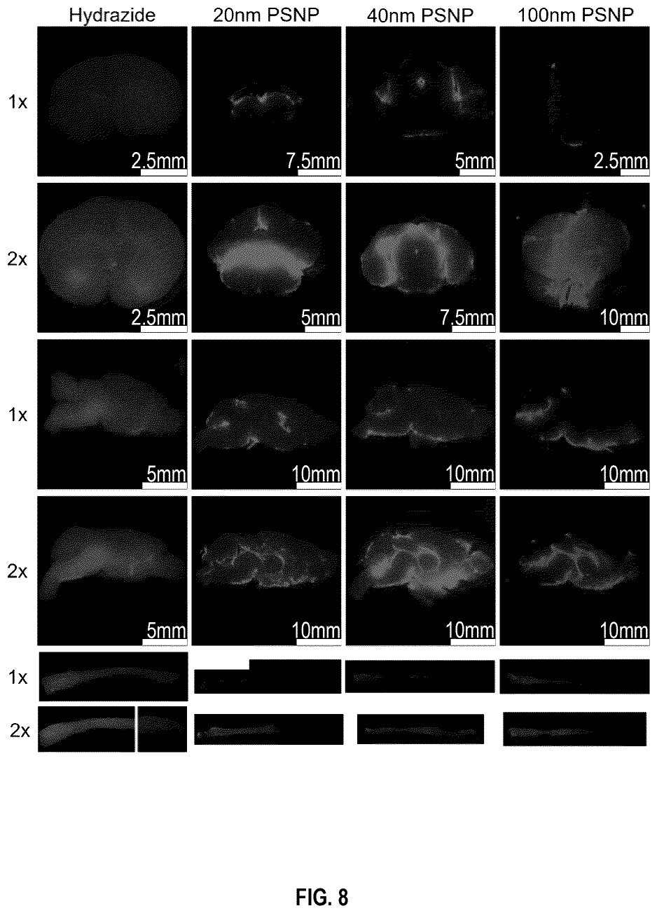

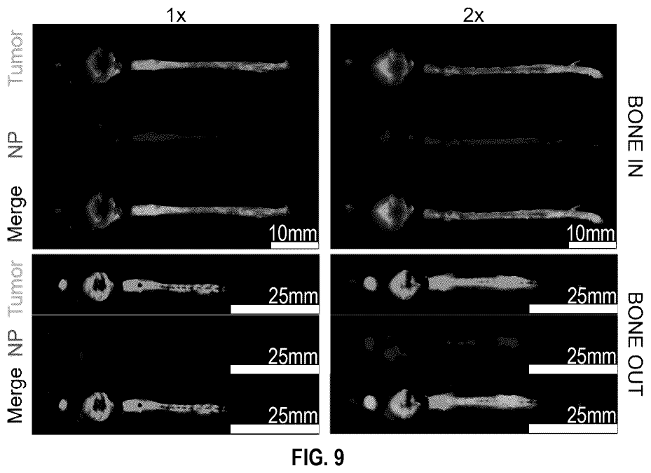

Co-infusion of hypertonic solution into the cisterna magna propelled Rhodamine B into brain and spinal cord ROIs (FIG. 7). Spatial distribution of various model compounds (hydrazide and 20 nm, 40 nm, and 100 nm polystyrene nanoparticles) in the brain and spinal cord is markedly enhanced by co-infusion with hypertonic aCSF compared to isotonic aCSF (FIG. 8). Intrathecally administered PLA-PEG nanoparticles (blue) distribute throughout the neuroaxis, localizing with leptomeningeal metastasis (green) in both “bone in” and “bone out” samples (FIG. 9).

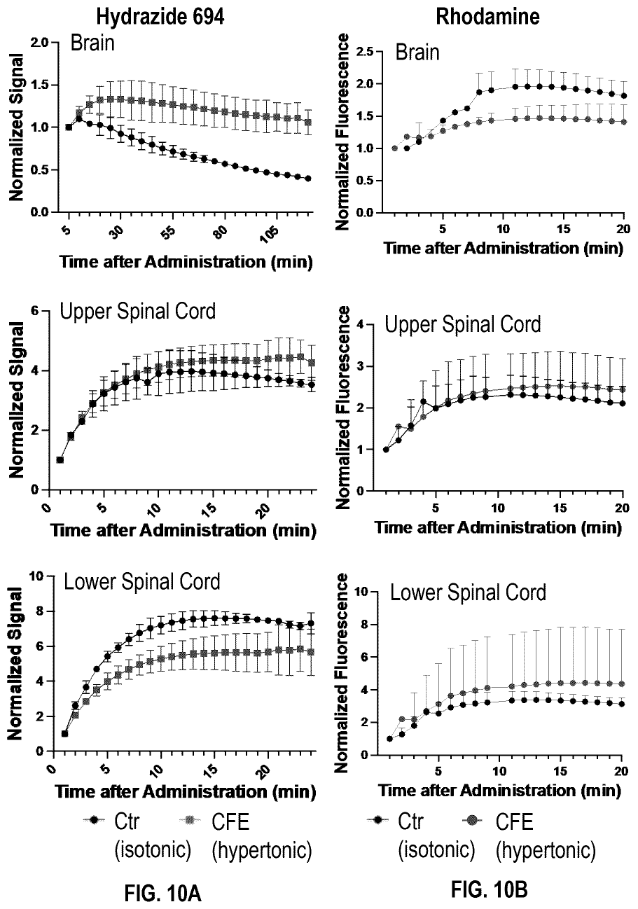

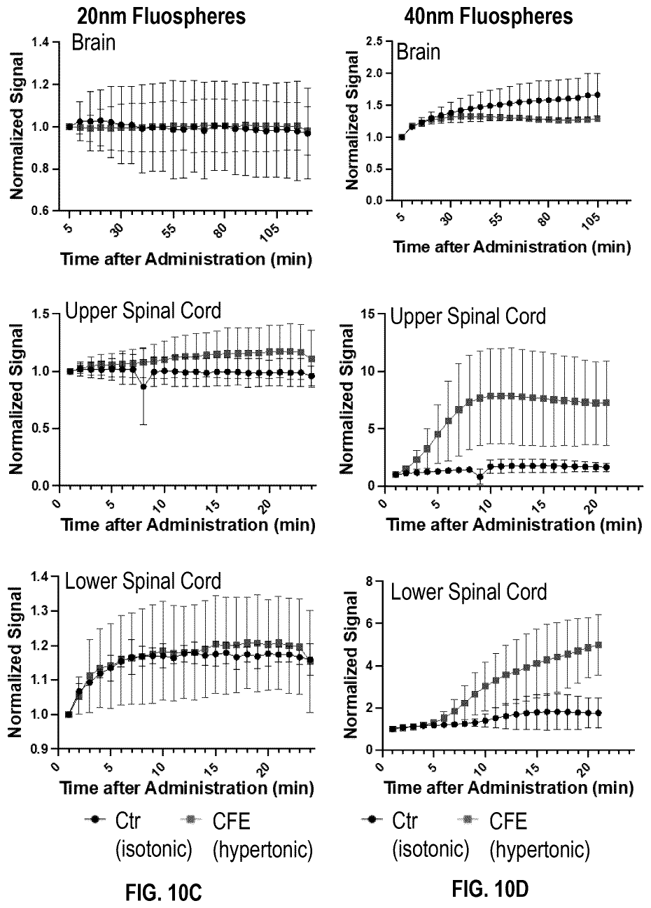

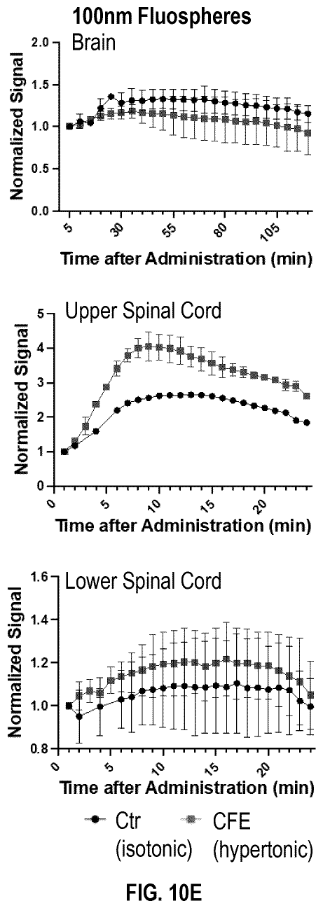

Co-infusion of hydrazide with hypertonic aCSF enhanced delivery to the brain ROI while modestly reducing delivery to the spinal cord ROI compared to isotonic control (FIG. 10A). Co-infusion of rhodamine with hypertonic aCSF enhanced delivery to the lower spinal cord ROI compared to isotonic control (FIG. 10B). Co-infusion of 20 nm polystyrene nanoparticles with hypertonic aCSF did not significantly impact signal in brain and spinal cord ROIs compared to isotonic control (FIG. 10C). Co-infusion of 40 nm polystyrene nanoparticles with hypertonic aCSF enhanced delivery to the upper and lower spinal cord ROIs compared to isotonic controls (FIG. 10D). Co-infusion of 100 nm polystyrene nanoparticles with hypertonic aCSF enhanced delivery to the upper and lower spinal cord ROIs compared to isotonic controls (FIG. 10E).

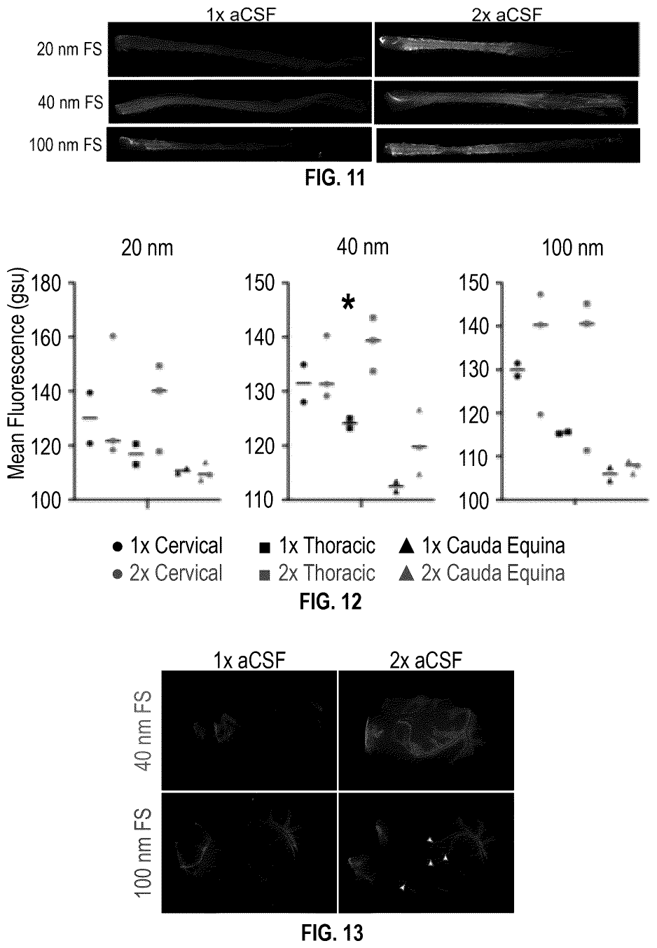

Co-infusion of 20, 40, and 100 nm polystyrene nanoparticles with hypertonic aCSF yielded markedly enhanced delivery of nanoparticles (FIG. 11). These data were quantified for dorsal vs ventral surfaces of the spinal cord in FIG. 12. Improvements in spinal cord delivery were observed with CFE for differently sized nanoparticles (FIGS. 11, 12).

Visualization of the lateral surface of the brain showed enhancement of nanoparticle localization with vasculature and perivascular tissue regions for hypertonic conditions compared to isotonic conditions (FIG. 13).

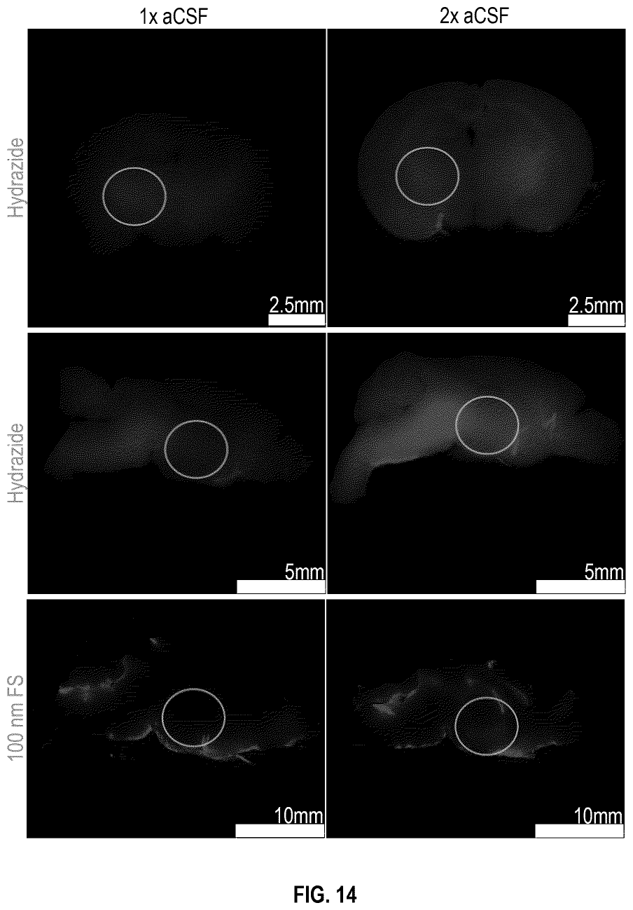

Visualization of coronal and sagittal brain slices showed enhancement of hydrazide and 100 nm polystyrene distribution in deep brain regions for hypertonic conditions compared to isotonic conditions. The ROI depicted here is the hypothalamus (FIG. 14).

### Example 7

### Role of pH in CFE

### Materials and Methods

10 μL isotonic aCSF with pH adjusted to acidic (pH 4.8) and physiologic (pH 7.0) conditions was co-infused with 100 nm polystyrene nanoparticles into the cisterna magna of healthy C57 mice. Following substance administration, mice were immediately placed in a whole-body fluorescent imaging system and maintained under isoflurane anesthesia for approximately 2 hours. Mice were imaged in regular intervals, and regions of interest (ROIs; head, lower spinal cord) were overlayed on images to enable quantification of nanoparticle distribution to different tissues of the CNS. Fluorescence measurements are reported as arbitrary units (AU). At the termination of the experiment, brain and spinal cord were extracted from each mouse for imaging with a fluorescent stereoscope, slicing tissue into thick sections (1 mm) sections.

ALEXA FLUOR® hydrazide was administered to mice in a standard infusion (1× aCSF) or under CFE conditions (2× aCSF) with variable pH (acidic pH 4.8 or physiologic pH 7.0). n=3 mice for each group. Spatial distribution of water-soluble tracer (ALEXA FLUOR® hydrazide) across the brain (left to right on coronal sections) was assessed.

### Results

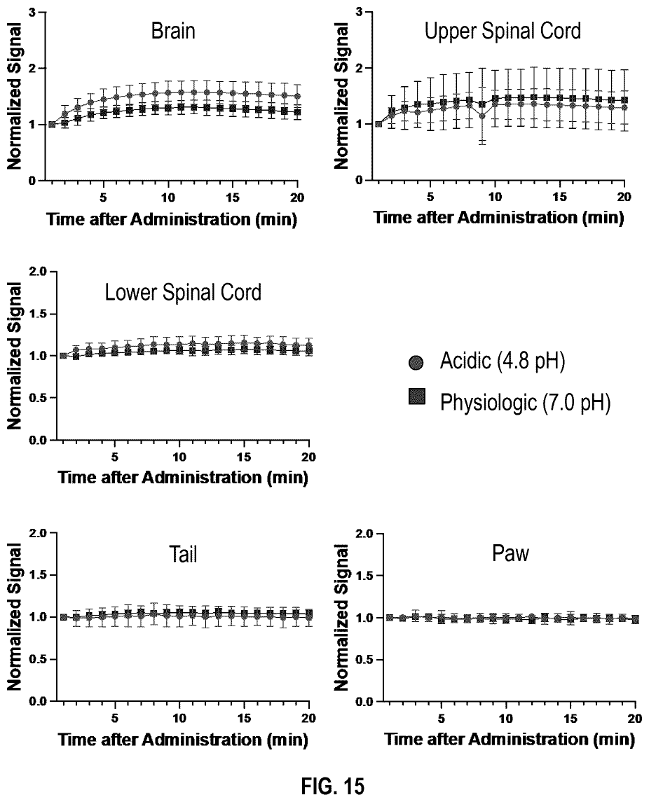

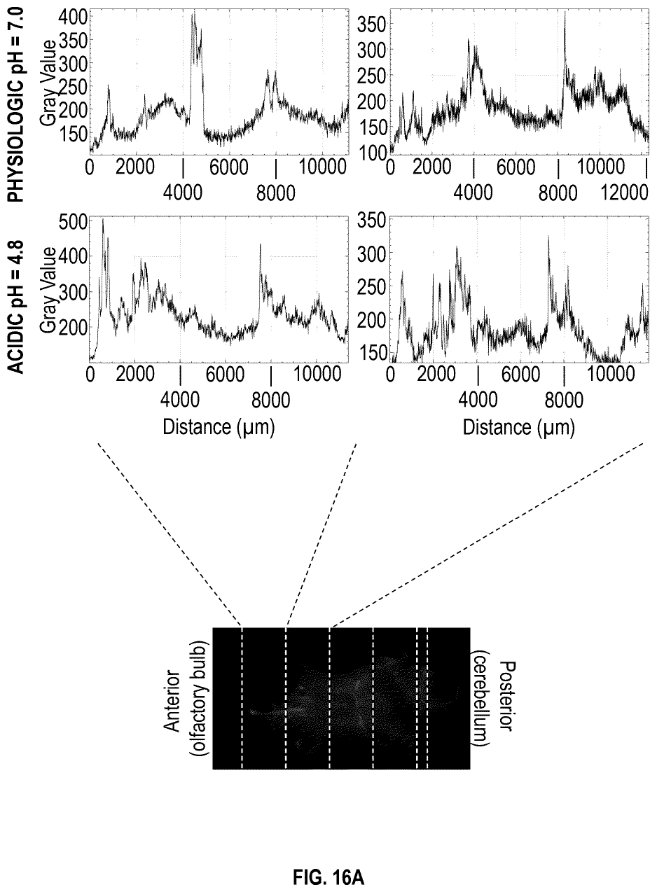

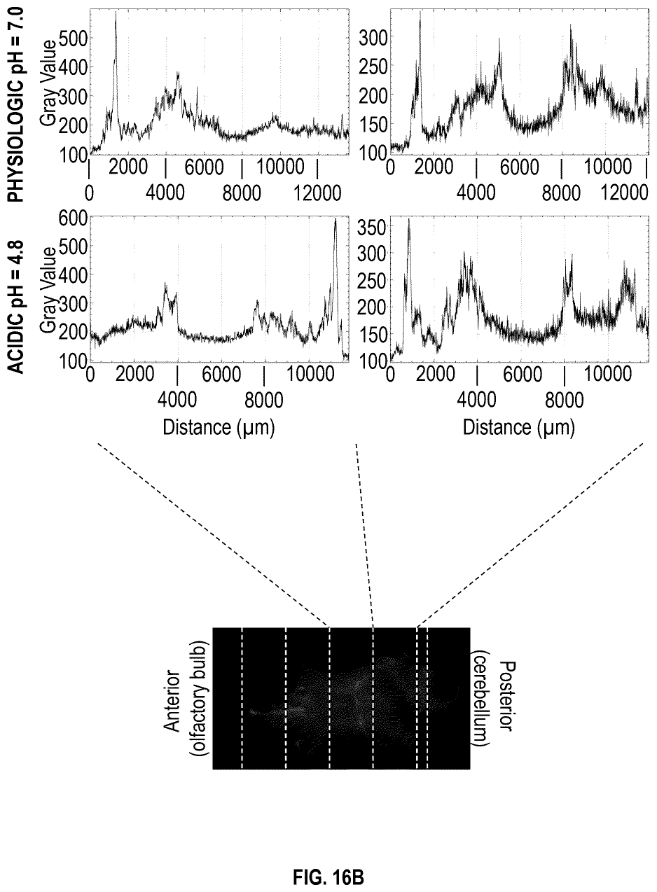

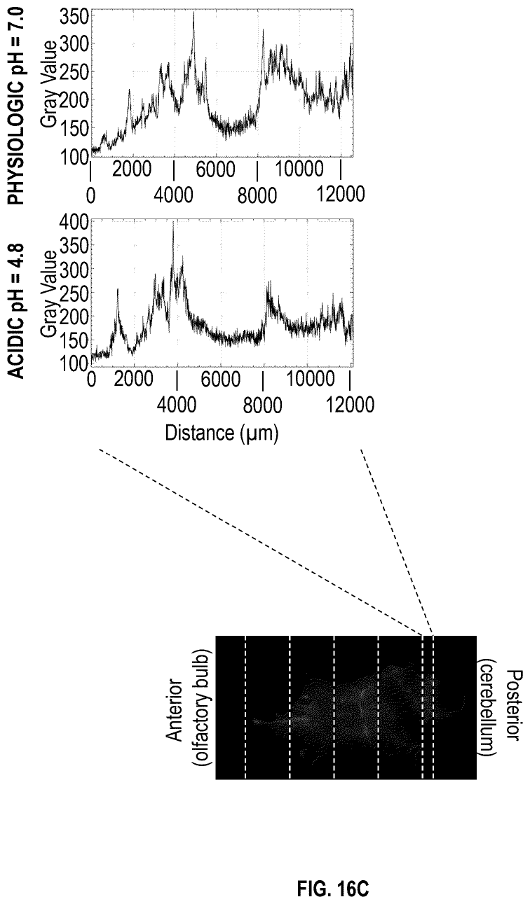

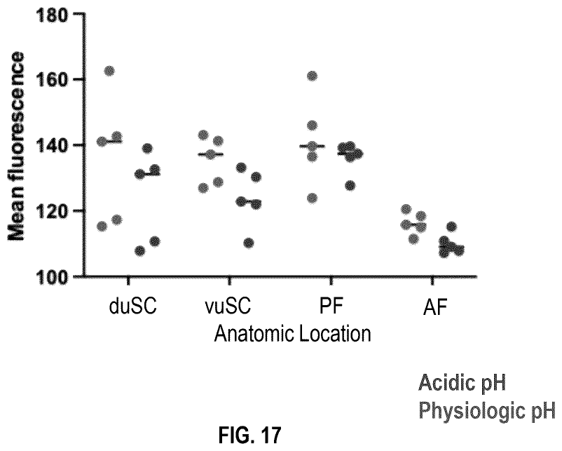

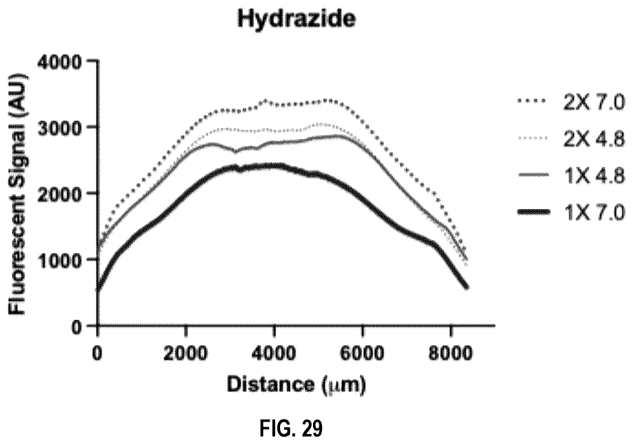

Nanoparticle signal in the lower spinal cord tended to be higher for acidic conditions compared to physiologic controls (FIG. 15). Line plot profiles were generated from sagittal tissue sections to quantify nanoparticle distribution along the anterior-posterior axis of the brain (FIGS. 16A-16C). Any differences between acidic and physiologic conditions were minor or inconsistent between subjects. There was a minor tendency for acidic conditions to produce an increase in delivery to the dorsal upper spinal cord (duSC), ventral upper spinal cord (vuSC), posterior fossa (PF), and anterior fossa (AF) compared to physiologic controls (FIG. 17). These differences were not statistically significant. The difference in nanoparticle distribution between acidic and physiologic pH conditions was modest for a 1× isotonic aCSF infusion. pH had a minor effect on substance administration by this route. Subsequent experiments showed that CFE conditions (2× aCSF), acidic pH (pH=4.8), and CFE and acidic pH together provide various levels of improvement in the distribution of water-soluble tracer across the brain, demonstrating the direct effect of pH manipulation and tonicity manipulation on distribution (FIG. 29).

### Example 8

### CSF Enhancement as a Therapeutic Strategy in TBI

The mechanisms by which traumatic brain injury (TBI) contributes to later in life neurodegeneration are incompletely understood but involve aberrant molecular and cellular processes leading to neuronal, axonal and/or synaptic degradation, vascular injury, mitochondrial dysfunction, neuroinflammation, as well as accumulation of metabolic waste products, deleterious signaling molecules and toxic proteins (21, 22, 109, 110).

TBI is one of the most common types of injuries sustained by military personnel (8,9). The risk of developing neurodegenerative disease including dementia due to TBI is twice that of the general population (40). TBI is a major health burden (8,9) and an important risk factor for several progressive neurodegenerative disorders, including Parkinson's disease (PD), amyotrophic lateral sclerosis (ALS), Alzheimer's disease (AD), and Alzheimer's disease-related dementias (ADRDs) such as frontotemporal dementia (FTD) (10-20).

Preclinical studies are required to define the pathogenic mechanisms linking TBI to acute and chronic neurodegenerative disease development to devise novel treatment strategies to promote recovery and prevent age-related neurodegenerative disease in veterans. CFE is a novel approach to safely manipulate physiology of CSF in a highly controlled fashion, allowing restoration of CNS fluid homeostasis after injury. Impact from the work described herein will include: (a) development of fundamentally new research tools to more precisely control the microenvironment and physiology of the CNS and facilitation of CNS drug delivery; (b) novel insights into mechanisms driving poor outcomes from acute and chronic TBI; and (c) therapeutic approaches that will benefit those affected by impact-associated neurological diseases.

Accumulation of metabolic waste products or deleterious signaling molecules within fluids of the central nervous system (CNS) is proposed to be a driving factor of traumatic brain injury (TBI) contribution to later in life neurodegeneration. This “toxic CSF” theory is supported by observations that enhancing cerebrospinal fluid (CSF) clearance is neuroprotective in preclinical models of CNS injury (23,24), inflammation (25), and neurodegeneration (26,27). Additional evidence that toxic CSF components can promote neurodegeneration is that CSF from amyotrophic lateral sclerosis (ALS) patients injected into the CSF of mice induces motor deficits, which can be prevented by filtering the CSF prior to transplant (26).

Under normal physiological conditions, CSF secreted by the choroid plexus flows from the cerebral ventricles into the subarachnoid space, from where it enters the parenchyma via artery- and arteriole-adjacent perivascular spaces (PVS) (see graphical abstract of reference (103)). PVS facilitate CSF exchange with interstitial fluid and eventual clearance into the periphery via cervical lymphatics (28). This network of fluid clearance pathways, recently termed the “glymphatic” system (see FIG. 1 of reference (104)), facilitates CSF turnover and maintenance of brain homeostasis (29). Importantly, the glymphatic system is disrupted following TBI (30). Enhancement of glymphatic function is neuroprotective in a murine model of TBI (23) and has also been shown to be therapeutically effective in other models of neuroinflammation and/or neurodegeneration (24-27).

CSF flow enhancement (CFE) refers to manipulations that augment the production, distribution, clearance, or composition of CSF. Two major gaps prevent broad application of this field as a method to treat or prevent neurodegeneration: first, how is CFE best implemented in a manner that is both safe and effective?Second, what are the mechanisms by which CFE is neuroprotective in different injury contexts, for example, in acute vs. chronic injury?

Current approaches for CFE have been studied in individual reports only and include systemic manipulations that are physiological (e.g., sleep, exercise, and mechanical), pharmacological (small molecule adrenergic agonist), or biophysical (systemic administration of hypertonic saline or mannitol). Disclosed here in is an exciting new approach for achieving local CFE by which intrathecal infusion of hypertonic solutions generates rapid production of CSF by the choroid plexus to effectively “wash” freshly secreted CSF through the brain parenchyma via glymphatic and other clearance routes (FIGS. 1C, 1D). The use of CFE following TBI represents a new field of study and a major conceptual innovation in how to treat or prevent injury-associated neurodegeneration by which the underlying physiological processes that are damaged by TBI will be augmented.

The present disclosure contributes to the development of translatable therapies for TBI. Further, the present disclosure indicates that CFE ameliorates injury-induced neurodegeneration due to enhanced clearance of toxic metabolic byproducts via the glymphatic system.

### Identify Mechanisms of Waste Clearance Facilitated by CFE in the Healthy Brain

Local CFE will enhance CSF egress from the CNS while maintaining integrity of the BBB compared to systemic CFE. Clinically, intravenous hypertonic saline is used to reduce intracranial pressure, in part by opening the blood brain barrier (BBB) to permit egress of fluids of the CNS to the periphery, and it has also been claimed that this reduces neuroinflammation (31), but there is a paucity of studies directly evaluating these claims. Preclinically, it has been shown that systemic administration of a pan-adrenergic agonist cocktail partially restores glymphatic function and is neuroprotective in murine TBI (23). Studies disclosed herein demonstrate that intrathecal infusion of hypertonic solutions is an alternative, safe, and tunable method for CFE that circumvents systemic permeabilization of the BBB and also provides an opportunity for drug delivery (FIGS. 1C, 1D). These three methods therefore provide complementary approaches for achieving CFE and will be compared head-to-head using intravital microscopy and other dynamic imaging tools to comprehensively assess how different methods for CFE alter fluid egress pathways, CSF composition, and vascular permeability.

### Optimize Time-Course of CFE to Prevent Acute and Chronic Neurodegeneration in Murine TBI

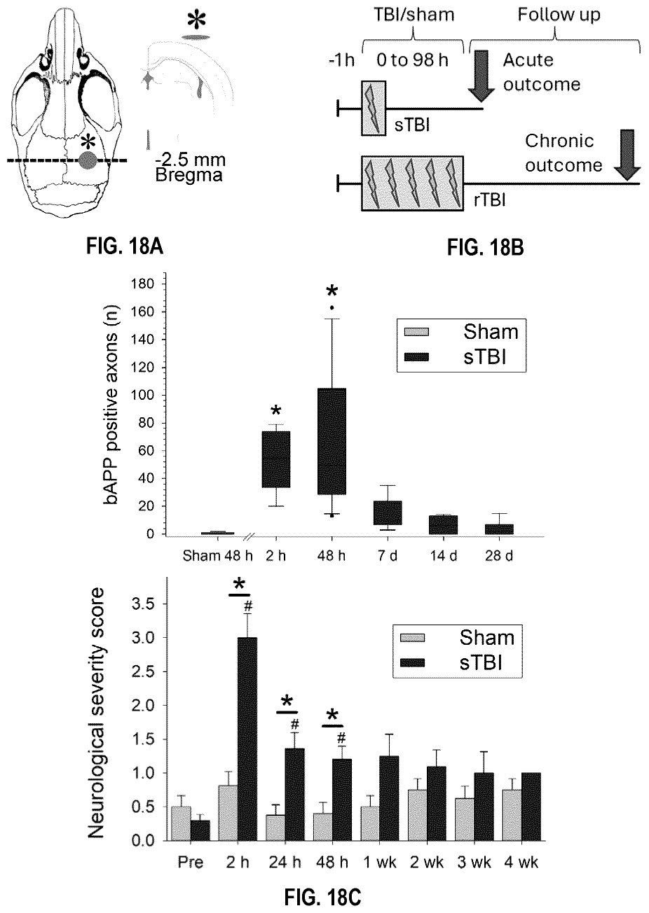

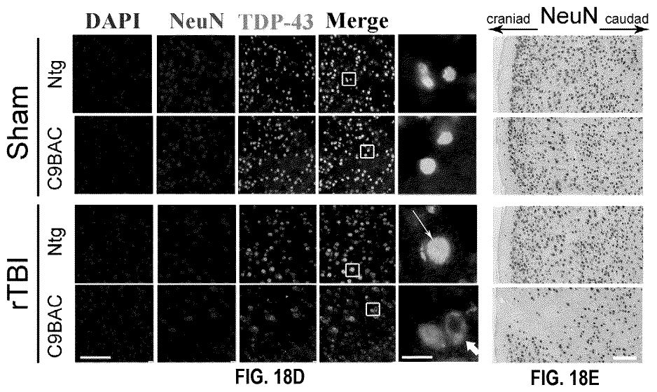

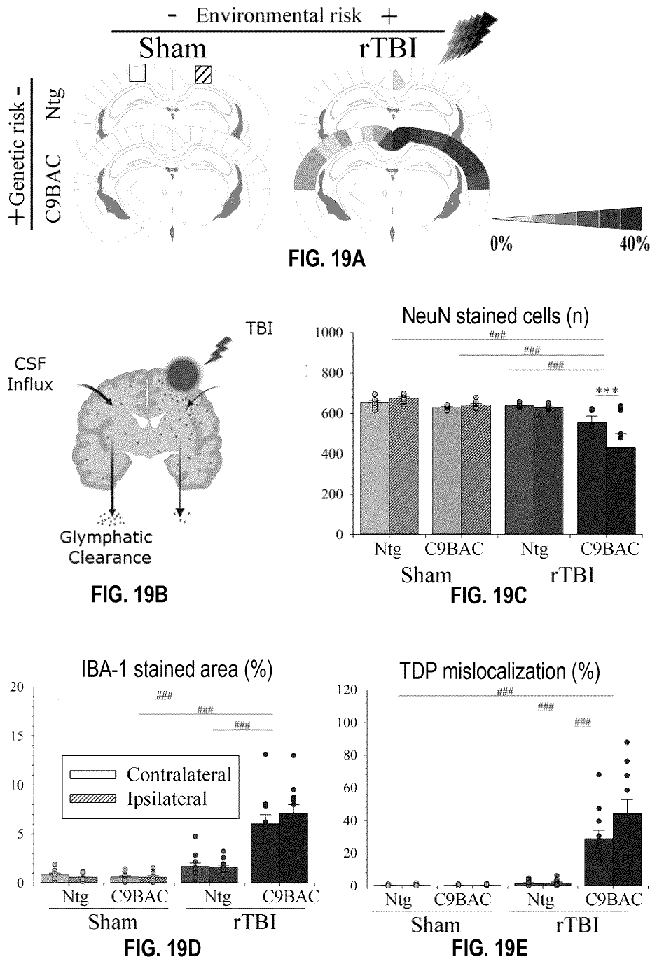

CFE will alleviate neuronal loss and behavioral deficits acutely following acute TBI (32,33) as well as prevent chronic neurodegeneration in an innovative model of TBI-induced frontotemporal dementia (FTD). In addition to traditional severe TBI (sTBI) models, a uniquely useful model of brain injury-induced FTD has been developed (FIGS. 18A-18C, 19C, 19D, 19E), whereby mild recurrent TBI (rTBI) yields extensive, delayed neuronal and axonal loss, persistent neuroinflammation, and TDP-43 mis-localization in transgenic C9orf72 mice; this neurodegeneration is not seen in non-transgenic or non-injured controls (33). The ability of CFE to ameliorate acute and chronic injury processes will be studied in established models of sTBI and rTBI, including detailed behavioral, cellular, and pathological analyses to comprehensively evaluate the potential of CFE to ameliorate these injury processes.

### Example 9

### CSF Flow Augmentation to Mitigate TDP-43 Pathology and TBI-associated Neurodegeneration

Traumatic brain injury (TBI) represents a major public health problem affecting more than 10 million people worldwide each year (108). TBI is a leading cause of adult death and disability worldwide. It has been estimated that annually 150 to 200/1,000,000 people become disabled as a result of brain trauma, and TBI was declared a major public health problem by the National Institutes of Health in 1999 (105, 106, 107).

Amyotrophic lateral sclerosis (ALS) and frontotemporal dementia (FTD) are uniformly lethal neurodegenerative diseases that share clinical and pathological findings. Epidemiological evidence suggests that traumatic brain injury (TBI) is an important risk factor for progressive neurodegenerative disorders, including FTD and ALS. Recurrent TBI (rTBI) is sufficient to induce chronic neurodegeneration in absence of acute tissue pathology in a mouse ALS/FTD model, thus mimicking the human circumstance and providing a unique model in which therapeutic interventions prior to onset of axonal loss and neuroinflammation can be assessed.

Impaired cerebrospinal fluid (CSF) flow is both a feature of and a contributor to brain injury, and manipulation of CSF flow or exchange may provide an important therapeutic opportunity for intervention. The overarching hypothesis of this proposal is that potentiating CSF flow augments clearance of neurotoxic protein deposits and inflammatory cytokines from the brain parenchyma after injury, which, according to the present disclosure, is predicted to prevent neurodegenerative disease onset after repetitive traumatic brain injury.

The contemplated high translational value studies will dissect the contribution of compromised CSF flow to neurodegeneration and behavioral dysfunction after TBI and in the context of C9orf72 ALS/FTD, and therefore will have the potential to transform the field understanding impact related and neurodegenerative disease pathology.

The most common cause of ALS and FTD is a hexanucleotide repeat expansion (HRE) in the C9orf72 gene. People carrying the mutation can have up to thousands of repeats, while healthy individuals have fewer than 30. The mechanisms whereby these expansions cause neurodegeneration are not well delineated but include C9orf72 haploinsufficiency, as well as accumulation of neurotoxic hexanucleotide transcripts and poly-dipeptides translated from the HRE.

Strong epidemiological data suggests that TBI is a risk factor for ALS and FTD, but the mechanisms by which TBI causes ALS/FTD are not well delineated. Persistent or irreversible cytoplasmic accumulation of TAR DNA-binding protein 43 kDa (TDP-43) is a major pathological event in TBI and associated neurodegenerative diseases; it is present in >95% of ALS cases and in all cases of C9orf72 FTD. While TDP-43 is an essential nuclear protein regulating RNA metabolism, in pathological conditions, cytoplasmatic mislocalization of TDP-43 in neurons and glial cells leads to loss of TDP-43 function resulting in abnormal processing of neuronal RNA targets but also dysfunctions in mitochondria and nucleo-cytoplasmic transport due to toxic gain of function of TDP-43 aggregates.

Established mouse models of rTBI across a range of injury severities mimic diverse aspects of human TBI-associated pathology (see, for example, (32, 38, 85)). rTBI causes several pathological findings shared with ALS/FTD, including widespread neuronal and axonal degeneration, neuroinflammation, and mislocalization and deposition TDP-43 (85). More recently, rTBI was established as sufficient to trigger ALS/FTD-like neuropathology including TDP-43 mislocalization and behavioral deficits in mice carrying a C9orf72 HRE (33).

TDP-43 is detected in the perivascular space as well as in the CSF of ALS patients, and emerging data suggests that the release of TDP-43 into the CSF is an important pathway by which pathological TDP-43 spreads and accelerates disease in affected individuals. Moreover, transgenic mice expressing human mutant TDP-43 with pathological accumulation of TDP-43 develop an ALS-like phenotype and have significantly disrupted glymphatic function. TBI has been shown to result in long-lasting reduction in the glymphatic flow leading to impaired clearance of abnormal protein deposits in the parenchyma; normalizing glymphatic drainage after TBI improves functional recovery and attenuates the accumulation of pathological proteins such as hyperphosphorylated Tau; however, whether augmenting CSF flow yields clearance of TDP-43—and whether this mitigates neurodegeneration in susceptible individuals—remains unknown.

ALS and FTD are uniformly lethal neurodegenerative diseases. TBI accelerates disease penetration, onset, and course in carriers with an ALS/FTD gene defect. According to the present disclosure, CSF flow augmentation (e.g., cerebrospinal fluid flow enhancement (CFE)) may be a novel approach to prevent pathological TDP-43 accumulation and neurodegeneration following rTBI in a murine model of ALS/FTD.

Frontotemporal dementia (FTD) and amyotrophic lateral sclerosis (ALS) are two devastating, uniformly lethal neurodegenerative diseases that share clinical, genetic, and pathological findings. A GGGGCC (G4C2) hexanucleotide repeat expansion (HRE) in the first intron of the chromosome 9 open reading frame 72 (C9orf72) gene is the most common cause of both ALS and FTD (c9ALS/FTD) (54-56). This mutation can have up to thousands of repeats (57). How these expansions cause neurodegeneration is not well understood; possible mechanisms include C9orf72 haploinsufficiency and neurotoxicity from the hexanucleotide transcripts and poly-dipeptides (DPR) translated from the HRE (58). Strikingly, typical pathology may predate disease onset by years; disease onset and duration are variable (59-61). These observations indicate that environmental stimuli modulate disease pathogenesis and open the possibility of therapeutic intervention prior to phenotypic onset.

Traumatic brain injury (TBI) has been linked to an increased risk for ALS/FTD. Epidemiological evidence suggests that TBI is an important risk factor for progressive neurodegenerative disorders (62), including FTD and ALS (62-68). At the cellular level, TBI and ALS/FTD are both characterized by widespread axonal degeneration as well as mislocalization and deposition of TDP-43, suggesting a mechanistic link (69-71). It has been demonstrated that mice carrying a C9orf72 HRE develop neurodegenerative pathology following mild, rTBI. Importantly, this model of rTBI-induced ALS/FTD produces chronic neurodegeneration in absence of acute tissue pathology, thus mimicking the human circumstance and providing a unique model in which therapeutic interventions prior to onset of axonal loss and neuroinflammation can be assessed. Waste clearance pathways in the central nervous system (CNS) may be an important therapeutic target in ALS/FTD.

Impaired cerebrospinal fluid (CSF) flow is both a feature of and a contributor to brain injury (23, 72, 73), and growing evidence shows that manipulation of CSF flow or exchange may provide an important therapeutic opportunity for intervention. Under physiological conditions, CSF is partially or fully drained by outflow pathways that include the meningeal and cervical lymphatic vessels (74), which return fluid through the thoracic duct to the venous circulation (75). Blockade of meningeal or cervical lymphatic vessels accelerates the deposition of pathological proteins such as amyloid-β, Tau protein, and α-synuclein in rodent disease models (76-78). Transfer of CSF obtained from ALS patients into the cisterna magna of healthy mice yielded motor deficits (26); preclinically, CSF exchange has been shown to be neuroprotective in murine models of multiple sclerosis (25), Alzheimer's disease (27), and TBI (23); clinically, CSF exchange has been shown to ameliorate symptoms of Guillain-Barre syndrome (79).

Although CSF augmentation has been shown to attenuate neuroinflammation and reduce concentrations of Tau protein after TBI, it is unknown whether this approach can prevent the accumulation of neurotoxic TDP-43 and how this relates to neurodegeneration in general and in ALS/FTD in particular.

Additionally, the methods currently used for enhancing exchange of CSF or augmenting CSF flow require lumbar or ventricular catheterization, which is highly invasive and may not be translatable to pre-symptomatic (but genetically susceptible) individuals. Preclinical and clinical observations described above provide a strong rationale for the belief that that potentiating CSF flow augments clearance of neurotoxic protein deposits and inflammatory cytokines from the brain parenchyma after injury, which is believed, according to the present disclosure, to prevent neurodegenerative disease onset after rTBI.

Neurodegeneration, neuroinflammation, and pathological TDP-43 accumulation and mislocalization are hallmarks of TBI and most cases of ALS and Tau-negative cases of FTD (80-84). Recent work showed for the first time that TBI can trigger these pathologies in a model of c9ALS/FTD (33). In this model, rTBI causes widespread microglial activation and reduced neuronal density that is associated with loss of histological markers of axonal and synaptic integrity as well as profound neuronal TDP-43 mislocalization in the cerebral cortex of transgenic C9orf72 mice (C9BAC), that is not observed in non-transgenic rTBI, and sham mice (FIGS. 18A-19E) (33). The approach for CSF flow augmentation relies on homeostatic capacity of the choroid plexus, which responds dynamically to even modest perturbation of pH and solute concentration. Introduction of salt into the CSF yields a dramatic generation of fluid that subsequently flushes through the perivascular space, which has been shown to propel substances throughout the brain and into CSF- and glymphatic clearance pathways (FIGS. 1C, 18A-18E, 19A-19E). The approach has been extensively optimized in mice, evaluating a range of infusion volumes (2-20 L), tonicity (1×-8× concentrated artificial CSF, 1×-5× concentrated phosphate buffered saline (PBS)), and speed of infusion (slow versus fast bolus), selecting a 10 μL volume with 2× aCSF delivered via slow bolus. For the studies contemplated herein, the established innovative mouse models of TBI-induced ALS/FTD will be combined with this powerful method for CSF augmentation (FIGS. 18A-19E) to: (a) probe histological hallmarks of rTBI-induced brain neurodegeneration, (b) assess motor strength and coordination in an interventional context, and (c) perform unbiased quantitative proteomics that will begin to untangle the mechanisms by which CSF flushing is neuroprotective. Experiments will be conducted in male and female mice carrying a truncated (exons 1-6) human mutant C90RF72 gene with approximately 500 repeats of the G4C2 motif (C9BAC) (33). Augmentation of CSF flow is produced by percutaneous infusion of 10 μL of hypertonic artificial CSF (aCSF) via the cisterna magna (4). Mice will receive either aCSF with standard composition (control) or with the concentration of solutes increased proportionally achieve 2× total tonicity, yielding an approximately 20% increase in CSF tonicity if well-mixed. Ten mice per experimental group and time point will be used. All animals will be socially housed with same-sex mice (n=4 per cage) on 12-h light/dark cycle with food and water ad libitum. Animals will be followed up for 3 months after TBI at which point brains will be removed for analyses. Data will be analyzed using two-way analysis of variance (ANOVA) or generalized linear models that account for treatment and sex, as appropriate. A two-sided p<0.05 adjusted for multiple comparisons will be considered significant. Collectively, these studies will advance understanding of the mechanisms and potential clinical impact of CSF augmentation in brain injury research (33,38, 85-87).

ALS and FTD are uniformly lethal neurodegenerative diseases. Epidemiological evidence indicates that TBI accelerates disease onset and course in carriers with an ALS/FTD gene defect. According to the present disclosure, CSF-flow augmentation is a novel approach to mitigate pathology. The contemplated experiments will identify cellular and molecular response pathways to glymphatic compromise. Importantly, the contemplated studies investigate CSF-flow augmentation as a novel approach to mitigate the devasting consequences of this and other TBI-associated neurodegenerative diseases including Alzheimer's disease (AD) and related dementias (ADRD). The investigations described below are contemplated to determine the physiological links between CSF flow, TDP-43 clearance, and TBI-associated neurodegeneration using our established mouse disease models and an innovative new method for enhancing CSF flow through perivascular spaces. According to the present disclosure, it is hypothesized that potentiating CSF flow augments clearance of neurotoxic protein deposits and inflammatory cytokines from the brain after injury, which is predicted to prevent neurodegenerative disease onset after rTBI. This study will provide novel insight into the links between CSF flow dynamics, clearance of abnormal protein deposits, and if CSF flow augmentation may present a novel approach to prevent chronic neurodegeneration in disease.

Test Whether CSF Augmentation Decreases Susceptibility to ALSIFTD Following rTBI

CSF flow augmentation is hypothesized herein to prevent TBI-associated ALS/FTD pathology. Injuries in an established murine model of rTBI-induced ALS/FTD will be generated. Following injury, mice will receive infusions of isotonic artificial CSF (control) or hypertonic artificial CSF (treatment) into the cisterna magna, which existing data demonstrate is an effective method for enhancing CSF distribution within and clearance from the brain. Neuronal and axonal integrity, TDP-43 mislocalization, and neuroinflammation longitudinally will be quantified for up to 3 months after rTBI. Western immunoblotting (WB) and quantitative polymerase chain reaction (qPCR) will characterize TDP-43 expression longitudinally. Behavioral function will be serially assessed using a battery of motor tests up to 3 months after rTBI.

At 1 and 3 months after rTBI, histological markers in a murine model of rTBI-induced ALS/FTD will be assessed. Two groups will be studied: control (intracisternal infusions of isotonic aCSF) and treatment (intracisternal infusion of hypertonic aCSF). Neuronal (e.g., NeuN), axonal (e.g., SMI-312), microglial (e.g., Ibal), and astroglial (e.g., GFAP) markers will be included using established protocols. To compare the extent of neuronal and axonal loss and neuroinflammation on immunostaining, threshold-based quantification of the area stained with axon, neuron, and glial markers will be used as described (38). The number neurons showing nuclear loss, cytoplasmatic mislocalization, and cytoplasmatic aggregation of TDP-43 will be quantified using immunostaining for TDP-43 pathology with validated anti-TDP-43 (including phosphospecific) antibodies as described (38). Analyses will be conducted at Bregma −2.5 mm (impact center, FIG. 18A). WB and qPCR will be used to characterize TDP-43 expression at time points corresponding to the histological analyses in the ipsilateral and contralateral cortex. Neurological evaluations of mice will be serially assessed (1 week, 1, 2, and 3 months after rTBI) by the grip strength test and accelerating (4-40 rpm) Rotarod test for motor function (33).

In murine TBI, long-lasting reduction in the glymphatic flow has been observed; normalizing glymphatic drainage after TBI improves functional recovery (23). Infusion of hypertonic aCSF into the cisterna magna of healthy rodents significantly improved trans-parenchymal flow of CSF as well as distribution and clearance of infused substances (FIGS. 19B, 1E, 5A), demonstrating feasibility of CSF flow augmentation. According to the present disclosure, CSF-flow augmentation is expected to attenuate neuronal loss, neuroinflammation, and nuclear loss and mislocalization of TDP-43 as well as improve motor deficits compared to controls. CSF-augmentation (e.g., cerebrospinal fluid flow enhancement (CFE)) may attenuate neuronal loss and neurological deficits without altering the extent of TDP-43 mislocalization, indicating that clearance of solutes other than TDP-43 improves neurodegeneration in the model. This would be an important observation for the scientific community as perivascular TDP-43 accumulation has been proposed as a driver of impaired glymphatics and pathology in ALS (88). Profile the temporal dynamics of protein and chemokine clearance following CSF augmentation

Following CSF augmentation (e.g., cerebrospinal fluid flow enhancement (CFE)), it is hypothesized herein that a reduction of abnormal protein deposits, including TDP-43 as well as inflammatory chemokines/cytokines, will be detected in the brain parenchyma after rTBI. Using unbiased quantitative proteomics and cytometric bead array assays, the composition and level of parenchymal proteins including TDP-43 as well as cytokines after CSF-augmentation will be detected in an established rTBI-induced ALS/FTD paradigm.

As described above, two groups of mice will be treated with either isotonic aCSF or hypertonic aCSF. For unbiased assessment of parenchymal protein changes after CSF-flow augmentation, quantitative proteomics will be conducted as guided by and through the University of Massachusetts Chan Medical School Mass Spectrometry Facility. Samples will be collected from the ipsilateral injured and contralateral uninjured cortex at 24 hours, 1 month, and 3 months after rTBI. To probe changes in levels of IL-1β in the brain as mediators of the neuroinflammatory response, cytometric bead assays will be used according to established methodology (85) in samples corresponding to the proteomic analyses (87).

As described above, rTBI causes widespread microglial activation in C9BAC mice. According to the present disclosure, it is hypothesized that CSF-flow augmentation attenuates the accumulation of abnormal proteins including TDP-43. TDP-43 inclusions (aggregates) activate the NLRP3 (NOD, LRR, and pyrin domain-containing protein 3) inflammasome in primary microglial cultures, resulting in increased production of interleukin-1l (IL-1) (89). IL-13 is a pleiotropic cytokine that can activate microglia and promote pathology in several neurodegenerative diseases and TBI (90,91). According to the present disclosure, it is hypothesized that in parallel with microglial activation, IL-1β (and other cytokines) will increase after rTBI (91,92); and that this response is suppressed by CSF-flow augmentation (23). The results will help to interpret the results from immunostaining experiments discussed above with respect to potential changes in the neuroinflammatory response after CSF-flow augmentation.

### Example 10

### Treatment of Leptomeningeal Metastasis (LM)

Medulloblastoma (MB) is the most common malignant childhood brain tumor and disproportionately impacts patients and families due to a lack of treatment options and a high rate of recurrence (1,2). MB exhibiting leptomeningeal metastasis (LM) poses a particular challenge. Because LM cannot be surgically resected and is often unresponsive to systemic chemotherapy, patients are generally treated with high dose chemoradiotherapy (CRT), which yields severe treatment-associated sequelae and very poor long-term outcomes (2). Recurrent LM is untreatable (3) and drug delivery remains a primary obstacle to the treatment of LM.

Nanomedicine offers new opportunities for drug delivery the central nervous system (CNS), for example, to metastatic cancer. Leptomeningeal metastasis (LM) resides within the sympathoadrenal system (SAS), a CNS region that is poorly vascularized and difficult to access by systemic routes. Biodegradable nanoparticle (NP) systems (about 100-400 nm in diameter) for intrathecal (IT) administration to slowly release small molecule drugs (4,5).

Recurrent LM is considered in the art to be untreatable; the method and compositions disclosed herein advance new therapeutics to prolong survival and minimize relapse. This disclosure represents a major conceptual shift to drug delivery in medulloblastoma (IB), advancing new approaches with strong potential for clinical translation.

Test Whether Higher Aspect Ratios Will Enhance NP Localization with LM

In accordance with the data disclosed herein, higher aspect ratios are expected to enhance NP localization with LM. NP geometry, including both size and aspect ratio, is known to be one of the most important factors influencing biodistribution and cellular-level deposition for the IV route (6), including in cancer (7), but has never been studied for the IT route. The data disclosed herein support the expectation that narrow (torpedo-like) shapes are better able to enter the brain parenchyma. The impact of NP size (10-100 nm) and aspect ratio (1:1-1:50 short:long axis) on the ability of NPs to penetrate metastatic lesions will be systematically studied.

Test Whether Enhancing Movement of CSF Through the SAS Will Enhance NP Localization with LM

In accordance with the present disclosure, enhancing movement of CSF through the SAS is hypothesized to enhance NP localization with LM. As disclosed herein, a powerful new approach for enhancing drug delivery to the CNS after IT administration comprises a mild increase in infusion solution tonicity that drives CSF—as well as NPs—into the parenchyma.

### Evaluate the Ability of Shape- and Delivery-Optimized NPs to Improve Treatment of LM Relative to Baseline Controls and Freely Administered Drug

In accordance with the present disclosure, it is believed that shape- and delivery-optimized NPs will improve treatment of LM relative to baseline controls and freely administered drug. The ability of shape- and delivery-optimized NPs to improve treatment of LM relative to baseline controls and freely administered drug will be evaluated. Drug distribution and LM-specific efficacy in mice bearing orthotopic MB exhibiting LM will be examined, focusing on 2-4 approaches identified above.

### Example 11

### CFE can Reduce Neuroinflammation in Injured Mouse Brain.

### Methods

Male C57BL/6 J mice (The Jackson Laboratory) were socially housed with same-sex mice on a 12-hour light/dark cycle with food and water ad libitum. Spontaneously breathing mice were subjected to closed head injury or sham surgery.

Mice were subjected to a single impact closed head injury (CHI; traumatic brain injury in mice) induced by 39 grams falling weight. As described in Kahriman et al. 2021 (38), animals were anesthetized with isoflurane (5% for induction, 2% for surgery, 1.5% for maintenance) in room air. Anesthesia was discontinued immediately prior to CHI and sham injury. Body temperature was monitored continuously with a rectal probe and maintained at 37.0±0.5° C. To alleviate pain, animals received 0.05 mg/kg subcutaneous buprenorphine (Med-Vet International, Mettawa, IL, USA) 30 minutes before anesthesia and every 6 hours afterwards for 24 hours. Additionally, each animal received 5 mg/kg subcutaneous carprofen (Patterson Veterinary, Devens, MA, USA) at the end of the anesthesia. CHI was produced using a weight drop device as previously described in detail (32, 34). Briefly, a 39 gram weight was freely dropped 15 cm to strike a cylindrical polyacetal transducer rod (DELRIN®, tip-diameter 2 mm, 17.4 g) that was placed with its tip directly on the exposed skull (target 2.5 mm posterior and 2.5 mm lateral from Bregma). Following CHI, the wound closed with interrupted sutures. Sham animals were anesthetized, surgically prepared (including skin incision), and placed under the impact device with the impactor touching the skull, but were not subjected to head impact.

CHI mice were then either not injected (“sham”) or received a volume flush (“1×”, 10 μL of isotonic aCSF) or a hypertonic flush (“2×”, 10 μL of 2× hypertonic aCSF). Levels of circulating (peripheral) inflammatory cytokines granulocyte colony-stimulating factor (G-CSF) and C—X—C motif chemokine ligand 1 (CXCL1) were measured in the plasma following treatments.

### Results

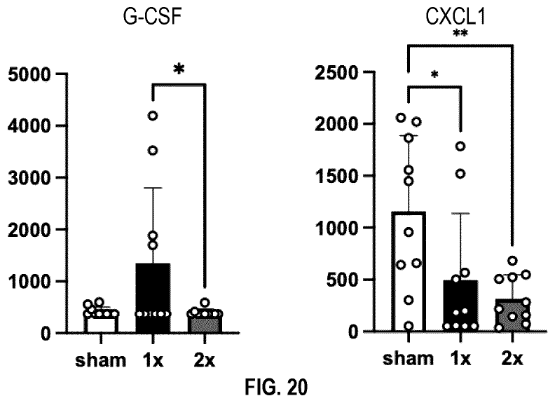

Cytokine levels in plasma decreased following treatments. G-CSF decreased in the 2× aCSF condition compared to the 1× aCSF condition (FIG. 20). CXCL1 decreased in the 2× aCSF and 1× aCSF conditions compared to sham (FIG. 20). These results suggest that CSF flow enhancement (CFE) may reduce neuroinflammation in the injured mouse brain.

### CFE can Improve Nanoparticle Delivery to the Injured Mouse Brain.

### Methods

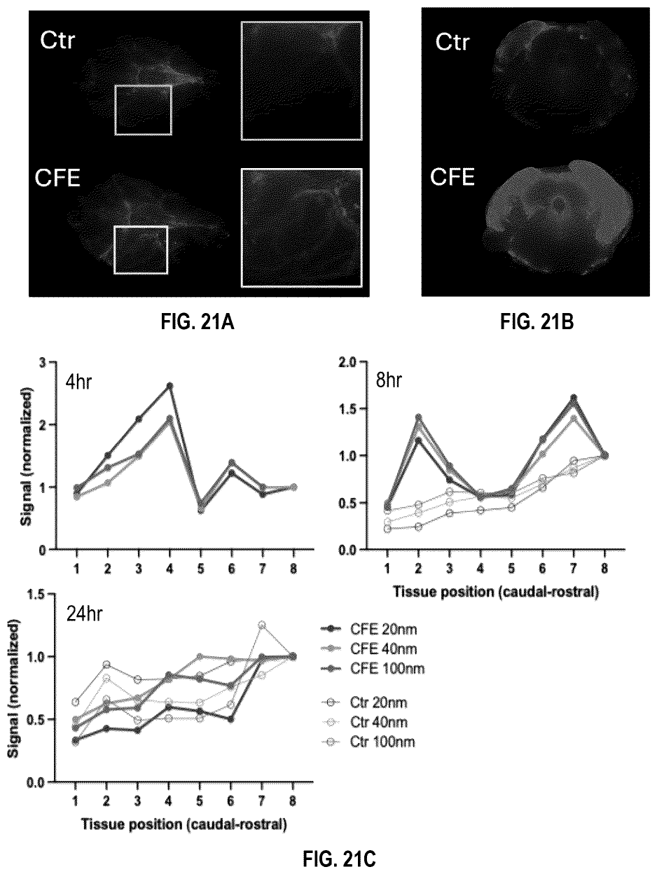

CHI mice (induced by 39 grams falling weight) received fluorescent polystyrene nanoparticle (FLUOSPHERES®; FS) infusions (20, 40, and 100 nm nanoparticle diameters) via intrathecal, cisterna magna administration site (IT-CM) at 4, 8, or 24 hours after CHI, with CFE (2× aCSF) or without CFE (1× aCSF) as a control. The ventral surface of whole brains was subjected to stereoscopic imaging (FIG. 21A; 8 hours after CHI), brains were sliced to 2 mm coronal sections and imaged with a stereoscope (FIG. 21B; 8 hours after CHI), and total fluorescence in each slice was quantified (FIG. 21C; 4, 8, and 24 hours after CHI).

### Results

Stereoscopic imaging of the ventral surface of whole brains revealed nanoparticle delivery to meningeal vasculature that is markedly increased after CFE (FIG. 21A; insets denote appearance of vascular trees). Quantifying total fluorescence in each slice revealed that CFE enhanced distribution when nanoparticles were administered IT-CM 8 hours after injury (FIG. 21C). The data show that administration of nanoparticles under CFE conditions 8 hours after CHI can be advantageous when compared to a similar administration made 24 hours post-injury. These results suggest that CFE improves nanoparticle delivery to the injured mouse brain.

### CFE can Enable Preferential Delivery to the Injured Hemisphere.

### Methods

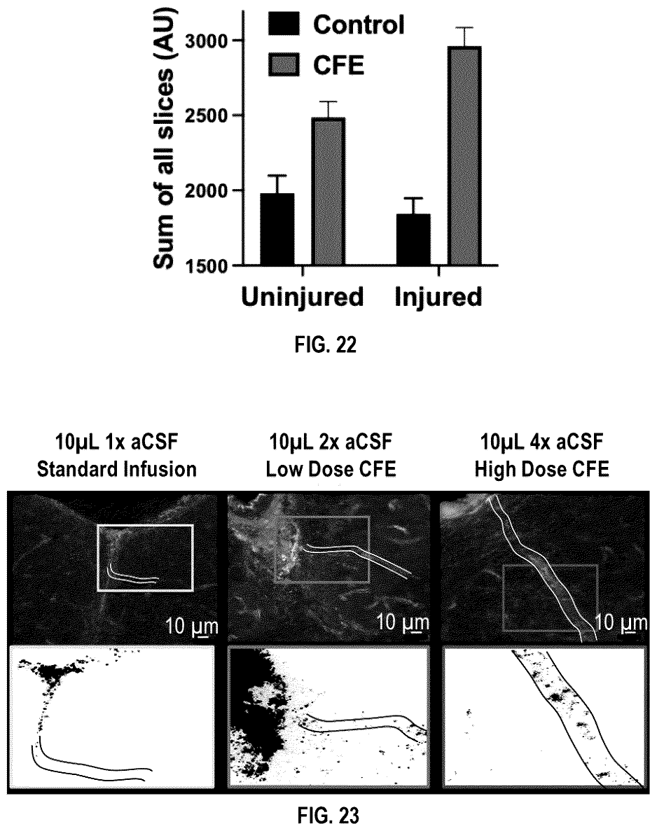

CHI mice (induced by 39 grams falling weight) received polystyrene nanoparticle (FLUOSPHERES®; FS) infusions (20, 40, and 100 nm nanoparticle diameters; data aggregated) via intrathecal, cisterna magna administration site administration (IT-CM) at 8 hours after CHI, with CFE (2× aCSF) or without CFE (1× aCSF) as a control. The fluorescent signal in the injured and uninjured hemispheres of each CHI mouse was quantified (FIG. 22).

### Results

The results show that CFE conditions (2× aCSF) leads to increased nanoparticle delivery to the injured hemisphere of the brain compared to the uninjured hemisphere. In contrast, control conditions (1× aCSF) show a similar distribution of nanoparticles between the injured and uninjured hemispheres of the brain. These results show that CFE conditions can enable preferential delivery of nanoparticles, and potentially of other particles in an infusion solution, to the injured hemisphere.

### CFE can Drive Perivascular and Parenchymal Delivery of Nanoparticles.

### Methods

Healthy mice were administered 100 nm polystyrene nanoparticles (FLUOSPHERES®; FS) into via intrathecal, cisterna magna administration site administration (IT-CM) 2 hours after CHI induced by 39 grams falling weight. Infusions were administered as 10 μL of standard (1× aCSF control), low dose CFE (2× aCSF), or high dose CFE (4× aCFE) infusion solutions. Two hours after administration of the infusion solutions, animals were sacrificed. Brain tissue was stained with a nuclear stain (DA-PI) and for the presence of aquaporin-4 (green). Aquaporin 4 labels astrocytic end feet and indirectly delineates vascular structures as astrocytic end feet wrap around blood vessels; dashed lines mark the paths of blood vessels and were applied to inverted images of the red channel, showing the signal of nanoparticles relative to the blood vessels.

### Results

Under control conditions with the standard (1× aCSF) infusion, nanoparticles congregated on the surface of brain but did not penetrate into parenchyma (e.g., brain tissue without blood vessels); no nanoparticle signal was detected beyond the blood vessel (FIG. 23, left panel). Low dose CFE conditions led to substantially greater signal of nanoparticles detected in the perivascular space, suggesting that the nanoparticles were being pushed into the tissue. Nanoparticle signal was reliably detected beyond the blood vessel, showing that nanoparticles were consistently present in brain parenchyma (FIG. 23, center panel). With increased strength of CFE conditions (high dose, 4× aCSF), occasional nanoparticles were observed outside of the blood vessel; however, the majority of nanoparticles were contained in within the blood vessel region (FIG. 23, right panel). High dose CFE may be associated with a slight increase in the size of blood vessels downstream of the administration site, potentially indicating an expansion of the perivascular space. Overall, these results show that strengthening CFE conditions (e.g., by using 2× aCSF or 4×CSF) drives nanoparticles down the perivascular space, with the 2× condition additionally enabling parenchymal entry. The ability to “tune” the distribution of nanoparticles and other functional agents in an infusion solution by changing the CFE strength (e.g., osmolarity or hypertonicity) may be useful in facilitating delivery to specific cell populations in the perivascular space.

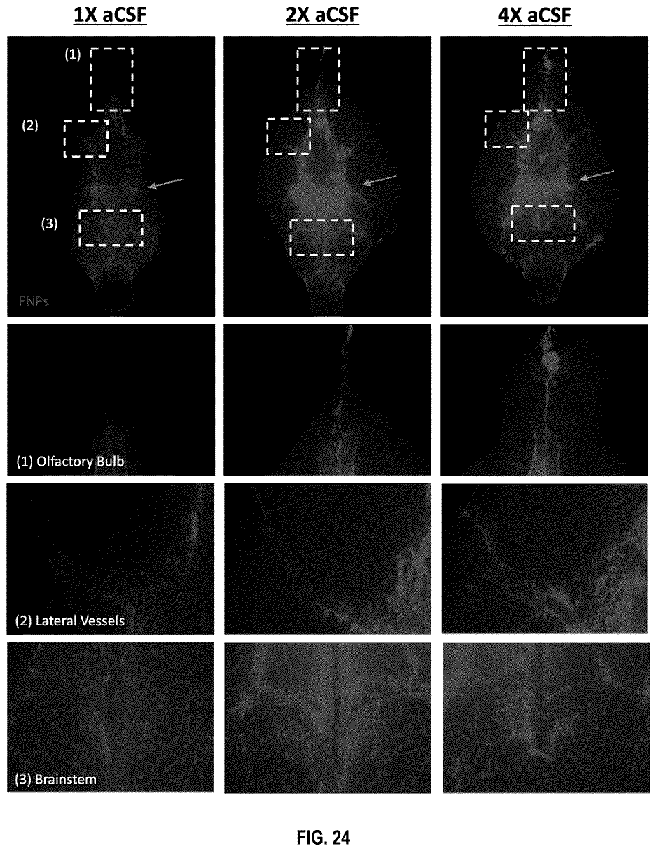

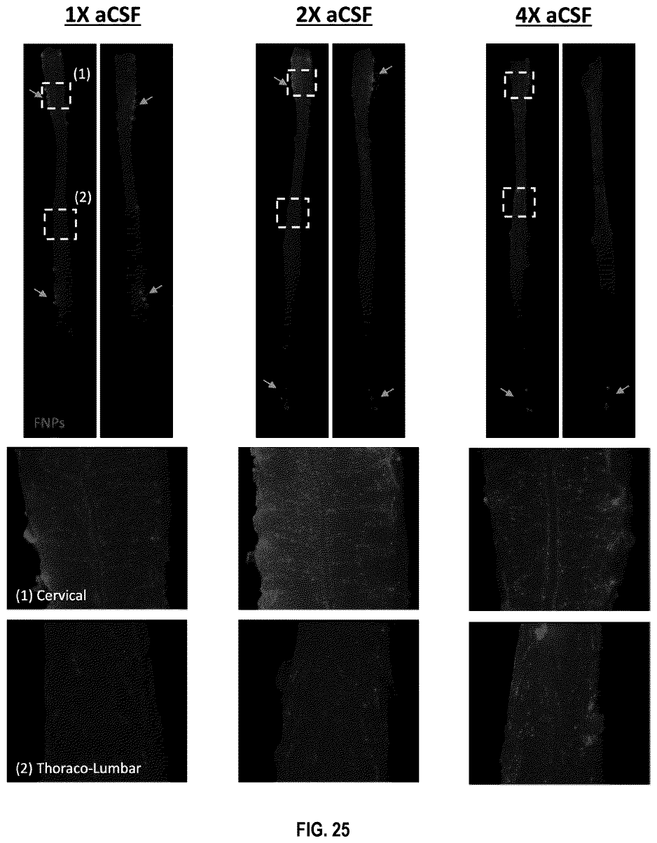

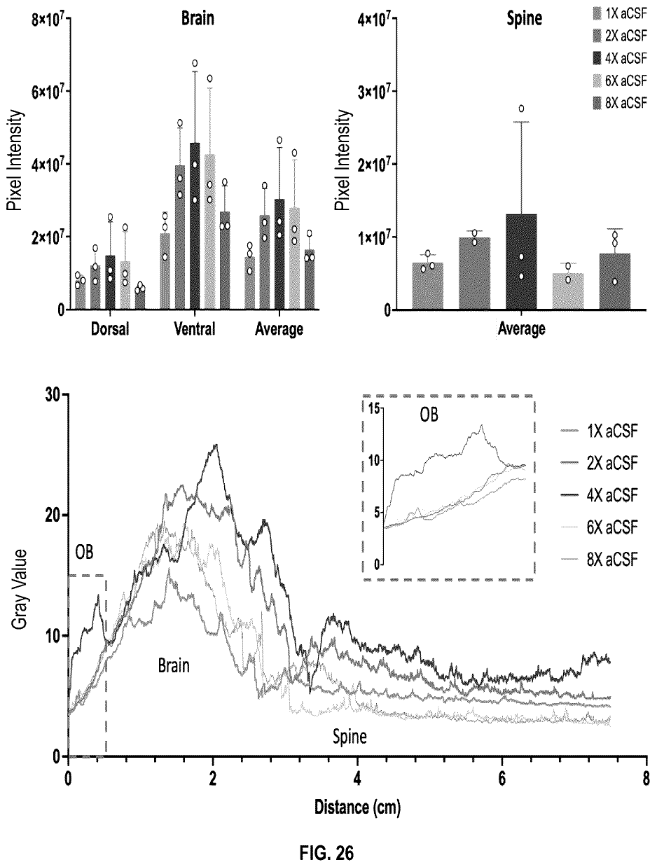

Imaging of the ventral surface (underside) of the brain show that when nanoparticles are administered, they localize with the meningeal vasculature in the Circle of Willis (an anatomical region that serves as an orienting landmark; not necessarily medically significant). With low dose CFE (2× aCSF) administration, there is substantially increased delivery to the olfactory bulb, lateral vessels, and brainstem. With a further increase to high dose CFE (4× aCSF), the effect appears to lessen. These results suggest that low dose CFE (2× aCSF) may be more optimal for delivery to the Circle of Willis when compared to high dose CFE (4× aCSF). Beyond the Circle of Willis, vascular trees branch and climb up the lateral sides of the brain. When CFE is administered (2× or 4× aCSF), vascular tress that are not visible under standard conditions (1× aCSF) show fluorescent nanoparticle signal. Similar results were observed in the spinal cord, with imaging showing evidence that nanoparticles localize with exiting nerve groups (FIG. 25, top panels; see arrows). This localization pattern could potentially be of use, for example, in analgesic delivery to inflamed nerve roots. Quantification of data, including the results represented in FIGS. 24-25, show that 2× and 4× aCSF can be beneficial in terms of colloid distribution throughout the central nervous system (FIG. 26). Higher intensity CFE conditions utilizing 6× or 8× aCSF may pose tolerability challenges.

Overall, these results suggest that under CFE conditions, nanoparticles are travelling much further than under standard infusion conditions.

### CFE Enhances Nanoparticle Delivery to Leptomeningeal Metastases.

### Methods

### Cell Culture

mCherry and luciferase transduced group 3 medulloblastoma cells (HDMB03-mCherry/Luc) were maintained in Roswell Park Memorial Institute (RPMI) 1640 cell culture medium (RPMI1640) with 10% fetal bovine serum (FBS), 1% penicillin-streptomycin, and 1% essential amino acids. Cells were incubated at 37° C. with 5% CO₂.

### Tumor Induction, Monitoring, and Tissue Processing

All experiments, procedures and animal care practices were approved by the UMass Chan Medical School's Institutional Animal Care and Use Committee and were performed in accordance with all relevant guidelines. Healthy, female NOD scid gamma (NSG) albino mice with age of within 6 to 8 weeks old were used for in vivo experiments.

The injection procedure was modified from a report published previously by Liu et al. (Direct CSF injection of MnCl(2) for dynamic manganese-enhanced MRI. Magn Reson Med. 2004 May; 51(5):978-87. doi: 10.1002/mrm.20047. PMID: 15122680). Briefly, on the day of the procedure, animals were weighted prior to the injection. Mice were then anesthetized under isoflurane and positioned on the bench with the superior aspect of the neck hyperextended to expose the back of the neck. The fur at the top of the head and back of the neck was then shaved to have their cisterna magna area exposed. The injection process was carried out with a puncture made by a 10 μL Hamilton neuro-syringe with 33-gauge needle between the skull and the C1 vertebrae. Three millimeters of the needle was inserted into the cisterna magna in order to avoid damaging the cerebellum or brain stem. The standard infusion of tumor cell-containing media was done throughout an approximate 60 to 70 second duration and the needle was kept in place for an additional 5 to 10 seconds before it was withdrawn. The mice were then monitored to see if there were any neurological symptoms or mobility disfunction. Only the mice with normal behaviors after the infusion were included in the study. Later, all tumor bearing mice, plus healthy control mice, were monitored and weighed daily.

The growth of the tumor was monitored by In Vivo Imaging System (IVIS) twice per week. Briefly, D-luciferin solution with a concentration of 150 mg/kg was injected into the mice via intraperitoneal (IP) Injection. Luminescence intensity was captured by IVIS Spectrum CT (Perkin-Elmer, Waltham, MA) 20 minutes after luciferase injection (data not shown).

### Processing and Imaging

For brain and spinal cord with axial skeleton, briefly, the skin, organs, and muscles were removed for fluorescence imaging. The whole brain and spinal cord fluorescent tile images were taken using a Leica stereoscope (Leica M205 FA, Wetzlar, Germany).

### Results

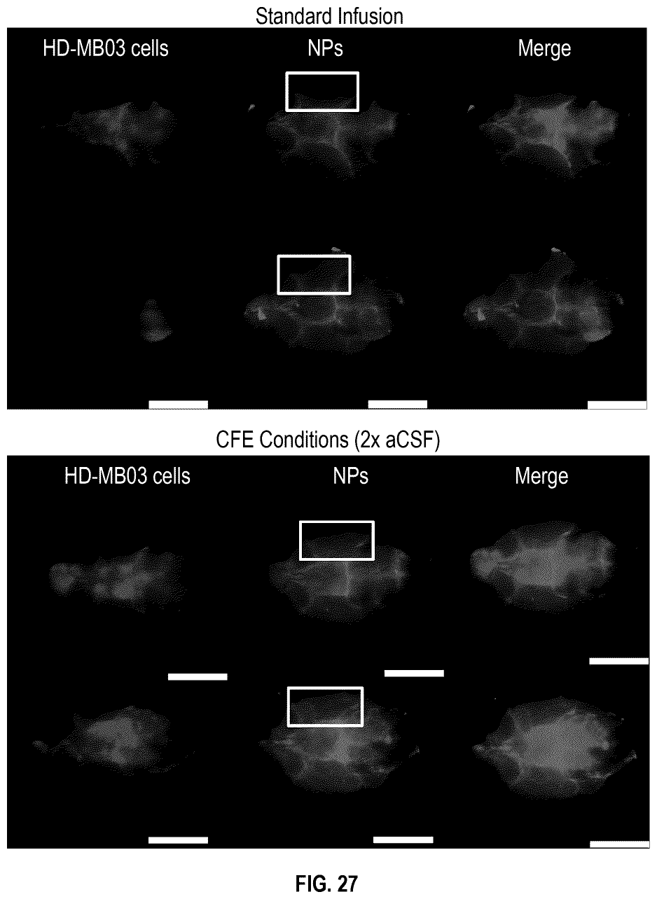

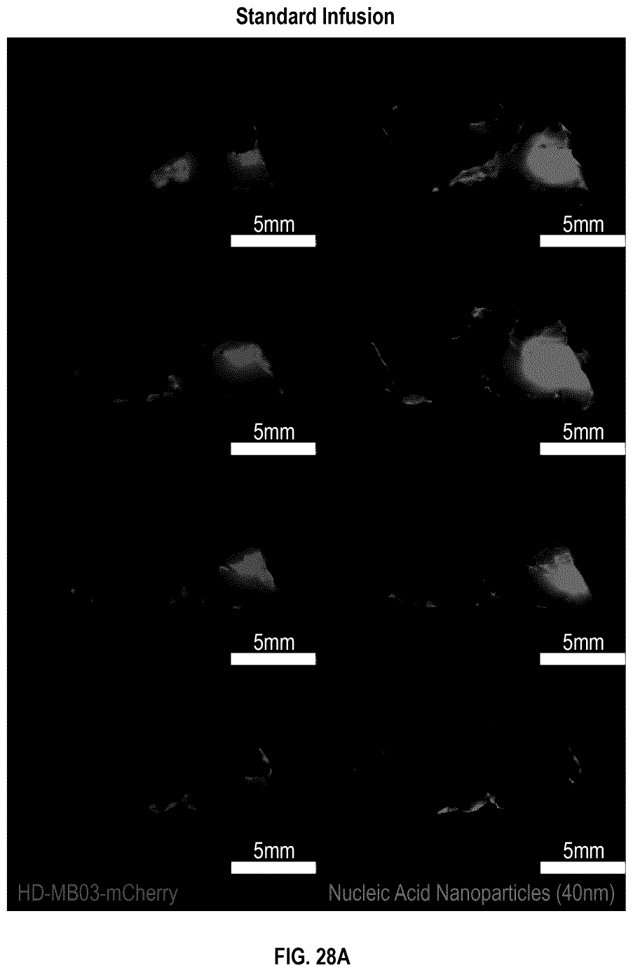

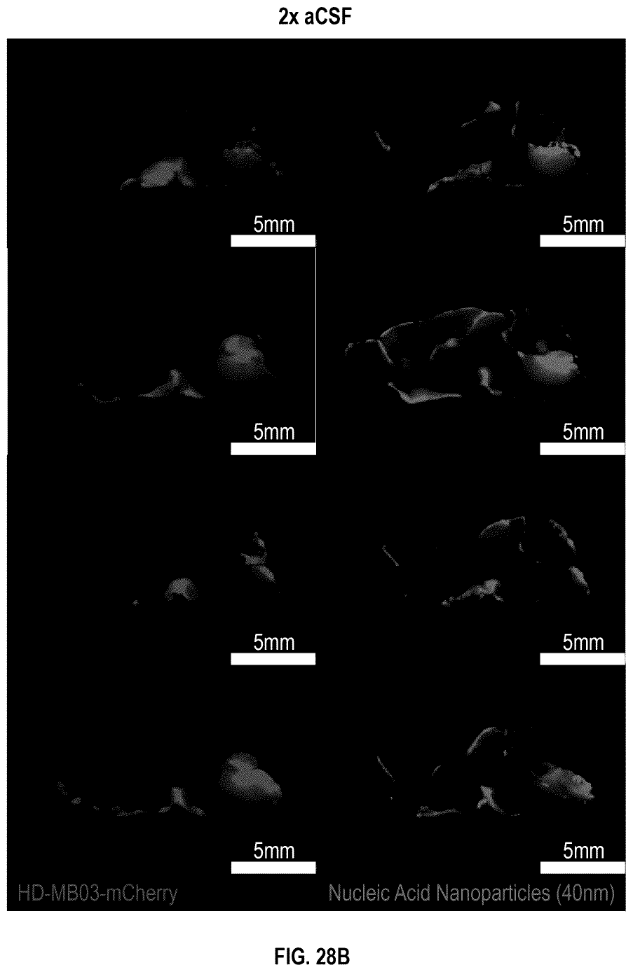

The results show that CFE enhances nanoparticle delivery to leptomeningeal metastases (LM; tumor cells on the surfaces of the brain and spinal cord) (FIG. 27), and that CFE redirects nucleic acid nanoparticle distribution from the injection site to sites of tumor metastasis under CFE conditions (FIGS. 28A-28B) in brains from NSG mice bearing orthotopic medulloblastoma (MB; HD-MB03 cells) exhibiting LM.

Negatively Charged Nanoparticles Exhibit Smooth, Unidirectional Flow within the Subarachnoid Space.

### Methods

Mice received a cranial window for intravital microscopy. Polystyrene nanoparticles were administered via the cisterna magna at a concentration of 20 mg/ml. Real-time intravital imaging demonstrated distinct nanoparticle properties and movement within the subarachnoid space. The subarachnoid space was identified by its borders, where substance movement and signal abruptly drops off.

### Results

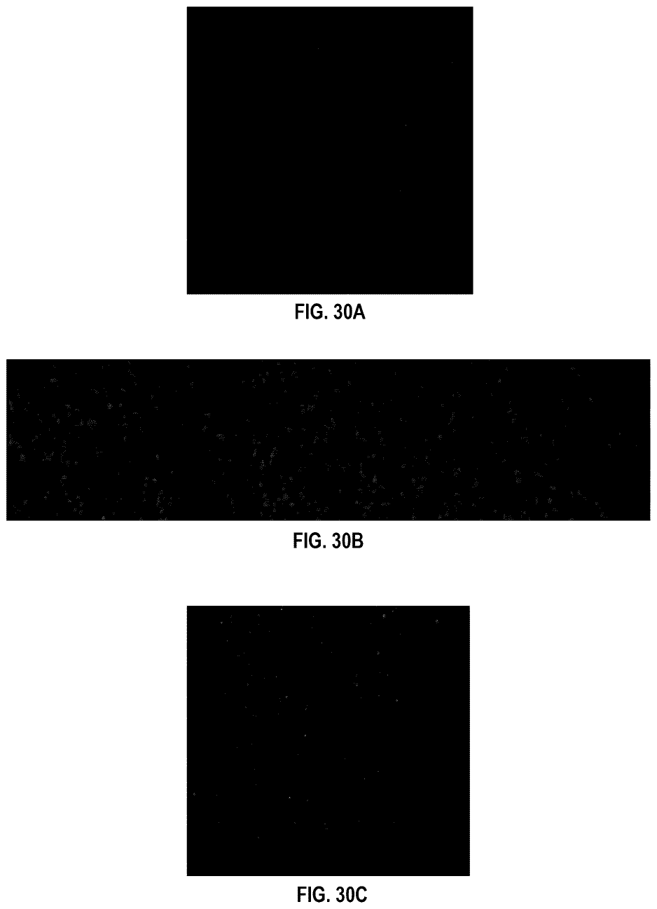

Non-PEGylated (standard) nanoparticles present as a distinctly identifiable colloids, with spherical shape and uniform size (FIG. 30A). This is consistent with the expectation that highly negatively charged nanoparticles will repel one another in solution. Dynamic imaging shows smooth, unidirectional flow. In contrast, PEGylated nanoparticles present as non-uniformly distributed aggregates (FIG. 30B). Dynamic imaging shows entrainment of nanoparticles to each other. Nanoparticle motion is characteristically oscillatory, with this feature of movement corresponding to the oscillatory motion of CSF due to respiration and cardiac rhythms. The net directional movement of highly PEGylated nanoparticles is restricted. PEGylated nanoparticles do not disperse or mix readily within the subarachnoid space. When the concentration of PEGylated nanoparticles is dropped from 20 mg/ml to 5 mg/ml, aggregation of nanoparticles is reduced and flow becomes directional again (FIG. 30C). These data support the hypothesis that PEGylation leads to nanoparticle entrainment within the confined and low-protein microenvironment of the subarachnoid space.

### REFERENCES

(1) Withrow D R, Berrington de Gonzalez A, Lam C J K, Warren K E, Shiels M S. Trends in pediatric central nervous system tumor incidence in the United States, 1998-2013. Cancer Epidemiol Biomarkers Prev. 2019; 28:522-530.
(2) Peterson K, Turos-Cabal M, Salvador A D, Palomo I, Howell A J, Vieira M E, Greiner S M, Barnoud T, Rodriguez-Blanco J. Mechanistic insights into medulloblastoma relapse. Pharmacol. Ther. 2024 108637
(3) Cocito C, Martin B, Giantini-Larsen A, Valcarce-Aspegren M, Souweidane M M, Szalontay L, Dahmane N, Greenfield J P. Leptomeningeal dissemination in pediatric brain tumors. Neoplasia 2023 39:100898
(4) Householder K T, Dharmaraj S, Sandberg D I, Wechsler-Reya R J, Sirianni R W. Fate of nanoparticles in the central nervous system after intrathecal injection in healthy mice. Scientific Reports 2019 9(1):12587
(5) Fowler M J, Cotter J D, Knight B E, Sevick-Muraca E M, Sandberg D I, Sirianni R W. Intrathecal drug delivery in the era of nanomedicine. Adv Drug Deliv Rev 2020:165-166:77-95
(6) Mitchell M J, Billingsley M M, Haley R M, Wechsler M E, Peppas N A, Langer R. Engineering precision nanoparticles for drug delivery. Nat Rev Drug Disc 2021 20:101-124
(7) Toy R, Peiris P M, Ghaghada K B, Karathanasis E. Shaping cancer nanomedicine: The effect ofparticle shape on the in vivo journal of nanoparticles. Nanomedicine (2014) 9(1):121-134
(8) McKee A C, Robinson M E. Military-related traumatic brain injury and neurodegeneration. Alzheimers Dement. June 2014; 10(3 Suppl):S242-53.
(9) Le T D, Gurney J M, Singh K P, et al. Trends in Traumatic Brain Injury Among U.S. Service Members, 2002-2016. Am J Prev Med. 2023; 65(2)
(10) Yaffe K, Lwi S J, Hoang T D, et al. Military-related risk factors in female veterans and risk of dementia. Neurology. 2019; 92(3):e205-e211.
(11) Snyder H M, Carare R O, DeKosky S T, et al. Military-related risk factors for dementia. Alzheimers Dement. 2018; 14(12):1651-1662.
(12) Crane P K, Gibbons L E, Dams-O'Connor K, et al. Association of TBI With Late-Life Neurodegenerative Conditions. JAMA Neurol. 2016; 73(9):1062-9.
(13) LoBue C, Denney D, Hynan L S, et al. Self-Reported Traumatic Brain Injury and Mild Cognitive Impairment: Increased Risk and Earlier Age of Diagnosis. J Alzheimers Dis. 2016; 51(3):727-36.
(14) LoBue C, Woon F L, Rossetti H C, Hynan L S, Hart J, Cullum C M. Traumatic brain injury history and progression from mild cognitive impairment to Alzheimer disease. Neuropsychology. May 2018; 32(4):401-409.
(15) Gardner R C, Burke J F, Nettiksimmons J, Kaup A, Barnes D E, Yaffe K. Dementia risk after traumatic brain injury vs nonbrain trauma: the role of age and severity. JAMA Neurol. December 2014; 71(12):1490-7.
(16) Nordstrom P, Michaelsson K, Gustafson Y, Nordstrom A. Traumatic brain injury and young onset dementia: a nationwide cohort study. Ann Neurol. March 2014; 75(3):374-81
(17) Guo Z, Cupples L A, Kurz A, et al. Head injury and the risk of AD in the MIRAGE study. Neurology. 2000; 54(6):1316-23.
(18) Chen H, Richard M, Sandler D P, Umbach D M, Kamel F. Head injury and amyotrophic lateral sclerosis. Am J Epidemiol. 2007; 166(7):810-6
(19) Pupillo E, Poloni M, Bianchi E, et al. Trauma and amyotrophic lateral sclerosis: a european population-based case-control study from the EURALS consortium. Amyotroph Lateral Scler Frontotemporal Degener. February 2018; 19(1-2):118-125.
(20) Delic V, Beck K D, Pang K C H, Citron B A. Biological links between TBI and Parkinson's. Acta Neuropathol Commun. Apr. 7, 2020; 8(1):45.
(21) Lu Y, Jarrahi A, Moore N, et al. Inflammaging, cellular senescence, and cognitive aging after TBI. Neurobiol Dis. May 2023; 180:106090
(22) Barker S, Paul B D, Pieper A A. Increased Risk of Neurodegenerative Disease after Traumatic Brain Injury. Biomedicines. 2023; 11(4)
(23) Hussain R, Tithof J, Wang W, et al. Potentiating glymphatic drainage minimizes post-traumatic cerebral oedema. Nature. 2023; 623(7989):992-1000.
(24) Kanda K, Adachi O, Kawatsu S, et al. Oxygenation of the cerebrospinal fluid with artificial cerebrospinal fluid can ameliorate a spinal cord ischemic injury in a rabbit model. J Thorac Cardiovasc Surg. November 2016; 152(5):1401-1409.
(25) Valitsky M, Benhamron S, Nitzan K, et al. Cerebrospinal Fluid (CSF) Exchange with Artificial CSF Enriched with Mesenchymal Stem Cell Secretions Ameliorates Experimental Autoimmune Encephalomyelitis. Int J Mol Sci. Apr. 11, 2019; 20(7)
(26) Venkatraman V, Filiano A J, Xu L, et al. Filtered Cerebrospinal Fluid From Patients With Amyotrophic Lateral Sclerosis Displays an Altered Proteome and Affects Motor Phenotype in a Mouse Model. Cureus. December 2022; 14(12):e32980.
(27) Benhamron S, Nitzan K, Valitsky M, et al. Cerebrospinal Fluid (CSF) Exchange Therapy with Artificial CSF Enriched with Mesenchymal Stem Cell Secretions Ameliorates Cognitive Deficits and Brain Pathology in Alzheimer's Disease Mice. J Alzheimers Dis. 2020; 76(1):369-385.
(28) Hablitz L M, Nedergaard M. The Glymphatic System: A Novel Component of Fundamental Neurobiology. J Neurosci. Sep. 15, 2021; 41(37):7698
(29) Reiter R J, Sharma R, et al. Brain washing and neural health: role of age, sleep, and the CSF melatonin rhythm. Cell Mol Life Sci. 2023; 80(4):88.
(30) Peters M E, Lyketsos C G. The glymphatic system's role in traumatic brain injury-related neurodegeneration. Mol Psychiatry. July 2023; 28(7):2707-2715
(31) Marchesini N, Fernandez Londono L L, Boaro A, et al. Hyperosmolar therapies for neurological deterioration in mild and moderate traumatic brain injury: A scoping review. Brain Inj. Jul. 29, 2023; 37(9):1107-1115.
(32) Bouley J, Chung D Y, Ayata C, Brown R H, et al. Cortical Spreading Depression Denotes Concussion Injury. J Neurotrauma. 2019; 36(7):1008-1017.
(33) Kahriman A, Bouley J, Tuncali I, et al. Repeated mild traumatic brain injury triggers pathology in asymptomatic C90RF72 transgenic mice. Brain. Dec. 1, 2023; 146(12):5139-5152.
(34) Henninger N, Bouley J, Sikoglu E M, et al. Attenuated traumatic axonal injury and improved functional outcome after traumatic brain injury in mice lacking Sarm1. Brain. April 2016; 139(Pt 4):1094-105.
(35) Henninger N, Dutzmann S, Sicard K M, Kollmar R, Bardutzky J, Schwab S. Impaired spatial learning in a novel rat model of mild cerebral concussion injury. Exp Neurol. October 2005; 195(2):447-57.
(36) Henninger N, Sicard K M, Li Z, et al. Differential recovery of behavioral status and brain function assessed with functional magnetic resonance imaging after mild traumatic brain injury in the rat. Crit Care Med. November 2007; 35(11):2607-14.
(37) Bouley J, Henninger N. Lateral Ventricle Attenuates Traumatic Axonal Injury after Closed Head Injury. J Neurotrauma. 2017; 34(13):2190-2191.
(38) Kahriman A, Bouley J, Smith T W, Bosco D A, Woerman A L, Henninger N. Mouse closed head traumatic brain injury replicates the histological tau pathology pattern of human disease: characterization of a novel model and systematic review of the literature. Acta Neuropathol Commun. 2021; 9(1):118.
(39) Zhong J, Gunner G, Henninger N, Schafer D P, Bosco D A. Intravital Imaging of Fluorescent Protein Expression in Mice with a Closed-Skull Traumatic Brain Injury and Cranial Window Using a Two-Photon Microscope. J Vis Exp. Apr. 21, 2023; (194)
(40) Gardner R C, Bahorik A, Kornblith E S, Allen I E, Plassman B L, Yaffe K. Systematic Review, Meta-Analysis, and Population Attributable Risk of Dementia Associated with Traumatic Brain Injury in Civilians and Veterans. J Neurotrauma. April 2023; 40(7-8):620-634.
(41) Henninger N, Izzy S, Carandang R, et al. Severe leukoaraiosis portends a poor outcome after TBI. Neurocrit Care. 2014; 21(3):483-95.
(42) Henninger N, Compton R A, Khan M W, Carandang R, Hall W, Muehlschlegel S. “Don't lose hope early”: Hemorrhagic diffuse axonal injury on head CT is not associated with poor outcome in moderate to severe traumatic brain injury patients. J Trauma Acute Care Surg. March 2018; 84(3):473-482.
(43) Sirianni R W, Kremer J, Guler I, Chen Y L, Keeley F W, Saltzman W M. Effect of extracellular matrix elements on the transport of paclitaxel through an arterial wall tissue mimic. Biomacromolecules. 2008; 9(10):2792-8.
(44) Sirianni R W, Zheng M Q, Saltzman W M, Huang Y, Carson R E. Direct, quantitative, and noninvasive imaging of the transport of active agents through intact brain with positron emission tomography. Mol Imaging Biol. October 2013; 15(5):596-605.
(45) Sirianni R W, Zheng M Q, Patel T R, et al. Radiolabeling of poly(lactic-co-glycolic acid) (PLGA) nanoparticles with biotinylated F-18 prosthetic groups and imaging of their delivery to the brain with positron emission tomography. Bioconjug Chem. Dec. 17, 2014; 25(12):2157-65.
(46) Zhou J, Patel T R, Sirianni R W, et al. Highly penetrative, drug-loaded nanocarriers improve treatment of GBM. Proc Natl Acad Sci 2013; 110(29):1175
(47) Medina D X, Chung E P, Teague C D, Bowser R, Sirianni R W. Intravenously Administered, Retinoid Activating Nanoparticles Increase Lifespan and Reduce Neurodegeneration in the SOD1(G93A) Mouse Model of ALS. Front Bioeng Biotechnol. 2020; 8:224.
(48) Householder K T, DiPerna D M, Chung E P, et al. pH driven precipitation of quisinostat onto PLA-PEG nanoparticles enables treatment of intracranial glioblastoma. Colloids Surf B Biointerfaces. 2018; 166:37-44.
(49) Cook R L, Householder K T, Chung E P, Prakapenka A V, DiPerna D M, Sirianni R W. A critical evaluation of drug delivery from ligand modified nanoparticles: Confounding small molecule distribution and efficacy in the central nervous system. J Control Release. Dec. 28, 2015; 220(Pt A):89-97.
(50) Householder K T, DiPerna D M, Chung E P, et al. Intravenous delivery of camptothecin-loaded PLGA nanoparticles for the treatment of intracranial glioma. Int J Pharm. 20 2015; 479(2):374-80.
(51) Prakapenka A V, Quihuis A M, Carson C G, Patel S, Bimonte-Nelson H A, Sirianni R W. Poly(lactic-co-glycolic Acid) Nanoparticle Encapsulated 170-Estradiol Improves Spatial Memory and Increases Uterine Stimulation in Middle-Aged Ovariectomized Rats. Front Behav Neurosci. 2020; 14:597690.
(52) Sandberg D I, Yu B, Patel R, et al. Infusion of 5-Azacytidine (5-AZA) into the fourth ventricle or resection cavity in children with recurrent posterior Fossa Ependymoma: a pilot clinical trial. J Neurooncol. January 2019; 141(2):449-457.
(53) Sandberg D I, Kharas N, Yu B, et al. High-dose MTX110 (soluble panobinostat) safely administered into the fourth ventricle in a nonhuman primate model. J Neurosurg Pediatr. May 1, 2020; 26(2):127-135.
(54) DeJesus-Hernandez, M., et al. Expanded GGGGCC hexanucleotide repeat in noncoding region of C90RF72 causes chromosome 9p-linked FTD and ALS. Neuron 72, 245-256 (2011).
(55) Renton, A. E., et al. A hexanucleotide repeat expansion in C90RF72 is the cause of chromosome 9p21-linked ALS-FTD. Neuron 72, 257-268 (2011).
(56) Peters, O. M., Ghasemi, M. & Brown, R. H., Jr. Emerging mechanisms of molecular pathology in ALS. J Clin Invest 125, 1767-1779 (2015).
(57) van Blitterswijk, M., et al. Association between repeat sizes and clinical and pathological characteristics in carriers of C90RF72 repeat expansions (Xpansize-72): a cross-sectional cohort study. Lancet neurology 12, 978-988 (2013).
(58) Cook, C. & Petrucelli, L. Genetic Convergence Brings Clarity to the Enigmatic Red Line in ALS. Neuron 101, 1057-1069 (2019).
(59) Woollacott, I. O. & Rohrer, J. D. The clinical spectrum of sporadic and familial forms of frontotemporal dementia. Journal of neurochemistry 138 Suppl 1, 6-31 (2016).
(60) Olney, N. T., Spina, S. & Miller, B. L. Frontotemporal Dementia. Neurol Clin 35, 339-374 (2017).
(61) Lehmer, C., et al. Poly-GP in cerebrospinal fluid links C9orf72-associated dipeptide repeat expression to the asymptomatic phase of ALS/FTD. EMBO Mol Med 9, 859-868 (2017).
(62) Graham, N. S. & Sharp, D. J. Understanding neurodegeneration after traumatic brain injury: from mechanisms to clinical trials in dementia. Journal of neurology, neurosurgery, and psychiatry 90, 1221-1233 (2019).
(63) LoBue, C., et al. Traumatic brain injury history is associated with earlier age of onset of frontotemporal dementia. Journal of neurology, neurosurgery, and psychiatry 87, 817-820 (2016).
(64) Wang, H. K., et al. Traumatic brain injury causes frontotemporal dementia and TDP-43 proteolysis. Neuroscience 300, 94-103 (2015).
(65) Kalkonde, Y. V., et al. Medical and environmental risk factors associated with frontotemporal dementia: a case control study in a veteran population. Alzheimers Dement 8, 204-210 (2012).
(66) Deutsch, M. B., Mendez, M. F. & Teng, E. Interactions between traumatic brain injury and frontotemporal degeneration. Dement Geriatr Cogn Disord 39, 143-153 (2015).
(67) Maas, A. I. R., et al. Traumatic brain injury: integrated approaches to improve prevention, clinical care, and research. Lancet neurology 16, 987-1048 (2017).
(68) Guskiewicz, K. M., et al. Association between recurrent concussion and late-life cognitive impairment in retired professional football players. Neurosurgery 57, 719-726; discussion 719-726 (2005).
(69) Liu, Y., et al. C9orf72 BAC mouse model with motor deficits and neurodegenerative features of ALS/FTD. Neuron 90, 521-534 (2016).
(70) Wang, J. T., Medress, Z. A. & Barres, B. A. Axon degeneration: molecular mechanisms of a self-destruction pathway. The Journal of cell biology 196, 7-18 (2012).
(71) Mackenzie, I. R., et al. Dipeptide repeat protein pathology in C90RF72 mutation cases: clinico-pathological correlations. Acta neuropathologica 126, 859-879 (2013).
(72) Iliff, J. J., et al. Impairment of glymphatic pathway function promotes tau pathology after traumatic brain injury. The Journal of neuroscience: the official journal of the Society for Neuroscience 34, 16180-16193 (2014).
(73) Plog, B. A., et al. Biomarkers of traumatic injury are transported from brain to blood via the glymphatic system. The Journal of neuroscience: the official journal of the Society for Neuroscience 35, 518-526 (2015).
(74) Louveau, A., et al. CNS lymphatic drainage and neuroinflammation are regulated by meningeal lymphatic vasculature. Nature neuroscience 21, 1380-1391 (2018).
(75) Iliff, J. J., et al. A paravascular pathway facilitates CSF flow through the brain parenchyma and the clearance of interstitial solutes, including amyloid beta. Sci Transl Med 4, 147ra111 (2012).
(76) Da Mesquita, S., et al. Functional aspects of meningeal lymphatics in ageing and Alzheimer's disease. Nature 560, 185-191 (2018).
(77) Wang, L., et al. Deep cervical lymph node ligation aggravates AD-like pathology of APP/PSI mice. Brain Pathol 29, 176-192 (2019).
(78) Zou, W., et al. Blocking meningeal lymphatic drainage aggravates Parkinson's disease-like pathology in mice overexpressing mutated alpha-synuclein. Transl Neurodegener 8, 7 (2019).
(79) Wollinsky, K. H., et al. Liquorpheresis (CSF-filtration): an effective treatment in acute and chronic severe autoimmune polyradiculoneuritis (Guillain-Barre syndrome). Eur Arch Psychiatry Clin Neurosci 241, 73-76 (1991).
(80) Mackenzie, I. R., Rademakers, R. & Neumann, M. TDP-43 and FUS in amyotrophic lateral sclerosis and frontotemporal dementia. Lancet neurology 9, 995-1007 (2010).
(81) Bright, F., et al. Neuroinflammation in frontotemporal dementia. Nat Rev Neurol 15, 540-555 (2019).
(82) Beers, D. R. & Appel, S. H. Immune dysregulation in amyotrophic lateral sclerosis: mechanisms and emerging therapies. Lancet neurology 18, 211-220 (2019).
(83) Shao, F., Wang, X., Wu, H., Wu, Q. & Zhang, J. Microglia and neuroinflammation: Crucial pathological mechanisms in traumatic brain injury-induced neurodegeneration. Front Aging Neurosci 14, 825086 (2022).
(84) Bieniek, K. F., et al. The second NINDS/NIBIB consensus meeting to define neuropathological criteria for the diagnosis of chronic traumatic encephalopathy. Journal of neuropathology and experimental neurology 80, 210-219 (2021).
(85) Dogan, E. O., et al. Genetic ablation of Sarm1 attenuates expression and mislocalization of phosphorylated TDP-43 after mouse repetitive traumatic brain injury. Acta Neuropathol Commun 11, 206 (2023).
(86) Fowler, M. J., et al. Engineering subarachnoid trabeculae with electrospun poly(caprolactone) (PCL) scaffolds to study leptomeningeal metastasis in medulloblastoma. Biomater Adv 155, 213646 (2023).
(87) Henninger, N., et al. Spatial learning induces predominant downregulation of cytosolic proteins in the rat hippocampus. Genes, brain, and behavior 6, 128-140 (2007).
(88) Ferrer, I., Andres-Benito, P., Carmona, M., Assialioui, A. & Povedano, M. TDP-43 Vasculopathy in the Spinal Cord in Sporadic Amyotrophic Lateral Sclerosis (sALS) and Frontal Cortex in sALS/FTLD-TDP. Journal of neuropathology and experimental neurology 80, 229-239 (2021).
(89) Zhao, W., et al. TDP-43 activates microglia through NF-kappaB and NLRP3 inflammasome. Exp Neurol 273, 24-35 (2015).
(90) Swanson, K. V., Deng, M. & Ting, J. P. The NLRP3 inflammasome: molecular activation and regulation to therapeutics. Nature reviews. Immunology 19, 477-489 (2019).
(91) Mendiola, A. S. & Cardona, A. E. The IL-1beta phenomena in neuroinflammatory diseases. J Neural Transm (Vienna) 125, 781-795 (2018).
(92) Thome, J. G., Reeder, E. L., Collins, S. M., Gopalan, P. & Robson, M. J. Contributions of Interleukin-1 Receptor Signaling in Traumatic Brain Injury. Front Behav Neurosci 13, 287 (2019).
(93) Medina D X, Householder K T, Ceton R, Kovalik T, Heffernan J, Shankar R V, Bowser R P, Wechsler-Reya R J, Sirianni R W. “Optical barcoding of PLGA for multispectral analysis of nanoparticle fate in vivo” Journal of Controlled Release. 10; 253:172-182 PMID: 28263836
(94) Sandberg, DI. (2021-). Combination Intraventricular Chemotherapy Pilot Study: 5-Azacytidine (5-AZA) and Trastuzumab Infusions Into the Fourth Ventricle or Resection Cavity in Children and Adults With Recurrent or Residual Posterior Fossa Ependymoma. Identifier NCT04958486. https://clinicaltrials.gov/study/NCT04958486
(95) Sandberg, DI. (2020-). Infusion of Panobinostat (MTX110) Into the Fourth Ventricle in Children and Adults With Recurrent Medulloblastoma. Identifier NCT04315064. https://clinicaltrials.gov/study/NCT04315064
(96) Heffernan J M, Overstreet D J, Le L D, Vernon B L, Sirianni R W. (2014) “Bioengineered hydrogels for 3D analysis of glioblastoma invasion and proliferation”. Annals of Biomedical Engineering, epub first. DOI: 10.1007/s10439-014-1223-1. PMID: 25515315
(97) Heffernan J M, Overstreet D J, Srinivasan S, Le L D, Vernon B L, Sirianni R W. “Temperature responsive hydrogels enable transient three-dimensional tumor cultures via rapid cell recovery” Journal of Biomaterials Research: Part A 104(1):17-25. PMID: 26123863
(98) Heffernan J M, McNamara J B, Borwege S, Vernon B L, Sanai N, Mehta S, Sirianni R W. (2017) “PNIPAAm-co-Jeffamine® (PNJ) scaffolds as in vitro models for niche enrichment of glioblastoma stemlike cells” Biomaterials 143:149-58 PMC5605153
(99) McCall R L, Sirianni R W. (2013) PLGA nanoparticles formed by single- or double-emulsion with vitamin E-TPGS. Journal of Visualized Experiments. (82):51015. PMID: 24429733
(100) Kobes J E, Daryaei I, Howison C M, Bontrager J G, Sirianni R W, Meuillet E J, Pagel M D. (2016) “Improved treatment of pancreatic cancer with drug delivery nanoparticles loaded with a novel AKT/PDK1 inhibitor” Pancreas 45(9):1158-66 PMID: 26918875
(101) Dutta D, Salifu M, Sirianni R W, Stabenfeldt S. (2016) “Tailoring sub-micron PLGA particle release profiles via centrifugal fractioning” Journal of Biomedical Materials Research: Part A 104(3):688-96. PMC4980271
(102) Chaudhuri S, Fowler M J, Baker C, Stopka S A, Regan M S, Sablatura L, Broughton C, Knight B E, Stabenfeldt S E, Agar NYR, Sirianni R W. (2021) “Beta-cyclodextrin-poly(beta-amino ester) nanoparticles are a generalizable strategy for high loading and sustained release of HDAC inhibitors” ACS Applied Materials and Interfaces 13(18) 20960-20973. PMC8153536
(103) Decker Y, Kramer J, Xin L, Müller A, Scheller A, Fassbender K, Prou1× ST. Magnetic resonance imaging of cerebrospinal fluid outflow after low-rate lateral ventricle infusion in mice. JCI Insight. 2022 Feb. 8; 7(3):e150881. doi: 10.1172/jci.insight.150881. PMID: 34905509; PMCID: PMC8855808.
(104) Hablitz L M, Nedergaard M. The glymphatic system. Curr Biol. 2021 Oct. 25; 31(20):R1371-R1375. doi: 10.1016/j.cub.2021.08.026. PMID: 34699796.
(105) Anonymous (1999) Consensus conference. Rehabilitation of persons with traumatic brain injury. NIH consensus development panel on rehabilitation of persons with traumatic brain injury. JAMA 282: 974-983
(106) Fleminger S, Ponsford J (2005) Long term outcome after traumatic brain injury. BMJ 331:1419-1420. https://doi. org/10. 1136/bmj. 331. 7530. 1419
(107) Peeters W, van den Brande R, Polinder S, Brazinova A, Steyerberg E W, Lingsma H F, Maas A I (2015) Epidemiology of traumatic brain injury in Europe. Acta Neurochir (Wien) 157:1683-1696. https://doi. org/10. 1007/s00701-015-2512-7
(108) Hyder A A, Wunderlich C A, Puvanachandra P, Gururaj G, Kobusingye O C (2007) The impact of traumatic brain injuries: a global perspective. NeuroRehabilitation 22:341-353
(109) Graham N S N, Cole J H, Bourke N J, Schott J M, Sharp D J. Distinct patterns of neurodegeneration after TBI and in Alzheimer's disease. Alzheimers Dement. 2023 July; 19(7):3065-3077. doi: 10.1002/alz.12934. Epub 2023 Jan. 25. PMID: 36696255; PMCID: PMC10955776.
(110) Ramos-Cejudo J, Wisniewski T, Marmar C, Zetterberg H, Blennow K, de Leon M J, Fossati S. Traumatic Brain Injury and Alzheimer's Disease: The Cerebrovascular Link. EBioMedicine. 2018 February; 28:21-30. doi: 10.1016/j.ebiom.2018.01.021. Epub 2018 Jan. 31. PMID: 29396300; PMCID: PMC5835563.
(111) Pan K, Bricker W P, Ratanalert S, Bathe M. Structure and conformational dynamics of scaffolded DNA origami nanoparticles. Nucleic Acids Res. 2017 Jun. 20; 45(11):6284-6298. doi: 10.1093/nar/gkx378. PMID: 28482032; PMCID: PMC5499760
(112) Kahriman A, Bouley J, Bosco D A, Shazeeb M S, Henninger N. Differential association of baseline body weight and body-weight loss with neurological deficits, histology, and death after repetitive closed head traumatic brain injury. Neurosci Lett. 2022 Feb. 6; 771:136430. doi: 10.1016/j.neulet.2021.136430. Epub 2021 Dec. 29. PMID: 34973374; PMCID: PMC8821174.
(113) Dogan E O, Simonini S R, Bouley J, Weiss A, Brown R H Jr, Henninger N. Genetic Ablation of Sarm1 Mitigates Disease Acceleration after Traumatic Brain Injury in the SOD1^{G93A} Transgenic Mouse Model of Amyotrophic Lateral Sclerosis. Ann Neurol. 2025 May; 97(5):963-975. doi: 10.1002/ana.27174. Epub 2025 Jan. 10. PMID: 39791335; PMCID: PMC12011539.

### CONCLUSION

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. (canceled)

2. A method for treating a subject in need thereof, said method comprising administering an infusion solution to a cerebrospinal fluid (CSF) of the subject, wherein the infusion solution comprises a hypertonic solution.

3-6. (canceled)

7. The method of claim 2, wherein the subject has a disease or injury of the central nervous system (CNS).

8. The method of claim 2, wherein the hypertonic solution has an osmolarity at least about 1.1 times (1.1×) up to about 8 times (8×) an osmolarity of the CSF of the subject.

9. (canceled)

10. The method of claim 2, wherein the hypertonic solution comprises one or more electrolytes selected from sodium (Na⁺), potassium (K⁺), Calcium (Ca²⁺), magnesium (Mg²⁺), chloride (Cl⁻), and biocarbonate (HCO₃⁻), wherein an osmolarity of each of the one or more electrolytes is higher than an osmolarity of said electrolytes in the CSF of the subject.

11. (canceled)

12. The method of claim 2, wherein the subject is a human and wherein the hypertonic solution has an osmolarity of
up to about 2240 mOsm.

13. (canceled)

14. The method of claim 2, wherein the hypertonic solution comprises:
a) about 100 to about 200 mOsm Na⁺,
b) about 2 to about 4 mOsm K⁺,
c) about 1 to about 2.5 mOsm Ca²⁺,
d) about 1 to about 3 mOsm Mg²⁺,
e) about 100 to about 200 mOsm Cl⁻,
f) about 18 to about 27 mOsm HCO₃⁻, or
g) any combination of the foregoing.

15.-20. (canceled)

21. The method of claim 2, wherein the infusion solution further comprises a therapeutic agent.

22. The method of claim 2, the method further comprising the step of administering a therapeutic agent to the subject before, during, or after administering the infusion solution to the subject.

23. The method of claim 21, wherein the therapeutic agent comprises a nanoparticle, DNA, RNA, protein, peptide, lipid, liposome, viral vector, small molecule, large molecule, aptamer, chemotherapeutic, or a combination thereof.

24.-32. (canceled)

33. The method of claim 21, wherein a dimension of the therapeutic agent is up to about 1 micron.

34.-38. (canceled)

39. The method of claim 2, wherein the infusion solution is administered intrathecally.

40.-42. (canceled)

43. The method of claim 2, wherein the infusion solution has a volume of up to about half of a volume of a CSF of the subject.

44. (canceled)

45. The method of claim 2, wherein the subject has, is suspected to have, or is at risk for having a stroke, neurodegenerative disease, neuroinflammation, cancer affecting a central nervous system (CNS), disease or infection of the CNS, cerebrovascular disease, or a combination thereof.

46. The method of claim 2, wherein the subject has or is suspected to have a traumatic brain injury (TBI), traumatic spinal cord injury, or both.

47.-48. (canceled)

49. The method of claim 45, wherein the cancer is primary, metastatic, associated with leptomeningeal metastases (LM), or a combination thereof.

50. The method of claim 45, wherein the cancer comprises brain cancer, a brain metastasis, or both.

51. (canceled)

52. The method of claim 45, wherein the disease of the CNS is a lysosomal storage disorder.

53. The method of claim 45, wherein the subject has, is suspected to have, or is at risk for having an abnormal deposit comprising a neurotoxin, an inflammatory cytokine, or both.

54.-58. (canceled)

59. An infusion solution for use in treating a subject in need thereof, comprising a hypertonic solution, wherein the hypertonic solution comprises:
a) a total osmolarity of about 1.1 times (1.1×) to about 8 times (8×) a total osmolarity of the CSF of the subject,
b) an osmolarity of Na⁺ about 1.1 times (1.1×) to about 8 times (8×) an osmolarity of Na⁺ in the CSF of the subject,
c) an osmolarity of K⁺ about 1.1 times (1.1×) to about 8 times (8×) an osmolarity of K⁺ in the CSF of the subject,
d) an osmolarity of Ca² about 1.1 times (1.1×) to about 8 times (8×) an osmolarity of Ca² in the CSF of the subject,
e) an osmolarity of Mg²⁺ about 1.1 times (1.1×) to about 8 times (8×) an osmolarity of Mg²⁺ in the CSF of the subject,
f) an osmolarity of Cl⁻ about 1.1 times (1.1×) to about 8 times (8×) an osmolarity of Cl⁻ in the CSF of the subject,
g) an osmolarity of HCO₃⁻ about 1.1 times (1.1×) to about 8 times (8×) an osmolarity of HCO₃⁻ in the CSF of the subject, or
h) any combination of the foregoing.

60.-62. (canceled)

63. The infusion solution of claim 59, further comprising a therapeutic agent.

64.-78. (canceled)


SEQUENCE LISTING (4 sequences)

SEQ ID NO: 1 - DNA, 23 bp, Homo sapiens
  source 1..23: mol_type = other DNA
tgataactggagtacatttcgcc

SEQ ID NO: 2 - DNA, 22 bp, Homo sapiens
  source 1..22: mol_type = other DNA
cggtcataatgggtgagagtct

SEQ ID NO: 3 - DNA, 20 bp, Homo sapiens
  source 1..20: mol_type = other DNA
tgacgtggacatccgcaaag

SEQ ID NO: 4 - DNA, 20 bp, Homo sapiens
  source 1..20: mol_type = other DNA
ctggaaggtggacagcgagg